(19) United States
(12) Patent Application Publication
(10) Pub. No.: US 2009/0247521 A1
(43) Pub. Date: Oct. 1, 2009

(54) SOLUBLE EPOXIDE HYDROLASE INHIBITORS FOR THE TREATMENT OF ENDOTHELIAL DYSFUNCTION

(75) Inventors: Bhasker R. Aavula, Pleasanton, CA (US); Sampath-Kumar Anandan, Oakland, CA (US); Richard D. Gless, JR., Oakland, CA (US); Dinesh Patel, Fremont, CA (US); Gabor Rubanyi, San Diego, CA (US); Yi-Xin Wang, Lafayette, CA (US); Heather K. Webb Hsu, Seattle, WA (US)

Correspondence Address: FOLEY & LARDNER LLP, 975 PAGE MILL ROAD, PALO ALTO, CA, 94304, US

(73) Assignee: Arete Therapeutics, Inc.

(21) Appl. No.: 12/343,358

(22) Filed: Dec. 23, 2008

Related U.S. Application Data
(60) Provisional application No. 61/017,376, filed on Dec. 28, 2007.
(60) Provisional application No. 61/045,216, filed on Apr. 15, 2008.

(51) Int. Cl. A61K 31/445; A61K 31/17; A61K 31/215; A61K 31/19; A61K 31/5377; A61K 31/4545; A61K 31/454; A61K 31/4709; A61P 9/00

(52) U.S. Cl. 514/235.5; 514/588; 514/511; 514/557; 514/329; 514/318; 514/326; 514/314

(57) ABSTRACT

The present invention generally relates to methods useful for a therapy using a class of urea or amide compounds and related compositions, wherein the compound is a soluble epoxide hydrolase inhibitor, for treating and ameliorating the symptoms of diseases related to endothelial dysfunction.

### CROSS-REFERENCE TO RELATED APPLICATIONS

This application claims the benefit under 35 U.S.C. §119(e) of U.S. Provisional Patent Application Nos. 61/017,376, filed on Dec. 28, 2007, and 61/045,216, filed on Apr. 15, 2008, both of which are hereby incorporated by reference in their entirety.

### FIELD OF THE INVENTION

The present invention generally relates to methods useful for treating and ameliorating the symptoms of diseases related to endothelial dysfunction using a therapy of a class of urea or amide compounds and related compositions wherein the compound is a soluble epoxide hydrolase inhibitor (sEH inhibitor). A variety of diseases related to endothelial dysfunction, include, by way of example only, vascular inflammation, atherosclerosis plaque progression/rupture, acute coronary syndrome, coronary-angina, cerebral-subarachnoid hemorrhage, nephropathy, diabetic vasculopathy, autoimmune vasculitis, cerebral vasospasm, transient ischemic attack, peripheral artery occlusive disease, and critical limb ischemia.

### BACKGROUND

The arachidonate cascade is a ubiquitous lipid signaling cascade that liberates arachidonic acid from the plasma membrane lipid reserves in response to a variety of extra-cellular and/or intra-cellular signals. The released arachidonic acid is then available to act as a substrate for a variety of oxidative enzymes that convert it to signaling lipids that have been implicated in inflammation and other diseases. Several commercially available drugs target and disrupt this pathway. Non-steroidal anti-inflammatory drugs (NSAIDS) disrupt the conversion of arachidonic acid to prostaglandins by inhibiting cyclooxygenases (COX1 and COX2). Asthma drugs, such as SINGULAIR™ disrupt the conversion of arachidonic acid to leukotrienes by inhibiting lipoxygenase (LOX).

Certain cytochrome P450-dependent enzymes convert arachidonic acid into a series of epoxide derivatives known as epoxyeicosatrienoic acids (EETs). These EETs are particularly prevalent in endothelium (cells that make up arteries and vascular beds), kidney, and lung. In contrast to many of the end products of the prostaglandin and leukotriene pathways, the EETs are reported to have a variety of anti-inflammatory and anti-hypertensive properties.

While EETs have potent effects in vivo, the epoxide moiety of the EETs is rapidly hydrolyzed into the less active dihydroxyeicosatrienoic acid (DHET) form by an enzyme called soluble epoxide hydrolase (sEH). Inhibition of sEH has been reported to significantly reduce blood pressure in hypertensive animals (see, e.g., Yu et al. Circ. Res. 87:992-8 (2000) and Sinal et al. J. Biol. Chem. 275:40504-10 (2000)), to reduce the production of proinflammatory nitric oxide (NO), cytokines, and lipid mediators, and to contribute to inflammatory resolution by enhancing lipoxin A₄ production in vivo (see Schmelzer et al. Proc. Nat'l Acad. Sci. USA 102 (28):9772-7 (2005)).

One of the consequences of the inflammatory diseases affected by the cascade of cellular and biochemical events related to regulation of sEH activity is endothelial dysfunction which is believed to have an inflammatory component as one of its seminal events. Diseases which are related to endothelial dysfunction include, by way of example, vascular inflammation, atherosclerosis plaque progression/rupture, acute coronary syndrome, coronary-angina, cerebral-subarachnoid hemorrhage, nephropathy, diabetic vasculopathy, autoimmune vasculitis, cerebral vasospasm, transient ischemic attack, peripheral artery occlusive disease, and critical limb ischemia.

This invention provides methods useful for a therapy using a class of urea compounds and related compositions, wherein the compound is a soluble epoxide hydrolase inhibitor, for treating and ameliorating the symptoms of diseases related to endothelial dysfunction.

### SUMMARY OF THE INVENTION

This invention provides methods and compositions useful for a therapy using a class of urea or amide compounds and related compositions, wherein the compound is a soluble epoxide hydrolase inhibitor, for treating and ameliorating the symptoms of diseases related to endothelial dysfunction.

In one aspect, the invention provides methods for treating or ameliorating the diseases related to endothelial dysfunction, such as, but not limited to, vascular inflammation, such as, atherosclerosis plaque progression/rupture and acute coronary syndrome; vasospasm, such as, cerebral vasospasm, coronary-angina and cerebral-subarachnoid hemorrhage; nephropathy, such as, micro-albuminuria; diabetic vasculopathy; autoimmune vasculitis; transient ischemic attack; peripheral artery occlusive disease; and critical limb ischemia, by administering an effective amount of a one or more compounds of Formula I which possess sEH inhibitor activity.

As is apparent to the skilled artisan, the amount and dosing schedule of any therapeutic agent will vary with the disease to be treated, the individual and his or her general health. In one aspect, the invention provides methods for treating a disease or a symptom of a disease related to endothelial dysfunction in a subject, said method comprising administering to a subject in need of such treatment an effective amount of compound of Formula I:

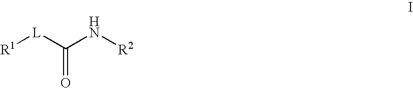

wherein
R¹ and R² independently are selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
L is —NH— or —CR′R″— where R′ and R″ are independently H or alkyl or R′ and R″ together form a C₃-C₆ cycloalkyl ring;
or a stereoisomer, a tautomer, or a pharmaceutically acceptable salt thereof, wherein the compound of Formula I is a soluble epoxide hydrolase inhibitor.

In one aspect are provided methods for treating a disease or a symptom of a disease related to endothelial dysfunction in a subject, said method comprising administering to a subject in need of such treatment an effective amount of a compound of Formula Ia:

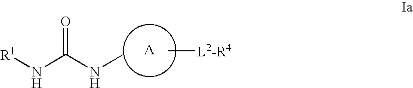

R¹ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
A is cycloalkyl, heterocycloalkyl, aryl, or heteroaryl;
L² is O, C(O), S(O), S(O)₂, or a bond; and
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
or a stereoisomer, tautomer, or pharmaceutically acceptable salt thereof.

In one aspect are provided methods for treating a disease or a symptom of a disease related to endothelial dysfunction in a subject, said method comprising administering to a subject in need of such treatment an effective amount of a compound of Formula II:

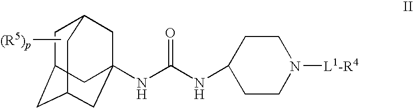

wherein:
L¹ is C(O), S(O), S(O)₂, or a bond;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl, and
R⁵ is hydrogen, halo, or hydroxy; and
p is an integer equal to 0, 1, 2 or 3;
or a stereoisomer, tautomer, or pharmaceutically acceptable salt thereof.

In one embodiment are provided methods for treating a disease or a symptom of a disease related to endothelial dysfunction in a subject, said method comprising administering to a subject in need of such treatment an effective amount of a compound of Formula III:

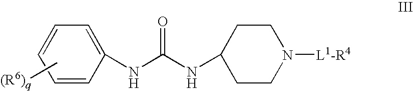

wherein:
L¹ is C(O), S(O), S(O)₂, or a bond;
q is an integer equal to 1, 2, or 3;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl, and
R⁶ is selected from the group consisting of halogen, haloalkyl, alkoxy, and substituted alkoxy;
or a stereoisomer, tautomer, or pharmaceutically acceptable salt thereof.

In one embodiment are provided methods for treating a disease or a symptom of a disease related to endothelial dysfunction in a subject, said method comprising administering to a subject in need of such treatment an effective amount of a compound of Formula IV:

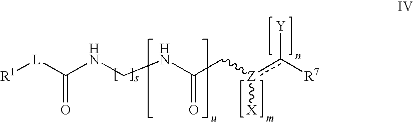

wherein
R¹ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
L is —NH— or —CR′R″— where R′ and R″ are independently hydrogen or alkyl or R′ and R″ together form a C₃-C₆ cycloalkyl ring;
Z is C, O, or NR⁸ where R⁸ is hydrogen or C₁-C₄ alkyl and where when Z is O or NR⁸ then X is absent;
the dotted line is a single or a double bond;
the wavy line is a cis or a trans configuration when the dotted line is a double bond and m and n are 1;
when the dotted line is a single bond and Z is C, then m and n are 2;
s is 0, 1, 2, 3, 4, 5, 6, 7, 8, 9, or 10;
u is 0 or 1;
each of X and Y independently is selected from the group consisting of hydrogen, C₁-C₄ alkyl, substituted C₁-C₄ alkyl, and halo; and
R⁷ is selected from the group consisting of alkyl, substituted alkyl, acyloxy, substituted acyloxy, aminocarbonyl, carboxyl, carboxyl ester, and carboxylic acid isostere,
or a stereoisomer, tautomer, or pharmaceutically acceptable salt thereof.

In one embodiment are provided methods for treating a disease or a symptom of a disease related to endothelial dysfunction in a subject, said method comprising administering to a subject in need of such treatment an effective amount of a compound of Formula V:

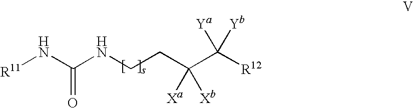

wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —OR¹³, —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo;
or a stereoisomer, tautomer, or pharmaceutically acceptable salt thereof.

In one embodiment, at least one of Y^{a} and Y^{b} is halo or C₁-C₄ alkyl.

In one embodiment are provided methods for treating a disease or a symptom of a disease related to endothelial dysfunction in a subject, said method comprising administering to a subject in need of such treatment an effective amount of a compound of Formula VIa or VIb:

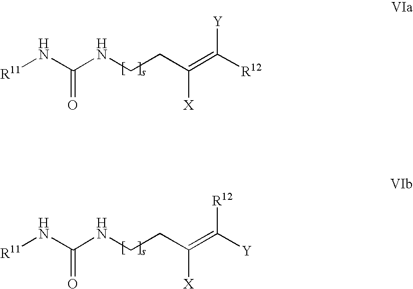

wherein
R¹¹ is selected from the group consisting of cycloalkyl, substituted cycloalkyl, phenyl and substituted phenyl;
is 0, 1, 2, 3, 4, 5, 6, 7, 8, 9, or 10;
R¹² is selected from the group consisting of —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
X and Y are independently selected from the group consisting of hydrogen, C₁-C₄ alkyl, substituted C₁-C₄ alkyl, and halo,
or a stereoisomer, tautomer, or pharmaceutically acceptable salt thereof.

In one embodiment are provided methods for treating a disease or a symptom of a disease related to endothelial dysfunction in a subject, said method comprising administering to a subject in need of such treatment an effective amount of a compound of Formula VII:

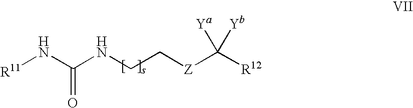

wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —OR¹³, —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
Z is O or NR⁸ where R⁸ is hydrogen or C₁-C₄ alkyl; and Y^{a} and Y^{b} independently are selected from the group consisting of hydrogen, halo, and C₁-C₄ alkyl,
or a stereoisomer, tautomer, or pharmaceutically acceptable salt thereof.

In one embodiment of Formula V, and VII, R¹² is selected from the group consisting of —CH₂OR¹³, —COR¹³, —COOR¹³, —CONR¹³R¹⁴, and carboxylic acid isostere. In the present invention the compounds of Formula I-VII are soluble epoxide hydrolase inhibitors.

In one embodiment, the compound of Formula I-VII is a soluble epoxide hydrolase inhibitor having an IC₅₀ value of less than 25 μM. In one embodiment, the compound of Formula I-VII has an IC₅₀ value of less than 10 μM. In one embodiment, the compound of Formula I-VII has an IC₅₀ value of less than 1 μM.

### DETAILED DESCRIPTION OF THE INVENTION

Throughout this disclosure, various publications, patents and published patent specifications are referenced by an identifying citation. The disclosures of these publications, patents and published patent specifications are hereby incorporated by reference into the present disclosure to more fully describe the state of the art to which this invention pertains.

As used herein, certain terms have the following defined meanings. Terms that are not defined have their art recognized meanings.

As used in the specification and claims, the singular form “a”, “an” and “the” include plural references unless the context clearly dictates otherwise.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

All numerical designations, e.g., pH, temperature, time, concentration, and molecular weight, including ranges, are approximations which are varied (+) or (−) by increments of 0.1. It is to be understood, although not always explicitly stated, that all numerical designations are preceded by the term “about”. It also is to be understood, although not always explicitly stated, that the reagents described herein are merely exemplary and that equivalents of such are known in the art.

“Cis-Epoxyeicosatrienoic acids” (“EETs”) are biomediators synthesized by cytochrome P450 epoxygenases.

“Epoxide hydrolases” (“EH;” EC 3.3.2.3) are enzymes in the alpha/beta hydrolase fold family that add water to 3 membered cyclic ethers termed epoxides.

“Soluble epoxide hydrolase” (“sEH”) is an enzyme which in endothelial, smooth muscle and other cell types converts EETs to dihydroxy derivatives called dihydroxyeicosatrienoic acids (“DHETs”). The cloning and sequence of the murine sEH is set forth in Grant et al., J. Biol. Chem. 268 (23):17628-17633 (1993). The cloning, sequence, and accession numbers of the human sEH sequence are set forth in Beetham et al., Arch. Biochem. Biophys. 305 (1):197-201 (1993). The evolution and nomenclature of the gene is discussed in Beetham et al., DNA Cell Biol. 14 (1):61-71 (1995). Soluble epoxide hydrolase represents a single highly conserved gene product with over 90% homology between rodent and human (Arand et al., FEBS Lett., 338:251-256 (1994)).

“Alkyl” refers to monovalent saturated aliphatic hydrocarbyl groups having from 1 to 10 carbon atoms and alternatively 1 to 6 carbon atoms. In one embodiment, the alkyl is having from 1 to 4 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Alkenyl” refers to straight or branched hydrocarbyl groups having from 2 to 6 carbon atoms and alternatively 2 to 4 carbon atoms and having at least 1 and alternatively from 1 to 2 sites of vinyl (>C═C<) unsaturation. Such groups are exemplified, for example, by vinyl, allyl, and but-3-en-1-yl. Included within this term are the cis and trans isomers or mixtures of these isomers.

“Alkynyl” refers to straight or branched monovalent hydrocarbyl groups having from 2 to 6 carbon atoms and alternatively 2 to 3 carbon atoms and having at least 1 and alternatively from 1 to 2 sites of acetylenic (—C≡C—) unsaturation. Examples of such alkynyl groups include acetylenyl (—C≡CH), and propargyl (—CH₂C≡CH).

“Substituted alkyl” refers to an alkyl group having from 1 to 5, alternatively 1 to 3, or more alternatively 1 to 2 substituents selected from the group consisting of alkenyl, substituted alkenyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Substituted alkenyl” refers to alkenyl groups having from 1 to 3 substituents, and alternatively 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to a vinyl (unsaturated) carbon atom.

“Substituted alkynyl” refers to alkynyl groups having from 1 to 3 substituents, and alternatively 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Acyl includes the “acetyl” group CH₃C(O)—.

“Acylamino” refers to the groups —NRC(O)alkyl, —NRC(O)substituted alkyl, —NRC(O)cycloalkyl, —NRC(O)substituted cycloalkyl, —NRC(O)cycloalkenyl, —NRC(O)substituted cycloalkenyl, —NRC(O)alkenyl, —NRC(O)substituted alkenyl, —NRC(O)alkynyl, —NRC(O)substituted alkynyl, —NRC(O)aryl, —NRC(O)substituted aryl, —NRC(O)heteroaryl, —NRC(O)substituted heteroaryl, —NRC(O)heterocyclic, and —NRC(O)substituted heterocyclic wherein R is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, cycloalkenyl-C(O)O—, substituted cycloalkenyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Substituted amino” refers to the group —NR′R″ where R′ and R″ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, and SO₂-substituted heterocyclic and wherein R′ and R″ are optionally joined, together with the nitrogen bound thereto to form a heterocyclic or substituted heterocyclic group, provided that R′ and R″ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. When R′ is hydrogen and R″ is alkyl, the substituted amino group is sometimes referred to herein as alkylamino. When R′ and R″ are alkyl, the substituted amino group is sometimes referred to herein as dialkylamino. When referring to a monosubstituted amino, it is meant that either R′ or R″ is hydrogen but not both. When referring to a disubstituted amino, it is meant that neither R′ nor R″ are hydrogen.

“Aminocarbonyl” refers to the group —C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonyl” refers to the group —C(S)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NRC(O)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminothiocarbonylamino” refers to the group —NRC(S)NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyloxy” refers to the group —O—C(O)NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyl” refers to the group —SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonyloxy” refers to the group —O—SO₂NR¹⁰R¹¹ where R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminosulfonylamino” refers to the group —NR—SO₂NR¹⁰R¹¹ where R is hydrogen or alkyl and R¹⁰ and R¹¹ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amidino” refers to the group —C(═NR¹²)NR¹⁰R¹¹ where R¹⁰, R¹¹, and R¹² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R¹⁰ and R¹¹ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is at an aromatic carbon atom. Exemplary aryl groups include phenyl and naphthyl.

“Substituted aryl” refers to aryl groups which are substituted with 1 to 5, alternatively 1 to 3, or more alternatively 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Carbonyl” refers to the divalent group —C(O)— which is equivalent to —C(═O)—.

“Carboxy” or “carboxyl” refers to —COOH or salts thereof.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —SO₃H, —SO₂NHR^{k′}, —PO₂(R^{k′})₂, —CN, —PO₃(R^{k′})₂, —OR^{k}, —SR^{k′}, —NHCOR^{k′}, —N(R^{k′})₂, —CONH(O)R^{k′}, —CONHNHSO₂R^{k′}, —COHNSO₂R^{k′}, and —CONR^{k′}CN, where R^{k′} is selected from hydrogen, hydroxyl, halo, haloalkyl, thiocarbonyl, alkoxy, alkenoxy, aryloxy, cyano, nitro, imino, alkylamino, aminoalkyl, thiol, thioalkyl, alkylthio, sulfonyl, alkyl, alkenyl, alkynyl, aryl, aralkyl (-(alkyl)-(aryl)), cycloalkyl, heteroaryl, heterocycle, and CO₂R^{m′} where R^{m′} is hydrogen, alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of carboxylic acid isosteres contemplated by this invention.

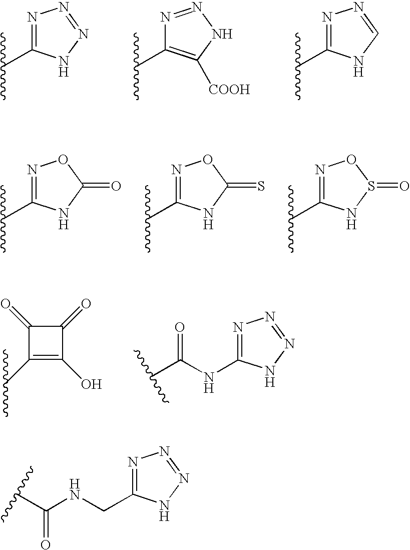

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)amino” refers to the group —NR—C(O)O-alkyl, —NR—C(O)O-substituted alkyl, —NR—C(O)O-alkenyl, —NR—C(O)O-substituted alkenyl, —NR—C(O)O-alkynyl, —NR—C(O)O-substituted alkynyl, —NR—C(O)O-aryl, —NR—C(O)O-substituted aryl, —NR—C(O)O-cycloalkyl, —NR—C(O)O-substituted cycloalkyl, —NR—C(O)O-cycloalkenyl, —NR—C(O)O-substituted cycloalkenyl, —NR—C(O)O-heteroaryl, —NR—C(O)O-substituted heteroaryl, —NR—C(O)O-heterocyclic, and —NR—C(O)O-substituted heterocyclic wherein R is alkyl or hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” refers to the group —CN.

“Cycloalkyl” refers to cyclic alkyl groups of from 3 to 10 carbon atoms having single or multiple cyclic rings including fused, bridged, and spiro ring systems. One or more of the rings can be aryl, heteroaryl, or heterocyclic provided that the point of attachment is through the non-aromatic, non-heterocyclic ring carbocyclic ring. Examples of suitable cycloalkyl groups include, for instance, adamantyl, cyclopropyl, cyclobutyl, cyclopentyl, and cyclooctyl. Other examples of cycloalkyl groups include bicycle[2,2,2,]octanyl, norbornyl, and spiro groups such as spiro[4.5]dec-8-yl:

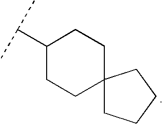

“Cycloalkenyl” refers to non-aromatic cyclic alkyl groups of from 3 to 10 carbon atoms having single or multiple cyclic rings and having at least one >C═C<ring unsaturation and alternatively from 1 to 2 sites of >C═C<ring unsaturation.

“Substituted cycloalkyl” and “substituted cycloalkenyl” refers to a cycloalkyl or cycloalkenyl group having from 1 to 5 or alternatively 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein.

“Cycloalkyloxy” refers to —O-cycloalkyl.

“Substituted cycloalkyloxy” refers to —O-(substituted cycloalkyl).

“Cycloalkylthio” refers to —S-cycloalkyl.

“Substituted cycloalkylthio” refers to —S-(substituted cycloalkyl).

“Cycloalkenyloxy” refers to —O-cycloalkenyl.

“Substituted cycloalkenyloxy refers to —O-(substituted cycloalkenyl).

“Cycloalkenylthio” refers to —S-cycloalkenyl.

“Substituted cycloalkenylthio” refers to —S-(substituted cycloalkenyl).

“Guanidino” refers to the group —NHC(═NH)NH₂.

“Substituted guanidino” refers to —NR¹³C(═NR¹³)N(R¹³)₂ where each R¹³ is independently selected from the group consisting of hydrogen, alkyl, substituted alkyl, aryl, substituted aryl, heteroaryl, substituted heteroaryl, heterocyclic, and substituted heterocyclic and two R¹³ groups attached to a common guanidino nitrogen atom are optionally joined together with the nitrogen bound thereto to form a heterocyclic or substituted heterocyclic group, provided that at least one R¹³ is not hydrogen, and wherein said substituents are as defined herein.

“Halo” or “halogen” refers to fluoro, chloro, bromo and iodo and alternatively is fluoro or chloro.

“Haloalkyl” refers to alkyl groups substituted with 1 to 5, 1 to 3, or 1 to 2 halo groups, wherein alkyl and halo are as defined herein.

“Haloalkoxy” refers to alkoxy groups substituted with 1 to 5, 1 to 3, or 1 to 2 halo groups, wherein alkoxy and halo are as defined herein.

“Haloalkylthio” refers to alkylthio groups substituted with 1 to 5, 1 to 3, or 1 to 2 halo groups, wherein alkylthio and halo are as defined herein.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen and sulfur within the ring. Such heteroaryl groups can have a single ring (e.g., pyridinyl or furyl) or multiple condensed rings (e.g., indolizinyl or benzothienyl) wherein the condensed rings may or may not be aromatic and/or contain a heteroatom provided that the point of attachment is through an atom of the aromatic heteroaryl group. In one embodiment, the nitrogen and/or the sulfur ring atom(s) of the heteroaryl group are optionally oxidized to provide for the N-oxide (N→O), sulfinyl, or sulfonyl moieties. Exemplary heteroaryls include pyridinyl, pyrrolyl, indolyl, thiophenyl, and furanyl.

“Substituted heteroaryl” refers to heteroaryl groups that are substituted with from 1 to 5, alternatively 1 to 3, or more alternatively 1 to 2 substituents selected from the group consisting of the same group of substituents defined for substituted aryl.

“Heteroaryloxy” refers to —O-heteroaryl.

“Substituted heteroaryloxy” refers to the group —O-(substituted heteroaryl).

“Heteroarylthio” refers to the group —S-heteroaryl.

“Substituted heteroarylthio” refers to the group —S-(substituted heteroaryl).

“Heterocycle” or “heterocyclic” or “heterocycloalkyl” or “heterocyclyl” refers to a saturated or partially saturated, but not aromatic, group having from 1 to 10 ring carbon atoms and from 1 to 4 ring heteroatoms selected from the group consisting of nitrogen, sulfur, or oxygen. Heterocycle encompasses single ring or multiple condensed rings, including fused bridged and spiro ring systems. In fused ring systems, one or more the rings can be cycloalkyl, aryl, or heteroaryl provided that the point of attachment is through the non-aromatic ring. In one embodiment, the nitrogen and/or sulfur atom(s) of the heterocyclic group are optionally oxidized to provide for the N-oxide, sulfinyl, or sulfonyl moieties.

“Substituted heterocyclic” or “substituted heterocycloalkyl” or “substituted heterocyclyl” refers to heterocyclyl groups that are substituted with from 1 to 5 or alternatively 1 to 3 of the same substituents as defined for substituted cycloalkyl.

“Heterocyclyloxy” refers to the group —O-heterocyclyl.

“Substituted heterocyclyloxy” refers to the group —O-(substituted heterocyclyl).

“Heterocyclylthio” refers to the group —S-heterocyclyl.

“Substituted heterocyclylthio” refers to the group —S-(substituted heterocyclyl).

Examples of heterocycle and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O) or (—O⁻).

“Spiro ring systems” refers to bicyclic ring systems that have a single ring carbon atom common to both rings.

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-cycloalkenyl, —SO₂-substituted cylcoalkenyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Substituted sulfonyl includes groups such as methyl-SO₂—, phenyl-SO₂—, and 4-methylphenyl-SO₂—. The term “alkylsulfonyl” refers to —SO₂-alkyl. The term “haloalkylsulfonyl” refers to —SO₂-haloalkyl where haloalkyl is defined herein. The term “(substituted sulfonyl)amino” refers to —NH(substituted sulfonyl) wherein substituted sulfonyl is as defined herein.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-cycloalkenyl, —OSO₂-substituted cylcoalkenyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, cycloalkenyl-C(S)—, substituted cycloalkenyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Thiol” refers to the group —SH.

“Thiocarbonyl” refers to the divalent group —C(S)— which is equivalent to —C(═S)—.

“Thione” refers to the atom (═S).

“Alkylthio” refers to the group —S-alkyl wherein alkyl is as defined herein.

“Substituted alkylthio” refers to the group —S-(substituted alkyl) wherein substituted alkyl is as defined herein.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

“Stereoisomer” or “stereoisomers” refer to compounds that differ in the chirality at one or more stereocenters. Stereoisomers include enantiomers and diastereomers.

“Tautomer” refer to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Prodrug” refers to any derivative of a compound of the embodiments that is capable of directly or indirectly providing a compound of the embodiments or an active metabolite or residue thereof when administered to a subject. Particularly favored derivatives and prodrugs are those that increase the bioavailability of the compounds of the embodiments when such compounds are administered to a subject (e.g., by allowing an orally administered compound to be more readily absorbed into the blood) or which enhance delivery of the parent compound to a biological compartment (e.g., the brain or lymphatic system) relative to the parent species. Prodrugs include ester forms of the compounds of the invention. Examples of ester prodrugs include formate, acetate, propionate, butyrate, acrylate, and ethylsuccinate derivatives. A general overview of prodrugs is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, and tetraalkylammonium; and when the molecule contains a basic functionality, salts of organic or inorganic acids, such as hydrochloride, hydrobromide, tartrate, mesylate, acetate, maleate, and oxalate.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically-acceptable carrier” encompasses any of the standard pharmaceutical carriers, such as a phosphate-buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI., 15th Ed. (Mack Publ. Co., Easton (1975)).

An “excipient” refers to an inert substance added to a pharmaceutical composition to further facilitate administration of the active ingredient.

A “subject,” “individual” or “patient” is used interchangeably herein, and refers to a vertebrate, for example a mammal or alternatively a human. Mammals include, but are not limited to, murines, rats, simians, humans, farm animals, sport animals and pets.

An “effective amount” is used synonymously with a “therapeutically effective amount” and intends an amount sufficient to effect beneficial or desired results. An effective amount can be administered in one or more administrations, applications, or dosages. It also refers to an amount that will elicit one or more of the following effects: reducing autoimmune-induced inflammation as indicated by reduction in redness, fever, edema, swelling and pain systemically and/or locally, decreasing the levels of inflammatory cytokines and increasing the levels of anti-inflammatory cytokines.

An “endothelial dysfunction” as used herein refers to reduced endothelium-dependent vasodilation. The diseases related to endothelial dysfunction, include, by way of example only, vascular inflammation, atherosclerosis plaque progression/rupture, acute coronary syndrome, coronary-angina, cerebral-subarachnoid hemorrhage, nephropathy, diabetic vasculopathy, autoimmune vasculitis, cerebral vasospasm, transient ischemic attack, peripheral artery occlusive disease, and critical limb ischemia.

“Vascular inflammation” as used herein refers to a low-grade inflammation in the blood vessels caused by a variety of factors, including but are not limited to, immune complexes (autoimmune), viruses, bacteria, hormones (Ang II), toxins (LPS) etc.

“Autoimmune Disease” refers to conditions where inflammation and other conditions associated with malfunction result at least in part by faulty recognition of self by the immune system. Examples of autoimmune diseases include, for example, autoimmune vasculitis, rheumatoid arthritis, celiac disease, Crohn's disease, inflammatory bowel disease, pancreatitis, systemic lupus erythematosus, Sjogren's syndrome, myocarditis, Hashimoto's thyroiditis and multiple sclerosis. The autoimmune vasculitis include but are not limited to, Scleroderma, Lupus, Behcet syndrome, Takayashu arteritis, Churg-Strauss Syndrome, Cutaneous vasculitis, thrombangitis obliterans (Reynauds syndrome), sickle cell anemia, and beta thalasemia.

“Treating” or “treatment” of a disease or condition will depend on the disease or condition to be treated and the individual to be treated. In general, treatment intends one or more of (1) inhibiting the progression of the disease or condition as measured by clinical or sub-clinical parameters, (2) arresting the development of the disease as measured by clinical or sub-clinical parameters, (3) ameliorating or causing regression of the disease or condition as measured by clinical or sub-clinical parameters, or (4) reducing pain or discomfort for the subject as measured by clinical parameters.

As used herein, “a subject in need thereof” intends a subject such as a human patient that presents characteristic symptoms of diseases related to endothelial dysfunction or alternatively has been diagnosed by a health care professional as suffering from such diseases.

As used herein, the “dotted line” means:

[symbol]

As used herein, the “wavy line” means:

[symbol]

### 1. Therapeutic Methods

This invention provides methods and compositions that treat, reduce or ameliorate the diseases or the symptoms of diseases related to endothelial dysfunction using one or more compound(s) of Formula I-VII which possess sEH inhibitor activity.

The endothelium is a cellular layer lining the walls of blood vessels of a mammal. It is a highly specialized interface between blood and underlying tissues and has a number of functions, including: control of haemostasis by inhibiting platelet aggregation (antithrombotic and regulating the coagulation and fibrolinolytic systems); control of vascular tone, and hence blood flow; control of blood vessel smooth muscle growth; and selective permeability to cells and proteins.

Normally, the endothelium maintains vascular homeostasis by responding to physiological stimuli, for example, changes in blood flow, oxygen tension etc., by adaptive alteration of function. Dysfunctional endothelium has an impaired response to such physiological stimuli, and can ultimately lead to medical disorders. A number of subsets of endothelial dysfunction have been recognized, including Endothelial Activation, and Endothelial-mediated Vasodilatory Dysfunction (see De Caterina “Endothelial dysfunctions: common denominators in vascular disease”. Current Opinions in Lipidology 11:9-23, (2000)).

Endothelial activation may lead to the initiation of atherosclerosis and is a process whereby there is an inappropriate up-regulation and expression of cell attraction and cell adhesion molecules on endothelial cells. This particularly involves the Macrophage Chemoattractant Protein-1 (MCP-1), chemoattractants for lymphocytes (IP-10, MIG, I-TAG), the Vascular Cell Adhesion Molecule-1 (VCAM-1), IL-1, IL-6, TNFα, and ICAM-1, to which the monocytes and lymphocytes adhere. Once adherent, the leucocytes enter the artery wall. The monocytes and lymphocytes are recruited to the intima (sub-endothelial layers) of the blood vessels by these cell attraction and cell adhesion molecules of the activated endothelium during the early stages of atherosclerosis (see Libby, P. “Changing concepts of atherogenesis,” Journal of Internal Medicine 247:349-358, (2000))

Endothelial-mediated Vasodilatory Dysfunction is characterized by a reduction or loss of endothelium-dependent vasodilation and involves “decreased nitric oxide bioavailability” (decreased production, increased destruction and/or decreased sensitivity to nitric oxide). (De Caterina (2000), cited above). Nitric oxide induces vasodilation by relaxing the smooth muscle cells of the blood vessel wall. Endothelial-mediated Vasodilatory Dysfunction can be measured as a reduction in vasodilation in response to acetylcholine, or as a reduced vasodilatory response following occlusion of arterial blood flow (reactive hyperaemia) for example using a sphygmomanometer cuff. As well as leading to a reduction in vasodilation, decreased endothelial nitric oxide bioavailability can also result in an increase in the production of vaso-constriction and hypertension. Platelet aggregation is inhibited by nitric oxide, hence a decrease in nitric oxide bioavailability can lead to an increase in platelet aggregation and consequent thrombosis. These are just a few examples of how decreased nitric oxide bioavailability resulting from Endothelial-mediated Vasodilatory Dysfunction can have pathological consequences.

A variety of diseases related to endothelial dysfunction that can be treated in the present invention, include, by way of example only, vascular inflammation, such as, atherosclerosis plaque progression/rupture and acute coronary syndrome; vasospasm, such as, coronary-angina and cerebral-subarachnoid hemorrhage; nephropathy, such as, micro-albuminuria; diabetic vasculopathy; and autoimmune vasculitis. In one embodiment, the autoimmune vasculitis relates to scleroderma, lupus, behcet syndrome, takayashu arteritis, churg-strauss syndrome, cutaneous vasculitis, and thrombangitis obliterans (Reynaud's syndrome). In one embodiment, autoimmune vasculitis is associated with sickle cell anemia and beta thalasemia.

Sickle cell anemia is characterized by several aspects that make it a disease that may be positively impacted by inhibition of sEH. Although the anemia is congenital, the acute sickling events lead to the actual issues with the disease including vascular inflammation, stroke and renal damage. Vascular inflammation may be considered a key characteristic of this disease. Stroke is a co-morbidity in sickle cell anemia that has potential to be positively impacted by sEH inhibitors. Additionally, it is also characterized by leading to a wide range of glomerular and tubulointerstitial nephropathies. Finally, an sEH inhibitor can be anti-thrombotic which can positively impact the primary mortality.

In one embodiment, the invention provides methods and compositions that treat, reduce or ameliorate the diseases or the symptoms of diseases related to vascular inflammation, using one or more compound(s) of Formula I-VII.

Functional tests/diagnosis normally used to screen for diseases related to endothelial dysfunction include but are not limited to, flow-mediated arterial dilation (FMAD) usually measured non-invasively in the patients's forearm (brachial artery) and measurement of acetylcholine-induced arterial dilation. The biochemical markers measured in patients blood/plasma include but are not limited to, soluble Vascular Cell Adhesion Molecule-1 (VCAM-1), Intercellular Adhesion Molecule-1 (ICAM-1), Platelet/endothelial Cell Adhesion Molecule-1 (PECAM-1) and von Willebrand Factor (vWF). Functional tests/diagnosis normally used to screen for diseases related to vascular inflammation include, but are not limited to, blood/plasma markers such as above and/or TNFα, IL-1, IL-6, MCP-1, NOx, etc. and clinical symptoms.

The amount, dosing schedule and route of administration of composition can be determined by the treating physician and will vary with the active agent, and its pharmacological properties, condition to be treated, the severity of the condition, the overall general health of the subject, age, weight and sex of the subject. It should be understood that an effective amount to achieve the desired response is administered.

One can determine if the treatment has been effective for its defined purpose by noting one or more clinical symptoms such as a reduction in pain, redness, swelling and loss of mobility or function. Administration of compositions of the invention can be further selected on their ability to reduce clinical symptoms by at least 50%, or alternatively, at least by about 60% or alternatively by at least about 70%, or alternatively by at least about 75%, or alternatively by at least about 80%, or alternatively by at least about 85%, or alternatively by at least about 90%, or alternatively by at least about 95%, of pre-administration levels in the subject.

Also provided is a medicament comprising one or more compound(s) of Formula I-VII for use in treating a disease or disorder as described above, which can be identified by noting any one or more clinical or sub-clinical parameters.

### 2. Compounds for Use in the Therapeutic Methods

In each of the above method embodiments, an effective amount of a composition containing one or more compound(s) of Formula I-VII is administered to a subject in need thereof. The compounds are described by at least one of the following general or specific formula.

In one aspect, the compound is a member of the group of Formula I:

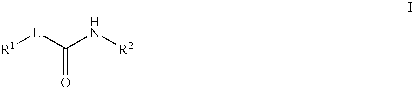

wherein
R¹ and R² independently are selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
L is —NH— or —CR′R″— where R′ and R″ are independently H or alkyl or R′ and R″ together form a C₃-C₆ cycloalkyl ring;
or a stereoisomer, a tautomer, or a pharmaceutically acceptable salt thereof, wherein the compound of Formula I is a soluble epoxide hydrolase inhibitor.

In one embodiment, R¹ is adamantyl or substituted adamantyl. In one embodiment, R¹ is adamantyl.

In one embodiment, L is —NH—. In one embodiment, L is —CR′R″— where R′ and R″ are independently H or alkyl or R′ and R″ together form a C₃-C₆ cycloalkyl ring. In some embodiments, L is CH₂.

In one embodiment, R¹ is phenyl or substituted phenyl. In one embodiment, R¹ is phenyl. In another embodiment, R¹ is substituted phenyl.

In one embodiment, R² is substituted heterocycloalkyl. In one embodiment, heterocycloalkyl is containing one or more nitrogen as a hetero atom. In still another embodiment, R² is

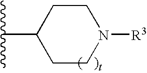

wherein
R³ is L¹-R⁴ where L¹ is C(O), S(O), S(O)₂, or a bond and R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
t is an integer equal to 0, 1 or 2.

In one embodiment, L¹ is C(O). In another embodiment, L¹ is S(O). In another embodiment, L¹ is S(O)₂. In yet another embodiment, L¹ is a bond.

In one embodiment, R⁴ is C₁-C₃ alkyl, phenyl, or substituted phenyl.

In one aspect are provided methods for treating a disease or a symptom of a disease related to endothelial dysfunction in a subject, said method comprising administering to a subject in need of such treatment an effective amount of a compound of Formula Ia:

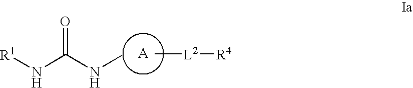

R¹ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
A is cycloalkyl, heterocycloalkyl, aryl, or heteroaryl;
L² is O, C(O), S(O), S(O)₂, or a bond; and
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
or a stereoisomer, tautomer, or pharmaceutically acceptable salt thereof.

In some embodiments, A is cyclohexyl, piperidinyl, phenyl, or pyridinyl. In some embodiments, A is cyclohexyl. In some embodiments, A is piperidinyl. In some embodiments, A is phenyl. In some embodiments, A is pyridinyl.

In some embodiments, L² is O. In some embodiments, L² is C(O). In some embodiments, L² is S(O). In some embodiments, L² is S(O)₂. In some embodiments, L² is a bond.

In one aspect, the compound is of Formula II:

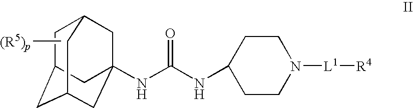

wherein:
L¹ is C(O), S(O), S(O)₂, or a bond;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl, and
R⁵ is hydrogen, halo, or hydroxy; and
p is an integer equal to 0, 1, 2 or 3;
or a stereoisomer, tautomer, or pharmaceutically acceptable salt thereof.

In one embodiment, L¹ is C(O). In another embodiment, L¹ is S(O). In another embodiment, L¹ is S(O)₂. In another embodiment, L¹ is a bond.

In one embodiment, p is 1, 2, or 3.

In one embodiment, R⁴ is C₁-C₃ alkyl, phenyl, substituted phenyl, or heteroaryl. In one embodiment, R⁵ is hydrogen or fluoro. In one embodiment, R⁴ is C₁-C₃ alkyl, phenyl, or substituted phenyl and R⁵ is hydrogen or fluoro.

In one aspect, the compound is of Formula III:

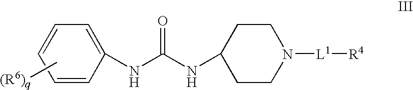

wherein:
L¹ is C(O), S(O), S(O)₂, or a bond;
q is an integer equal to 1, 2, or 3;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl, and
R⁶ is selected from the group consisting of halogen, haloalkyl, alkoxy, and substituted alkoxy;
or a stereoisomer, tautomer, or pharmaceutically acceptable salt thereof.

In one embodiment, L¹ is C(O). In another embodiment, L¹ is S(O). In another embodiment, L¹ is S(O)₂. In another embodiment, L¹ is a bond.

In one embodiment, q is 1. In one embodiment, q is 2.

In another embodiment, R⁴ is C₁-C₃ alkyl, substituted C₁-C₃ alkyl, phenyl, substituted phenyl, heteroaryl, or substituted heteroaryl.

In another embodiment, R⁶ is halogen, CF₃, or OCF₃.

In one aspect, the compound is of Formula IV:

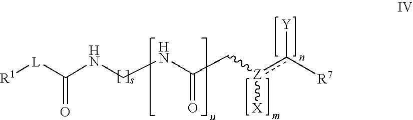

wherein
R¹ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
L is —NH— or —CR′R″— where R′ and R″ are independently hydrogen or alkyl or R′ and R″ together form a C₃-C₆ cycloalkyl ring;
Z is C, O, or NR⁸ where R⁸ is hydrogen or C₁-C₄ alkyl and where when Z is O or NR⁸ then X is absent;
the dotted line is a single or a double bond;
the wavy line is a cis or a trans configuration when the dotted line is a double bond and m and n are 1;
when the dotted line is a single bond and Z is C, then m and n are 2;
s is 0, 1, 2, 3, 4, 5, 6, 7, 8, 9, or 10;
u is 0 or 1;
each of X and Y independently is selected from the group consisting of hydrogen, C₁-C₄ alkyl, substituted C₁-C₄ alkyl, and halo; and
R⁷ is selected from the group consisting of alkyl, substituted alkyl, acyloxy, substituted acyloxy, aminocarbonyl, carboxyl, carboxyl ester, and carboxylic acid isostere,
or a stereoisomer, tautomer, or pharmaceutically acceptable salt thereof.

In one embodiment, when the dotted line [symbol] is the single bond, Z is C, and u is 0, then at least one of Y is halo or C₁-C₄ alkyl.

In one embodiment, when Z is C and u is 1, then each of X and Y independently is hydrogen or C₁-C₄ alkyl.

In one embodiment, R¹ is cycloalkyl, or substituted cycloalkyl. In one embodiment, the substituted cycloalkyl, is substituted with 1 to 3 substituents independently selected from the group consisting of halo and alkyl. In one embodiment, the substituted cycloalkyl, is substituted with 1 to 3 substituents independently selected from the group consisting of fluoro and methyl.

In one embodiment, R¹ is selected from the group consisting of cyclohexyl, substituted cyclohexyl, cyclooctyl, spiro[4.5]decan-8-yl, and 4-methylbicyclo[2.2.2]octan-1-yl.

In one embodiment, R¹ is adamantyl or substituted adamantyl. In one embodiment, R¹ is phenyl or substituted phenyl.

In one embodiment, R¹ is phenyl substituted with 1 to 5 substituents independently selected from the group consisting of hydrogen, halo, alkyl, acyl, acyloxy, carboxyl ester, acylamino, aminocarbonyl, aminocarbonylamino, aminocarbonyloxy, aminosulfonylamino, (carboxyl ester)amino, aminosulfonyl, (substituted sulfonyl)amino, haloalkyl, haloalkoxy, haloalkylthio, cyano, and alkylsulfonyl.

In one embodiment, R¹ is phenyl substituted with 1 to 5 substituents independently selected from the group consisting of fluoro, trifluomethyl, and trifluoromethoxy.

In one embodiment, R¹ is selected from the group consisting of phenyl, 3-trifluoromethylphenyl, 4-trifluoromethylphenyl, 3-trifluoromethoxyphenyl, 4-trifluoromethoxyphenyl, 4-fluorophenyl, 4-chlorophenyl, 4-bromophenyl, 3-fluorophenyl, 3-chlorophenyl and 3-bromophenyl.

In one embodiment, L is —NH—. In one embodiment, L is —CR′R″— where R′ and R″ are independently H or alkyl or R′ and R″ together form a C₃-C₆ cycloalkyl ring.

In one embodiment, s is 2, 3, 4, 5, 6, 7, or 8. In one embodiment, s is 4.

In one embodiment, u is 0.

In one embodiment, u is 1.

In one embodiment, when the dotted line [symbol] is the single bond, Z is C, and u is 0, then at least one of Y is halo or C₁-C₄ alkyl. In one embodiment, when Z is C and u is 1, then each of X and Y independently is hydrogen or C₁-C₄ alkyl.

In a embodiment, when Z is C and u is 0, then at least one of Y is methyl. In a embodiment, when Z is C and u is 0, then at least one of Y is fluoro.

In one embodiment, R⁷ is substituted alkyl. In one embodiment, substituted alkyl is —CH₂OR⁹ where R⁹ is hydrogen or C₁-C₄ alkyl.

In one embodiment, R⁷ is —COOR¹⁰ where R¹⁰ is hydrogen, or C₁-C₄ alkyl.

In one embodiment, R⁷ is —CONH₂.

In one embodiment, R⁷ is —COR⁹ where R⁹ is hydrogen, or C₁-C₄ alkyl.

In one aspect, the compound is of Formula V:

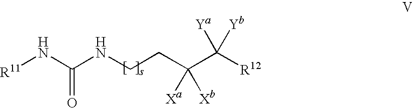

wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —OR¹³, —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo;
or a stereoisomer, tautomer, or pharmaceutically acceptable salt thereof.

In one embodiment, at least one of Y^{a} and Y^{b} is halo or C₁-C₄ alkyl. In one embodiment, R¹¹ is cycloalkyl or substituted cycloalkyl.

In one embodiment, R¹¹ is selected from the group consisting of cyclohexyl, substituted cyclohexyl, cyclooctyl, spiro[4.5]decan-8-yl, and 4-methylbicyclo[2.2.2]octan-1-yl.

In one embodiment, R¹¹ is adamantyl or substituted adamantyl. In one embodiment, R¹¹ is phenyl or substituted phenyl.

In one embodiment, R¹¹ is selected from the group consisting of phenyl, 3-trifluoromethylphenyl, 4-trifluoromethylphenyl, 3-trifluoromethoxyphenyl, 4-trifluoromethoxyphenyl, 4-fluorophenyl, 4-chlorophenyl, 4-bromophenyl, 3-fluorophenyl, 3-chlorophenyl and 3-bromophenyl.

In one embodiment, R¹¹ is phenyl substituted with 1 to 5 substituents independently selected from the group consisting of fluoro, trifluomethyl, and trifluoromethoxy.

In one embodiment, X^{a}, X^{b}, and Y^{a} are hydrogen and Y^{b} is halo. In one embodiment, Y^{b} is fluoro.

In one embodiment, X^{a} and X^{b} are hydrogen, Y^{a} is halo and Y^{b} is halo. In one embodiment, Y^{a} and Y^{b} are fluoro.

In one embodiment, X^{a}, X^{b}, and Y^{a} are hydrogen and Y^{b} is alkyl. In one embodiment, Y^{b} is methyl.

In one embodiment, X^{a} and X^{b} are hydrogen and Y^{a} and Y^{b} are alkyl. In one embodiment, Y^{a} and Y^{b} are methyl.

In one embodiment, Y^{a}, Y^{b}, and X^{a} are hydrogen and X^{b} is alkyl. In one embodiment, Y^{a}, Y^{b}, and X^{a} are hydrogen and X^{b} is methyl.

In one embodiment, Y^{a} and Y^{b} are hydrogen and X^{a} and X^{b} are alkyl. In one embodiment, X^{a} and X^{b} are methyl.

In one embodiment, s is 2, 3, 4, 5, 6, 7, or 8. In one embodiment, s is 4.

In one embodiment, R¹² is —CH₂OR¹³ where R¹³ is selected from the group consisting of hydrogen and methyl.

In one embodiment, R¹² is —COOR¹³ where R¹³ is selected from the group consisting of hydrogen, methyl, ethyl, i-propyl, tert-butyl, 2,2,2-trimethylethyl, and dimethylaminoethyl.

In one embodiment, R¹² is —COR¹³ where R¹³ is selected from the group consisting of hydrogen and methyl.

In one embodiment, R¹² is —CONH₂.

In one aspect, the compound is of Formula VIa or VIb:

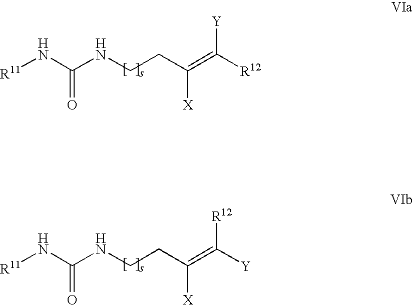

wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
X and Y independently are selected from the group consisting of hydrogen, C₁-C₄ alkyl, substituted C₁-C₄ alkyl, and halo,
or a stereoisomer, tautomer, or pharmaceutically acceptable salt thereof.

In one embodiment, R¹¹ is cycloalkyl or substituted cycloalkyl.

In one embodiment, R¹¹ is selected from the group consisting of cyclohexyl, substituted cyclohexyl, cyclooctyl, spiro[4.5]decan-8-yl, and 4-methylbicyclo[2.2.2]octan-1-yl.

In one embodiment, R¹¹ is adamantyl or substituted adamantyl. In one embodiment, R¹¹ is phenyl or substituted phenyl.

In one embodiment, R¹¹ is selected from the group consisting of phenyl, 4-fluorophenyl, 4-chlorophenyl, 4-bromophenyl, 3-fluorophenyl, 3-chlorophenyl, 3-bromophenyl, 3-trifluoromethylphenyl, 4-trifluoromethylphenyl, 3-trifluoromethoxyphenyl, and 4-trifluoromethoxyphenyl.

In one embodiment, R¹¹ is phenyl substituted with 1 to 5 substituents independently selected from the group consisting of fluoro, trifluomethyl, and trifluoromethoxy.

In one embodiment, s is 3, 4, or 5. In one embodiment, s is 4.

In one embodiment, X is hydrogen. In one embodiment, Y is hydrogen, fluoro, or methyl.

In one embodiment, R¹² is —CH₂OR¹³ where R¹³ is selected from the group consisting of hydrogen and methyl.

In one embodiment, R¹² is —COOR¹³ where R¹³ is selected from the group consisting of hydrogen, methyl, ethyl, i-propyl, tert-butyl, 2,2,2-trimethylethyl, and dimethylaminoethyl.

In one embodiment, R¹² is —COR¹³ where R¹³ is selected from the group consisting of hydrogen and methyl.

In one embodiment, R¹² is —CONH₂.

In one aspect, the compound is of Formula VII:

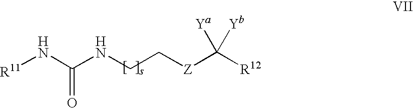

wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
Z is O or NR⁸ where R⁸ is hydrogen or C₁-C₄ alkyl; and Y^{a} and Y^{b} independently are selected from the group consisting of hydrogen, halo, and C₁-C₄ alkyl,
or a stereoisomer, tautomer, or pharmaceutically acceptable salt thereof.

In one embodiment of Formula V, R¹² is selected from the group consisting of —CH₂OR¹³, COR¹³, —COOR¹³, —CONR¹³R¹⁴, and carboxylic acid isostere.

In one embodiment, R¹¹ is cycloalkyl or substituted cycloalkyl.

In one embodiment, R¹¹ is adamantyl or substituted adamantyl.

In one embodiment, R¹¹ is phenyl or substituted phenyl.

In one embodiment, R¹¹ is phenyl substituted with 1 to 5 substituents independently selected from the group consisting of fluoro, trifluomethyl, and trifluoromethoxy.

In one embodiment, R¹¹ is selected from the group consisting of phenyl, 4-fluorophenyl, 4-chlorophenyl, 4-bromophenyl, 3-fluorophenyl, 3-chlorophenyl, 3-bromophenyl, 3-trifluoromethylphenyl, 4-trifluoromethylphenyl, 3-trifluoromethoxyphenyl, and 4-trifluoromethoxyphenyl.

In one embodiment, s is 4, 6, or 8.

In one embodiment, s is 4.

In one embodiment, Z is O.

In one embodiment, Z is NCH₃.

In one embodiment, Y^{a} and Y^{b} independently are fluoro.

In one embodiment, Y^{a} and Y^{b} independently are hydrogen or methyl. In one embodiment, both Y^{a} and Y^{b} are hydrogen,

In one embodiment, R¹² is —CH₂OR¹³ where R¹³ is selected from the group consisting of hydrogen or methyl.

In one embodiment, R¹² is —COOR¹³ where R¹³ is selected from the group consisting of hydrogen, methyl, ethyl, i-propyl, tert-butyl, 2,2,2-trimethylethyl, and dimethylaminoethyl.

In one embodiment, R¹² is —CONH₂.

In one embodiment, R¹² is —COR¹³ where R¹³ is selected from the group consisting of hydrogen and methyl.

In one embodiment, the invention provides a prodrug of the compounds of Formula I, II, III, IV, V, VI, or VII.

In one embodiment, the compounds used in the methods of the present invention are selected from
(Z)-1-(7-fluoro-8-hydroxyoct-6-enyl)-3-(adamantyl)urea;
(Z)-methyl 2-fluoro-8-(3-adamantylureido)oct-2-enoate;
(Z)-ethyl 2-fluoro-8-(3-adamantylureido)oct-2-enoate;
(Z)-isopropyl 2-fluoro-8-(3-adamantylureido)oct-2-enoate;
(Z)-2-fluoro-8-(3-adamantylureido)oct-2-enoic acid;
(Z)-2-fluoro-8-(3-adamantylureido)oct-2-enamide;
(Z)-1-(7-fluoro-8-methoxyoct-6-enyl)-3-adamantylurea;
(Z)-t-butyl 2-fluoro-8-(3-adamantylureido)oct-2-enoate;
(Z)-1-(7-fluoro-8-hydroxyoct-6-enyl)-3-(4-(trifluoromethyl)phenyl)urea;
(Z)-1-(7-fluoro-8-hydroxyoct-6-enyl)-3-(4-(trifluoromethoxy)phenyl)urea;
(Z)-1-(7-fluoro-8-hydroxyoct-6-enyl)-3-(4-fluorophenyl)urea;
(Z)-2-fluoro-8-(3-(4-fluorophenyl)ureido)oct-2-enamide;
(Z)-ethyl 2-fluoro-8-(3-(4-fluorophenyl)ureido)oct-2-enoate;
(Z)-2-fluoro-8-(3-(4-fluorophenyl)ureido)oct-2-enoic acid;
(Z)-2-fluoro-8-(3-(4-(trifluoromethoxy)phenyl)ureido)oct-2-enoic acid;
(Z)-2-fluoro-8-(3-(4-(trifluoromethoxy)phenyl)ureido)oct-2-enamide;
(Z)-ethyl 2-fluoro-8-(3-(4-(trifluoromethoxy)phenyl)ureido)oct-2-enoate;
(Z)-ethyl 2-fluoro-8-(3-(4-(trifluoromethyl)phenyl)ureido)oct-2-enoate;
2-fluoro-6-(3-adamantylureido)hexanoic acid;
Ethyl 2-fluoro-6-(3-adamantylureido)hexanoate;
1-(7-fluoro-8-hydroxyoctyl)-3-(adamantyl)urea;
1-(7,7-difluoro-8-hydroxyoctyl)-3-(adamantyl)urea;
ethyl 2,2-difluoro-8-(3-adamantylureido)octanoate;
methyl 2-fluoro-8-(3-adamantylureido)octanoate;
ethyl 2-fluoro-8-(3-adamantylureido)octanoate;
isopropyl 2-fluoro-8-(3-adamantylureido)octanoate;
2-fluoro-8-(3-adamantylureido)octanoic acid;
t-butyl 2-fluoro-8-(3-adamantylureido)octanoate;
2-fluoro-8-(3-adamantylureido)octanamide;
1-(7-fluoro-8-methoxyoctane)-3-adamantylurea;
1-(7-fluoro-8-oxononyl)-3-adamantylurea;
2-fluoro-12-(3-adamantylureido)dodecanoic acid;
Ethyl 2-fluoro-12-(3-adamantylureido)dodecanoate;
2-fluoro-10-(3-adamantylureido)decanoic acid;
Ethyl 2-fluoro-10-(3-adamantylureido)decanoate;
ethyl 2-fluoro-8-(3-(4-fluorophenyl)ureido)octanoate;
2-fluoro-8-(3-(4-fluorophenyl)ureido)octanoic acid;
2-fluoro-8-(3-(4-fluorophenyl)ureido)octanamide;
2-fluoro-8-(3-(4-(trifluoromethoxy)phenyl)ureido)octanoic acid;
ethyl 2-fluoro-8-(3-(4-(trifluoromethoxy)phenyl)ureido)octanoate;
ethyl 2-fluoro-8-(3-(4-(trifluoromethyl)phenyl)ureido)octanoate;
ethyl 2-fluoro-8-(3-(4-fluorophenyl)ureido)octanoate;
8-(3-(4,4-dimethylcyclohexyl)ureido)-2-fluorooctanoic acid;
ethyl 8-(3-(4,4-dimethylcyclohexyl)ureido)-2-fluorooctanoate;
8-(3-cyclooctylureido)-2-fluorooctanoic acid;
ethyl 8-(3-cyclooctylureido)-2-fluorooctanoate;
8-(3-(4,4-difluorocyclohexyl)ureido)-2-fluorooctanoic acid;
ethyl 8-(3-(4,4-difluorocyclohexyl)ureido)-2-fluorooctanoate;
2-fluoro-8-(3-spiro[4.5]decan-8-ylureido)octanoic acid;
ethyl 2-fluoro-8-(3-spiro[4.5]decan-8-ylureido)octanoate;
2-fluoro-8-(3-(4-methylbicyclo[2.2.2]octan-1-yl)ureido)octanoic acid;
ethyl 2-fluoro-8-(3-(4-methylbicyclo[2.2.2]octan-1-yl)ureido)octanoate;
methyl 2,2-dimethyl-11-(3-(4-(trifluoromethyl)phenyl)ureido)undecanoate;
2,2-dimethyl-11-(3-(4-(trifluoromethyl)phenyl)ureido)undecanoic acid;
1-(8-hydroxy-8-methylnonyl)-3-adamantylurea;
1-(8-hydroxy-9,9-dimethyldecyl)-3-adamantylurea;
(E)-ethyl 8-(3-adamantylureido)oct-2-enoate;
ethyl 2-methyl-8-(3-adamantylureido)octanoate;
1-(5-(2-hydroxyethoxy)pentyl)-3-adamantylurea;
methyl 2-(methyl(9-(3-(4-(trifluoromethyl)phenyl)ureido)nonyl)amino)acetate;
methyl 2-(methyl(9-(3-adamantylureido)nonyl)amino)acetate;
2-(methyl(9-(3-(4-(trifluoromethyl)phenyl)ureido)nonyl)amino)acetamide;
2-(methyl(9-(3-adamantylureido)nonyl)amino)acetamide;
ethyl 2,2-difluoro-2-(5-(3-adamantylureido)pentyloxy)acetate;
3,3-dimethyl-5-oxo-5-(6-(3-(4-(trifluoromethyl)phenyl)ureido)hexylamino)pentanoic acid;
3,3-dimethyl-5-oxo-5-(6-(3-adamantylureido)hexylamino)pentanoic acid;
ethyl 8-(3-adamantylureido)octanoate; and
1-(8-methoxyoctyl)-3-adamantylurea,
or a stereoisomer, a tautomer, or a pharmaceutically acceptable salt thereof.

U.S. Provisional Application No. 61/017,380, filed on Dec. 28, 2007, and PCT Application No. PCT/US2008/______ with an Attorney Docket No. 074019-1910, entitled, “Soluble epoxide hydrolase inhibitors,” are incorporated herein by reference in their entirety.

In one embodiment, the compounds used in the methods of the present invention are selected from
1-(3,4-Difluoro-phenyl)-3-[1-(4-morpholin-4-yl-butyryl)-piperidin-4-yl]-urea;
1-(1-Acetyl-piperidin-4-yl)-3-(4-trifluoromethyl-phenyl)-urea;
1-(1-Methanesulfonyl-piperidin-4-yl)-3-(4-trifluoromethyl-phenyl)-urea;
1-[1-(3-Methyl-butyryl)-piperidin-4-yl]-3-(4-trifluoromethyl-phenyl)-urea;
1-(4-Fluoro-phenyl)-3-[1-(pyridine-3-carbonyl)-piperidin-4-yl]-urea;
1-[1-(Pyridine-3-carbonyl)-piperidin-4-yl]-3-(4-trifluoromethyl-phenyl)-urea;
1-[1-(Pyridine-2-carbonyl)-piperidin-4-yl]-3-(4-trifluoromethyl-phenyl)-urea;
4-{4-[3-(4-Fluoro-phenyl)-ureido]-piperidine-1-carbonyl}-benzoic acid;
4-{4-[3-(4-Trifluoromethyl-phenyl)-ureido]-piperidine-1-carbonyl}-benzoic acid;
1-(4-Fluoro-phenyl)-3-[1-(3-trifluoromethyl-benzenesulfonyl)-piperidin-4-yl]-urea;
1-(1-Benzenesulfonyl-piperidin-4-yl)-3-(4-fluoro-phenyl)-urea;
1-(4-Fluoro-phenyl)-3-[1-(4-trifluoromethyl-benzenesulfonyl)-piperidin-4-yl]-urea;
4-{4-[3-(4-Chloro-phenyl)-ureido]-piperidine-1-sulfonyl}-benzoic acid;
4-{4-[3-(4-Trifluoromethyl-phenyl)-ureido]-piperidine-1-sulfonyl}-benzoic acid;
1-(1-Benzenesulfonyl-piperidin-4-yl)-3-(4-trifluoromethyl-phenyl)-urea;
1-[1-(4-Chloro-benzenesulfonyl)-piperidin-4-yl]-3-(4-trifluoromethyl-phenyl)-urea;
1-[1-(4-Chloro-benzenesulfonyl)-piperidin-4-yl]-3-(4-fluoro-phenyl)-urea;
1-[1-(3-Trifluoromethyl-benzenesulfonyl)-piperidin-4-yl]-3-(4-trifluoromethyl-phenyl)-urea;
1-(1-Acetyl-piperidin-4-yl)-3-(4-fluoro-phenyl)-urea;
1-(1-Benzenesulfonyl-piperidin-4-yl)-3-(3-fluoro-phenyl)-urea;
1-[1-(4-Chloro-benzenesulfonyl)-piperidin-4-yl]-3-(3-fluoro-phenyl)-urea;
1-(1-Methanesulfonyl-piperidin-4-yl)-3-(3-trifluoromethyl-phenyl)-urea;
1-(1-Acetyl-piperidin-4-yl)-3-(3-trifluoromethyl-phenyl)-urea;
1-(1-Benzenesulfonyl-piperidin-4-yl)-3-(3-trifluoromethyl-phenyl)-urea;
1-(4-Fluoro-phenyl)-3-(1-methanesulfonyl-piperidin-4-yl)-urea;
1-(3-Fluoro-phenyl)-3-[1-(3-trifluoromethyl-benzenesulfonyl)-piperidin-4-yl]-urea;
1-[1-(4-Trifluoromethyl-benzenesulfonyl)-piperidin-4-yl]-3-(3-trifluoromethyl-phenyl)-urea;
1-[1-(4-Chloro-benzenesulfonyl)-piperidin-4-yl]-3-(3-trifluoromethyl-phenyl)-urea;
1-(3-Fluoro-phenyl)-3-(1-methanesulfonyl-piperidin-4-yl)-urea;
1-(1-Acetyl-piperidin-4-yl)-3-(3-fluoro-phenyl)-urea;
1-[1-(2-1H-Imidazol-4-yl-acetyl)-piperidin-4-yl]-3-(4-trifluoromethyl-phenyl)-urea;
1-(4-Chloro-phenyl)-3-[1-(2-1H-imidazol-4-yl-acetyl)-piperidin-4-yl]-urea;
1-[1-(1-Methyl-1H-imidazole-4-carbonyl)-piperidin-4-yl]-3-(4-trifluoromethyl-phenyl)-urea;
1-(4-Chlorophenyl)-3-(1-(4-morpholinobenzoyl)piperidin-4-yl)urea;
1-(1-(4-Morpholinobenzoyl)piperidin-4-yl)-3-(4-(trifluoromethyl)phenyl)urea;
Tert-butyl 2-methyl-2-(4-(4-(3-(4-(trifluoromethyl)phenyl)ureido)piperidine-1-carbonyl)phenoxy)propanoate;
1-(1-(2,5-Dimethyloxazole-4-carbonyl)piperidin-4-yl)-3-(4-(trifluoromethyl)phenyl)urea;
2-Methyl-2-(4-(4-(3-(4-(trifluoromethyl)phenyl)ureido)piperidine-1-carbonyl)phenoxy)propanoic acid;
1-(1-Pivaloylpiperidin-4-yl)-3-(4-(trifluoromethyl)phenyl)urea;
1-(1-(Isopropylsulfonyl)piperidin-4-yl)-3-(4-(trifluoromethyl)phenyl)urea;
1-(1-Acetyl-piperidin-3-yl)-3-adamantan-1-yl-urea;
1-Adamantan-1-yl-3-(1-methanesulfonyl-piperidin-3-yl)-urea;
1-Adamantan-1-yl-3-[1-(4-chloro-benzenesulfonyl)-piperidin-3-yl]-urea;
1-Adamantan-1-yl-3-[1-(3-trifluoromethyl-benzenesulfonyl)-piperidin-3-yl]-urea;
1-[1-(methylsulfonyl)piperidin-4-yl]-3-[4-(trifluoromethyl)phenyl]urea;
1-[1-(methylsulfonyl)piperidin-4-yl]-N′-(adamant-1-yl)urea;
1-(1-acetyl-piperidin-4-yl)-3-(1-adamantyl-methyl)-urea;
1-(1-acetylpiperidin-4-yl)-3-(cyclo-hexylmethyl)urea;
1-(1-acetylpiperidin-4-yl)-3-(4-(trifluoromethyl)benzyl)urea;
1-(1-acetylpiperidin-4-yl)-3-((tetrahydro-2H-pyran-4-yl)methyl)urea;
1-(1-acetylpiperidin-4-yl)-3-(3,4-dimethoxybenzyl)urea;
1-(1-acetylpiperidin-4-yl)-3-(8-hydroxyoctyl)urea;
1-(1-acetylpiperidin-4-yl)-3-(3,3-diphenylpropyl)urea;
methyl 4-((3-(1-acetylpiperidin-4-yl)ureido)methyl)benzoate;
1-(4-(trifluoromethyl)-phenyl)-3-(1-(5-(trifluoromethyl)-pyridin-2-yl)piperidin-4-yl)urea;
1-(4-(trifluoromethyl)-phenyl)-3-(1-(3-(trifluoromethyl)-pyridin-2-yl)piperidin-4-yl)urea;
1-(1-adamantyl)-3-(1-phenylpiperidin-4-yl)urea;
1-(1-adamantyl)-3-(1-(pyridin-4-yl)piperidin-4-yl)urea;
1-(1-phenylpiperidin-4-yl)-3-(4-(trifluoro-methyl)phenyl)urea;
2-(4-(3-(4-(trifluoro-methyl)phenyl)ureido)-piperidin-1-yl)nicotinamide;
2-(4-(3-(4-trifluoro-methylphenyl)ureido)-piperidin-1-yl)nicotinic acid;
1-(1-(thiazol-2-yl)piperidin-4-yl)-3-(4-(trifluoromethyl)phenyl)urea;
1-(1-phenylpiperidin-4-yl)-3-(4-(trifluoromethoxy)phenyl)urea;
1-(4-bromophenyl)-3-(1-phenylpiperidin-4-yl)urea;
1-(1-(4-fluorophenyl)piperidin-4-yl)-3-(4-(trifluoromethoxy)phenyl)urea;
1-adamantyl-3-(1-(2-fluorophenyl)piperidin-4-yl)urea;
1-(1-(2-fluorophenyl)piperidin-4-yl)-3-(4-(trifluoromethyl)phenyl)urea;
1-(1-acetylpiperidin-4-yl)-3-(3,5,7-trifluoroadamant-1-yl)urea;
1-(1-acetylpiperidin-4-yl)-3-(3-hydroxyadamant-1-yl)urea;
1-(1-acetylpiperidin-4-yl)-3-(3,5-difluoroadamant-1-yl)urea;
1-(1-acetylpiperidin-4-yl)-3-(3-fluoroadamant-1-yl)urea;
1-(1-acetylpiperidin-4-yl)-3-(4-hydroxyadamant-1-yl)urea;
1-(1-acetylpiperidin-4-yl)-3-(2-hydroxyadamant-1-yl)urea;
(R)-1-(1-acetylpiperidin-4-yl)-3-(4-hydroxyadamant-1-yl)urea;
(S)-1-(1-acetylpiperidin-4-yl)-3-(4-hydroxyadamant-1-yl)urea;
1-(1-acetylpiperidin-4-yl)-3-(4-oxoadamantyl)urea;
1-(1-acetylpiperidin-4-yl)-3-(4,4-difluoroadamantyl)urea;
1-(1-acetylpiperidin-4-yl)-3-(4-fluoroadamantyl)urea;
4-(4-(3-(4-(trifluoromethyl)phenyl)ureido)piperidine-1-carbonyl)benzene-sulfonamide;
4-(4-(3-(4-(trifluoromethoxy)-phenyl)ureido)-piperidine-1-carbonyl)benzenesulfonamide;
4-(4-(3-(1-adamantyl)ureido)-piperidine-1-carbonyl)benzene-sulfonamide;
3-(4-(3-(1-adamantyl)ureido)-piperidine-1-carbonyl)benzene-sulfonamide;
3-(4-(3-(1-adamantyl)ureido)-piperidine-1-carbonyl)-N-methylbenzene-sulfonamide;
3-(4-(3-(4-(trifluoromethyl)phenyl)ureido)piperidine-1-carbonyl)benzene-sulfonamide;
4-(4-(3-(4-(trifluoromethyl)phenyl)ureido)piperidine-1-carbonyl)-N-methylbenzene-sulfonamide;
4-(4-(3-(1-adamantyl)ureido)-piperidine-1-carbonyl)-N-methylbenzene-sulfonamide;
N-methyl-3-(4-(3-(4-(trifluoromethyl)phenyl)ureido)piperidine-1-carbonyl)benzene-sulfonamide;
2-(4-chlorophenyl)-N-(1-(3-(N-methyl-sulfamoyl)benzoyl)-piperidin-4-yl)acetamide;
N-methyl-3-(4-(3-(4-(trifluoromethoxy)-phenyl)ureido)-piperidine-1-carbonyl)benzene-sulfonamide;
4-(4-(3-(4-fluorophenyl)ureido)piperidine-1-carbonyl)-N-methylbenzene-sulfonamide;
tert-butyl 4-(3-(4-(morpholinosulfonyl)-phenyl)ureido)-piperidine-1-carboxylate;
1-(1-acetylpiperidin-4-yl)-3-(4-(morpholinosulfonyl)phenyl)urea;
tert-butyl 4-(3-quinolin-6-yl-ureido)piperidine-1-carboxylate;
tert-butyl 4-(3-1H-indol-6-yl-ureido)piperidine-1-carboxylate;
tert-butyl 4-(3-pyridin-4-yl-ureido)piperidine-1-carboxylate;
1-(1-acetylpiperidin-4-yl)-3-(quinolin-6-yl)urea;
tert-butyl 4-(3-(2,3-dihydro-1H-inden-5-yl)ureido)-piperidine-1-carboxylate;
1-(1-acetyl-piperidin-4-yl)-3-(2,3-dihydro-1H-inden-5-yl)urea;
1-(1-acetyl-piperidin-4-yl)-3-(pyridin-4-yl)urea;
tert-butyl 4-(3-(4-(1H-tetrazol-5-yl)phenyl)-ureido)piperidine-1-carboxylate;
1-(4-(1H-tetrazol-5-yl)phenyl)-3-(1-acetylpiperidin-4-yl)urea;
1-(1-acetylpiperidin-4-yl)-3-(pyridin-2-yl)urea;
1-(1-acetylpiperidin-4-yl)-3-(6-methoxypyridin-3-yl)urea;
1-(1-acetylpiperidin-4-yl)-3-(pyridin-3-yl)urea;
1-(6-methoxypyridin-3-yl)-3-(1-pivaloylpiperidin-4-yl)urea;
tert-butyl 4-(3-(2-methylbenzo[d]thiazol-6-yl)ureido)piperidine-1-carboxylate;
1-(1-acetylpiperidin-4-yl)-3-(2-methylbenzo[d]thiazol-6-yl)urea;
methyl 5-(3-(1-acetylpiperidin-4-yl)ureido)thiophene-2-carboxylate;
tert-butyl 4-(3-(5-(methoxycarbonyl)thiophen-2-yl)ureido)piperidine-1-carboxylate;
tert-butyl 4-(3-(5-(methoxycarbonyl)furan-2-yl)ureido)piperidine-1-carboxylate;
1-(1-acetylpiperidin-4-yl)-3-(2,3-dihydrobenzo[b][1,4]dioxin-6-yl)urea;
1-(1-adamantyl)-3-(1-(4-methoxyphenylsulfonyl)-piperidin-4-yl)urea;
1-(1-picolinoylpiperidin-4-yl)-3-(4-(trifluoro-methoxy)phenyl)urea;
1-(1-acetylpiperidin-4-yl)-3-(4-tert-butyl-cyclohexyl)urea;
1-(1-acetylpiperidin-4-yl)-3-(4-ethylcyclohexyl)urea;
1-(1-acetylpiperidin-4-yl)-3-(decahydronaphthalen-2-yl)urea;
1-(1-acetylpiperidin-4-yl)-3-(4,4-dimethyl-cyclohexyl)urea;
1-(1-acetylpiperidin-4-yl)-3-(bicyclo[2.2.1]heptan-2-yl)urea;
1-(1-adamantyl)-3-(1-(2,5-dimethyloxazole-4-carbonyl)piperidin-4-yl)urea;
tert-butyl 4-(3-(4-phenoxyphenyl)ureido)-piperidine-1-carboxylate;
tert-butyl 4-(3-(4-propoxyphenyl)ureido)-piperidine-1-carboxylate;
1-(1-acetylpiperidin-4-yl)-3-(4-propoxyphenyl)urea;
1-(1-acetylpiperidin-4-yl)-3-(4-phenoxyphenyl)urea;
1-(1-adamantyl)-3-(1-pivaloylpiperidin-4-yl)urea;
methyl 4-(3-(4-(trifluoromethyl)phenyl)ureido)piperidine-1-carboxylate;
ethyl 4-(3-(4-(trifluoromethyl)phenyl)ureido)piperidine-1-carboxylate;
N-(4-(trifluoromethyl)phenyl)-4-(3-(4-(trifluoro-methyl)phenyl)ureido)-piperidine-1-carboxamide;
tert-butyl 4-(3-cyclopentylureido)-piperidine-1-carboxylate;
1-(1-acetylpiperidin-4-yl)-3-cyclopentylurea;
1-(1-pivaloylpiperidin-4-yl)-3-(4-(trifluoro-methoxy)phenyl)urea;
isopropyl 4-(3-(4-(trifluoromethyl)phenyl)ureido)piperidine-1-carboxylate;
N,N-dimethyl-4-(3-(4-(trifluoromethyl)phenyl)-ureido)piperidine-1-carboxamide;
isopropyl 4-(3-(4-(trifluoromethoxy)phenyl)ureido)piperidine-1-carboxylate;
isopropyl 4-(3-(1-adamantyl)ureido)-piperidine-1-carboxylate;
1-(1-(biphenyl-4-ylsulfonyl)piperidin-4-yl)-3-adamantylurea;
1-adamantyl-3-(1-(naphthalen-262-ylsulfonyl)piperidin-4-yl)urea;
1-adamantyl-3-(1-(phenylsulfonyl)piperidin-4-yl)urea;
1-(1-(4-chlorophenylsulfonyl)piperidin-4-yl)-3-cyclohexylurea;
1-adamantyl-3-(1-(thiophen-2-ylsulfonyl)piperidin-4-yl)urea;
1-(1-(benzylsulfonyl)piperidin-4-yl)-3-adamantylurea;
1-(1-(4-tert-butylphenylsulfonyl)piperidin-4-yl)-3-adamantylurea;
1-cyclohexyl-3-(1-propionylpiperidin-4-yl)urea;
1-adamantyl-3-(1-(2-(trifluoromethyl)phenylsulfonyl)piperidin-4-yl)urea;
1-adamantyl-3-(1-(o-tolylsulfonyl)piperidin-4-yl)urea;
1-(1-(3-chloro-2-methylphenylsulfonyl)piperidin-4-yl)-3-adamantylurea;
1-(1-(2-chloro-6-methylphenylsulfonyl)piperidin-4-yl)-3-adamantylurea;
1-adamantyl-3-(1-(4-(trifluoromethyl)phenylsulfonyl)piperidin-4-yl)urea;
1-cyclohexyl-3-(1-(3,4-dichlorophenylsulfonyl)piperidin-4-yl)urea;
1-adamantyl-3-(1-(3-(trifluoromethyl)phenylsulfonyl)piperidin-4-yl)urea;
1-adamantyl-3-(1-(1-methyl-1H-imidazole-4-carbonyl)piperidin-4-yl)urea;
1-cyclohexyl-3-(1-picolinoylpiperidin-4-yl)urea;
1-adamantyl-3-(1-(4-(methylsulfonyl)phenylsulfonyl)piperidin-4-yl)urea;
1-(1-(4-chlorophenylsulfonyl)piperidin-4-yl)-3-cyclohexylurea;
1-(1-acetylpiperidin-4-yl)-3-cyclohexylurea;
1-cyclohexyl-3-(1-(3-(trifluoromethyl)phenylsulfonyl)piperidin-4-yl)urea;
4-(4-(3-adamantylureido)piperidin-1-ylsulfonyl)benzoic acid;
1-(1-(4-chlorobenzoyl)piperidin-4-yl)-3-adamantylurea;
tert-butyl 4-(3-(4-(trifluoromethyl)phenyl)ureido)piperidine-1-carboxylate;
tert-butyl 4-(3-cycloheptylureido)piperidine-1-carboxylate;
tert-butyl 4-(3-(4-(methylsulfonyl)phenyl)ureido)piperidine-1-carboxylate;
tert-butyl 4-(3-cyclobutylureido)piperidine-1-carboxylate;
tert-butyl 4-(3-(4-bromophenyl)ureido)piperidine-1-carboxylate;
1-(1-acetylpiperidin-4-yl)-3-(4-(dimethylamino)phenyl)urea;
4-(3-(1-acetylpiperidin-4-yl)ureido)benzoic acid;
4-(3-(1-(tert-butoxycarbonyl)piperidin-4-yl)ureido)benzoic acid;
1-(1-(isopropylsulfonyl)piperidin-4-yl)-3-(4-(trifluoromethoxy)phenyl)urea;
N-adamantyl-4-(3-adamantylureido)piperidine-1-carboxamide;
N-(1-acetylpiperidin-4-yl)-4-(3-adamantylureido)piperidine-1-carboxamide;
1-(1-acetylpiperidin-4-yl)-3-(4-methylbicyclo[2.2.2]octan-1-yl)urea;
1-adamantyl-3-(1-(3-hydroxypropanoyl)piperidin-4-yl)urea;
1-(1-acetylpiperidin-4-yl)-3-(4-(methylsulfonyl)phenyl)urea;
1-cyclohexyl-3-(1-(4-morpholinobutanoyl)piperidin-4-yl)urea;
1-(1-acetylpiperidin-4-yl)-3-(4,4-difluorocyclohexyl)urea;
1-(1-acetylpiperidin-4-yl)-3-cyclobutylurea;
tert-butyl 4-(3-cyclooctylureido)piperidine-1-carboxylate;
tert-butyl 4-(3-(4-(dimethylamino)phenyl)ureido)piperidine-1-carboxylate;
1,1′-(1,1′-carbonylbis(piperidine-4,1-diyl))bis(3-adamantylurea);
tert-butyl 4-(3-(4-(methoxycarbonyl)phenyl)ureido)piperidine-1-carboxylate;
tert-butyl 4-(3-(4-(pyrrolidin-1-ylmethyl)phenyl)ureido)piperidine-1-carboxylate;
methyl 4-(3-(1-acetylpiperidin-4-yl)ureido)benzoate;
1-(4-(methylsulfonyl)phenyl)-3-(1-pivaloylpiperidin-4-yl)urea;
1-(1-(4-hydroxybutanoyl)piperidin-4-yl)-3-(4-(trifluoromethoxy)phenyl)urea;
1-adamantyl-3-(1-(3,3-dimethylbutanoyl)piperidin-4-yl)urea;
1-adamantyl-3-(1-(4-hydroxybutanoyl)piperidin-4-yl)urea;
1-adamantyl-3-(1-(3-hydroxypropylsulfonyl)piperidin-4-yl)urea;
1-(1-(3-hydroxypropylsulfonyl)piperidin-4-yl)-3-(4-(trifluoromethoxy)phenyl)urea;
1-adamantyl-3-(1-(2-methoxyacetyl)piperidin-4-yl)urea;
1-(1-(tert-butylsulfonyl)piperidin-4-yl)-3-(4-(trifluoromethoxy)phenyl)urea;
1-(1-(tert-butylsulfonyl)piperidin-4-yl)-3-adamantylurea;
1-(1-(morpholine-4-carbonyl)piperidin-4-yl)-3-(4-(trifluoromethoxy)phenyl)urea;
1-(1-acetylpiperidin-4-yl)-3-(4-cyanophenyl)urea;
1-(4-cyanophenyl)-3-(1-pivaloylpiperidin-4-yl)urea;
1-adamantyl-3-(1-(morpholine-4-carbonyl)piperidin-4-yl)urea;
1-(1-acetylpiperidin-4-yl)-3-(spiro[4.5]decan-8-yl)urea;
1-(1-acetylpiperidin-4-yl)-3-cyclooctylurea;
2-(4-chlorophenyl)-N-(1-(3-(N-methyl-sulfamoyl)benzoyl)-piperidin-4-yl)acetamide;
tert-butyl 4-(3-(4-morpholinophenyl)ureido)piperidine-1-carboxylate;
1-(1-acetylpiperidin-4-yl)-3-(4-morpholinophenyl)urea;
1-(1-acetylpiperidin-4-yl)-3-(adamantyl)urea;
1-(1-(pyridin-3-ylsulfonyl)piperidin-4-yl)-3-(4-(trifluoromethoxy)phenyl)urea;
1-(1-nicotinoylpiperidin-4-yl)-3-(4-(trifluoromethoxy)phenyl)urea;
1-(adamantyl)-3-(1-picolinoylpiperidin-4-yl)urea;
1-adamantyl-3-(5-(2-(2-ethoxyethoxy)ethoxy)pentyl)urea;
1-adamantyl-3-(8-hydroxyoctyl)urea;
1-(1-(3,3-dimethylbutanoyl)piperidin-4-yl)-3-(4-(trifluoromethyl)phenyl)urea;
1-(6-phenoxypyridin-3-yl)-3-(4-(trifluoromethyl)phenyl)urea;
1-(4-(phenylsulfonyl)phenyl)-3-(4-(trifluoromethyl)phenyl)urea;
4-(4-(3-(adamantyl)ureido)phenoxy)benzoic acid;
4-(4-(3-(adamantyl)ureido)cyclohexyloxy)benzoic acid; and
1-(3-(morpholine-4-carbonyl)phenyl)-3-(4-(trifluoromethyl)phenyl)urea;
or a stereoisomer, a tautomer, or a pharmaceutically acceptable salt thereof.

In one embodiment, the compound of Formula I-VII is a soluble epoxide hydrolase inhibitor having an IC₅₀ value of less than 25 μM. In one embodiment, the compound of Formula I-VII has an IC₅₀ value of less than 10 μM. In one embodiment, the compound of Formula I-VII has an IC₅₀ value of less than 1 μM.

In some embodiments, the compound to be administered is a compound, stereoisomer, tautomer, or a pharmaceutically acceptable salt thereof where the compound is selected from Table 1-Table 12, as provided below.

TABLE 1

| Comp. | | |
|---|---|---|
| No. | Compound structure | Compound name |
| 1 | 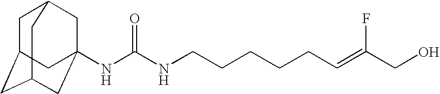 | (Z)-1-(7-fluoro-8- hydroyxoct-6- enyl)-3- (adamantyl)urea |
| 2 | 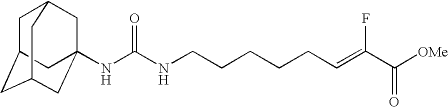 | (Z)-methyl 2- fluoro-8-(3- adamantylureido)oct- 2-enoate |
| 3 | 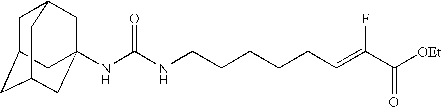 | (Z)-ethyl 2-fluoro- 8-(3- adamantylureido)oct- 2-enoate |
| 4 | 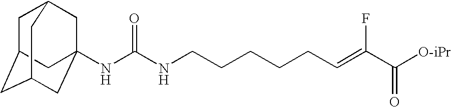 | (Z)-isopropyl 2- fluoro-8-(3- adamantylureido)oct- 2-enoate |
| 5 | 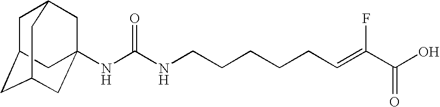 | (Z)-2-fluoro-8-(3- adamantylureido)oct- 2-enoic acid |
| 6 | 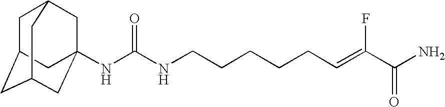 | (Z)-2-fluoro-8-(3- adamantylureido)oct- 2-enamide |
| 7 | 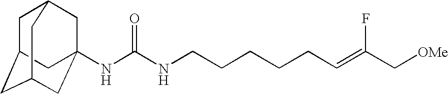 | (Z)-1-(7-fluoro-8- methoxyoct-6- enyl)-3- adamantylurea |
| 8 | 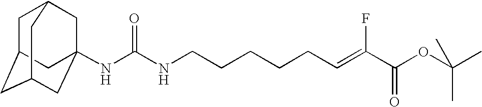 | (Z)-t-butyl 2- fluoro-8-(3- adamantylureido)oct- 2-enoate |
| 9 | 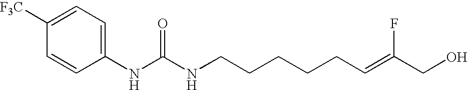 | (Z)-1-(7-fluoro-8- hydroxyoct-6- enyl)-3-(4- (trifluoromethyl)phe- nyl)urea |
| 10 | 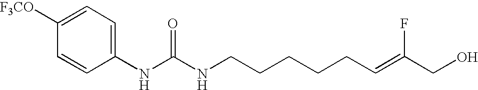 | (Z)-1-(7-fluoro-8- hydroxyoct-6- enyl)-3-(4- (trifluoromethoxy)phe- nyl)urea |
| 11 | 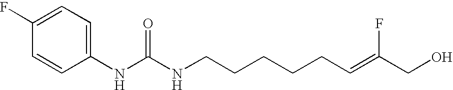 | (Z)-1-(7-fluoro-8- hydroxyoct-6- enyl)-3-(4- fluorophenyl)urea |
| 12 | 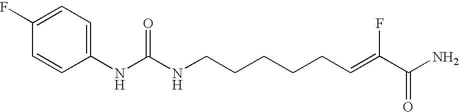 | (Z)-2-fluoro-8-(3- (4- fluorophenyl)ureido) oct-2-enamide |
| 13 | 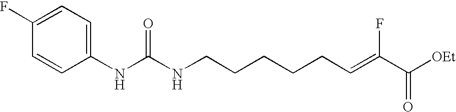 | (Z)-ethyl 2-fluoro- 8-(3-(4- fluorophenyl)ureido) oct-2-enoate |
| 14 | 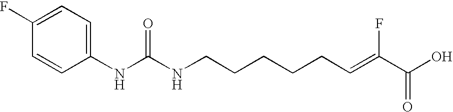 | (Z)-2-fluoro-8-(3- (4- fluorophenyl)ureido) oct-2-enoic acid |
| 15 | 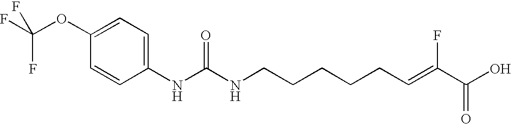 | (Z)-2-fluoro-8-(3- (4- (trifluoromethoxy)phe- nyl)ureido)oct-2- enoic acid |
| 16 | 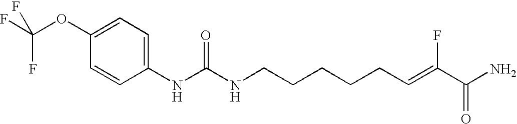 | (Z)-2-fluoro-8-(3- (4- (trifluoromethoxy)phe- nyl)ureido)oct-2- enamide |
| 17 | 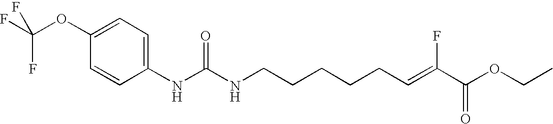 | (Z)-ethyl 2-fluoro- 8-(3-(4- (trifluoromethoxy)phe- nyl)ureido)oct-2- enoate |
| 18 | 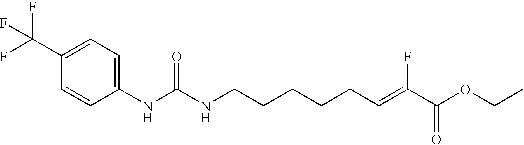 | (Z)-ethyl 2-fluoro- 8-(3-(4- (trifluoromethyl)phe- nyl)ureido)oct-2- enoate |
| 19 | 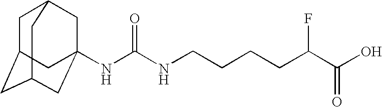 | 2-fluoro-6-(3- adamantylureido)hex- anoic acid |
| 20 | 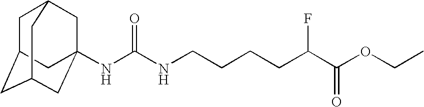 | Ethyl 2-fluoro-6-(3- adamantylureido)hex- anoate |
| 21 | 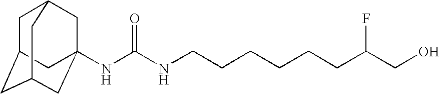 | 1-(7-fluoro-8- hydroxyoctyl)-3- (adamantyl)urea |
| 22 | 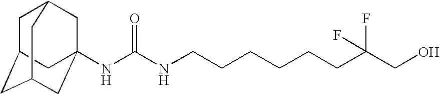 | 1-(7,7-difluoro-8- hydroxyoctyl)-3- (adamantyl)urea |
| 23 | 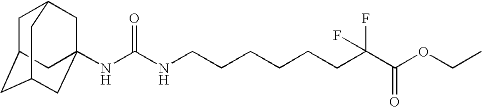 | ethyl 2,2-difluoro- 8-(3- adamantylureido)octa- noate |
| 24 | 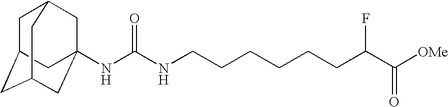 | methyl 2-fluoro-8- (3- adamantylureido)octa- noate |
| 25 | 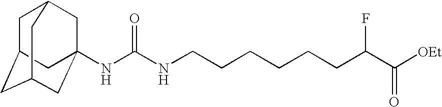 | ethyl 2-fluoro-8-(3- adamantylureido)octa- noate |
| 26 | 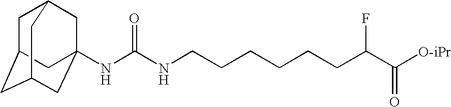 | isopropyl 2-fluoro- 8-(3- adamantylureido)octa- noate |
| 27 | 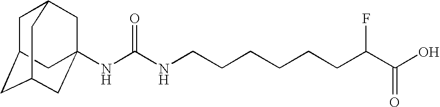 | 2-fluoro-8-(3- adamantylureido)octa- noic acid |
| 28 | 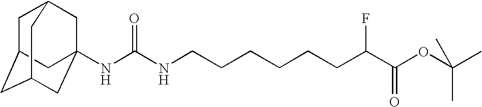 | t-butyl 2-fluoro-8- (3- adamantylureido)octa- noate |
| 29 | 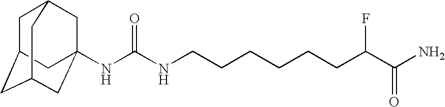 | 2-fluoro-8-(3- adamantylureido)octa- namide |
| 30 | 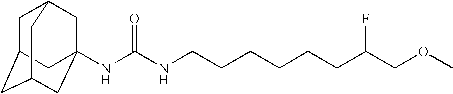 | 1-(7-fluoro-8- methoxyoctane)-3- adamantylurea |
| 31 | 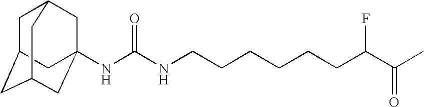 | 1-(7-fluoro-8- oxononyl)-3- adamantylurea |
| 32 | 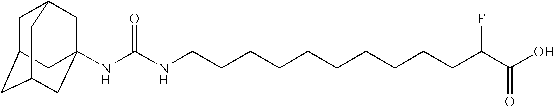 | 2-fluroo-12-(3- adamantylureido)dode- canoic acid |
| 33 | 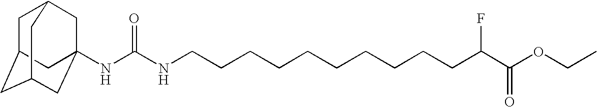 | Ethyl 2-fluoro-12- (3- adamantylureido)dode- cenoate |
| 34 | 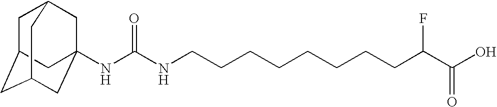 | 2-fluoro-10-(3- adamantylureido)deca- noic acid |
| 35 | 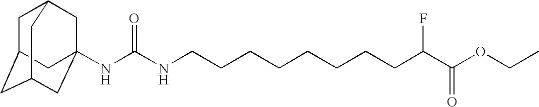 | Ethyl 2-fluoro-10- (3- adamantylureido)deca- noate |
| 36 | 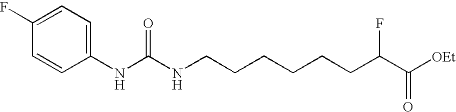 | ethyl 2-fluoro-8-(3- (4- fluorophenyl)ureido) octanoate |
| 37 | 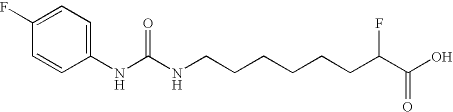 | 2-fluoro-8-(3-(4- fluroophenyl)ureido) octanoic acid |
| 38 | 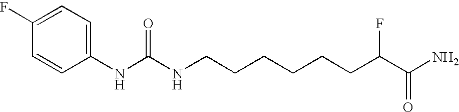 | 2-fluoro-8-(3-(4- fluorophenyl)ureido) octanamide |
| 39 | 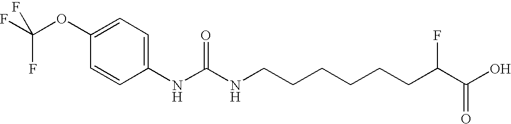 | 2-fluoro-8-(3-(4- (trifluoromethoxy)phe- nyl)ureido)octanoic acid |
| 40 | 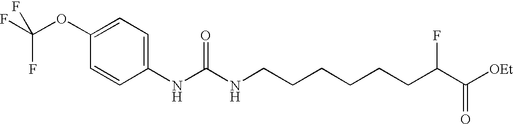 | ethyl 2-fluoro-8-(3- (4- (trifluoromethoxy)phe- nyl)ureido)octanoate |
| 41 | 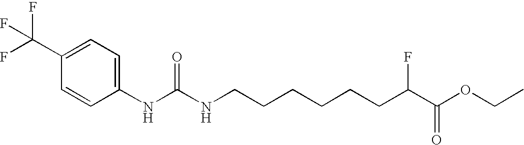 | ethyl 2-fluoro-8-(3- (4- (trifluoromethyl)phe- nyl)ureido)octanoate |
| 42 | 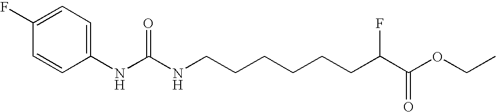 | ethyl 2-fluoro-8-(3- (4- fluorophenyl)ureido) octanoate |
| 43 | 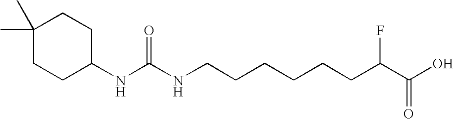 | 8-(3-(4,4- dimethylcyclohexyl) ureido)-2- fluorooctanoic acid |
| 44 | 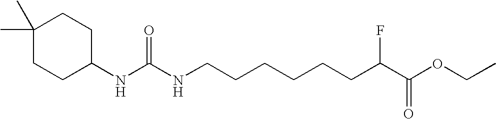 | ethyl 8-(3-(4,4- dimethylcyclohexyl) ureido)-2- fluorooctanoate |
| 45 | 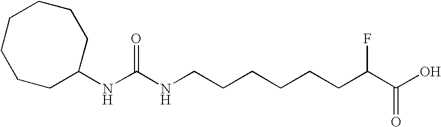 | 8-(3- cyclooctylureido)- 2-fluorooctanoic acid |
| 45a | 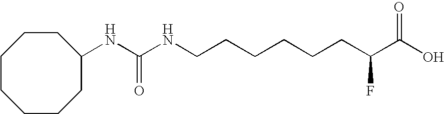 | (S)-8-(3- cyclooctylureido)- 2-fluorooctanoic acid |
| 45b | 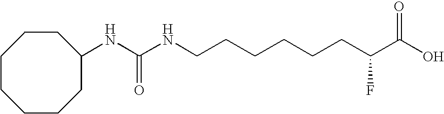 | (R)-8-(3- cyclooctylureido)- 2-fluorooctanoic acid |
| 46 | 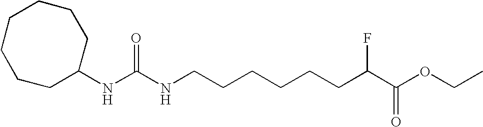 | ethyl 8-(3- cyclooctylureido)- 2-fluorooctanoate |
| 47 | 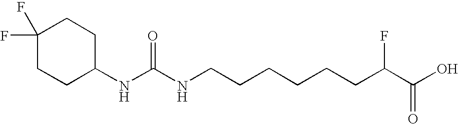 | 8-(3-(4,4- difluorocyclohexyl) ureido)-2- fluorooctanoic acid |
| 48 | 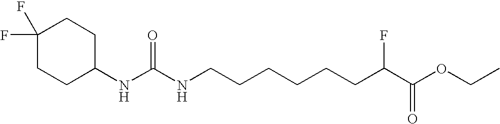 | ethyl 8-(3-(4,4- difluorocyclohexyl) ureido)-2- fluorooctanoate |
| 49 | 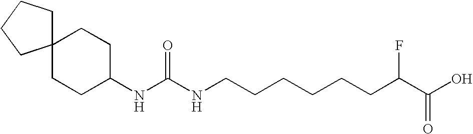 | 2-fluoro-8-(3- spiro[4.5]decan-8- ylureido)octanoic acid |
| 50 | 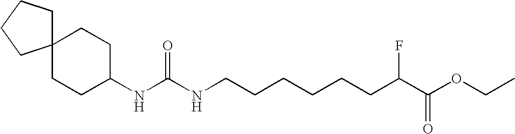 | ethyl 2-fluoro-8-(3- spiro[4.5]decan-8- ylureido)octanoate |
| 51 | 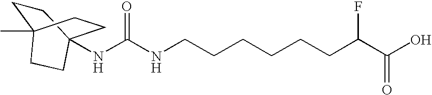 | 2-fluoro-8-(3-(4- methylbicyclo[2.2.2] octan-1- yl)ureido)octanoic acid |
| 52 | 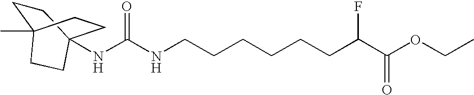 | ethyl 2-fluoro-8-(3- (4- methylbicyclo[2.2.2] octan-1- yl)ureido)octanoate |
| 53 | 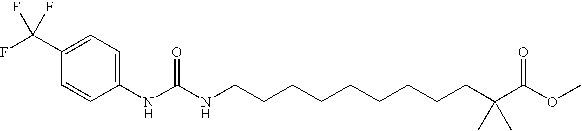 | methyl 2,2- dimethyl-11-(3-(4- (trifluoromethyl)phe- nyl)ureido)undeca noate |
| 54 | 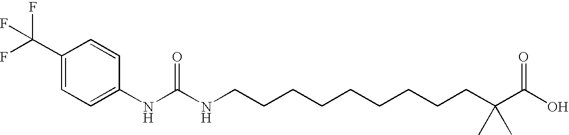 | 2,2-dimethyl-11-(3- (4- (trifluoromethyl)phe- nyl)ureido)undeca noic acid |
| 55 | 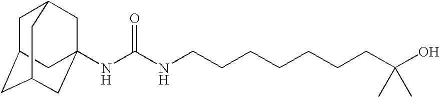 | 1-(8-hydroxy-8- methylnonyl)-3- adamantylurea |
| 56 | 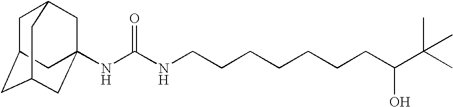 | 1-(8-hydroxy-9,9- dimethyldecyl)-3- adamantylurea |
| 57 | 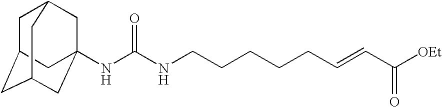 | (E)-ethyl 8-(3- adamantylureido)oct- 2-enoate |
| 58 | 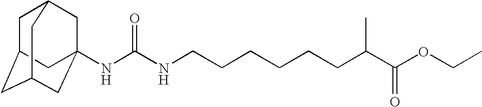 | ethyl 2-methyl-8- (3- adamantylureido)octa- noate |
| 59 | 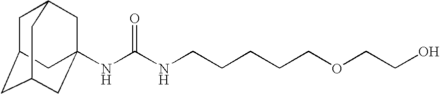 | 1-(5-(2- hydroxyethoxy)pen tyl)-3- adamantylurea |
| 60 | 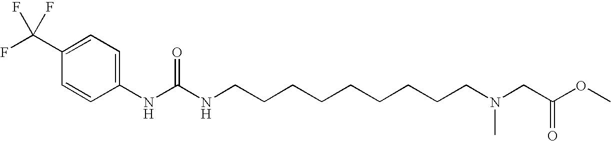 | methyl 2- (methyl(9-(3-(4- (trifluoromethyl)phe- nyl)ureido)nonyl)a mino)acetate |
| 61 | 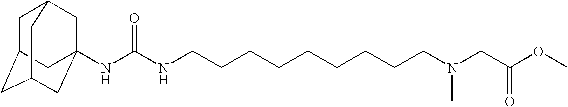 | methyl 2- (methyl(9-(3- adamantylureido)no nyl)amino)acetate |
| 62 | 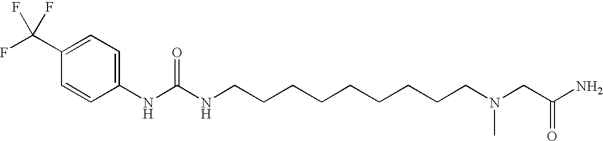 | 2-(methyl(9-(3-(4- (trifluoromethyl)phe- nyl)ureido)nonyl)a mino)acetamide |
| 63 | 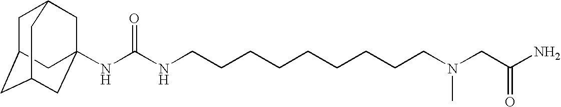 | 2-(methyl(9-(3- adamantylureido)no nyl)amino)acetamide |
| 64 | 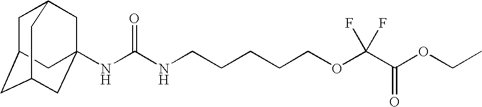 | ethyl 2,2-difluoro- 2-(5-(3- adamantylureido)pentyl- oxy)acetate |
| 65 | 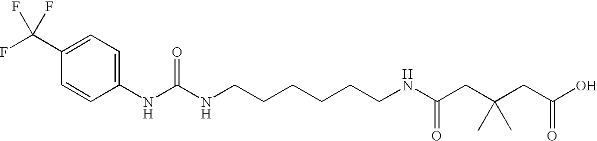 | 3,3-dimethyl-5- oxo-5-(6-(3-(4- (trifluoromethyl)phe- nyl)ureido)hexyla mino)pentanoic acid |
| 66 | 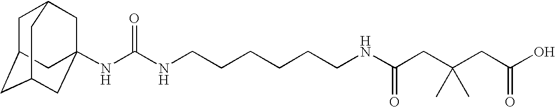 | 3,3-dimethyl-5- oxo-5-(6-(3- adamantylureido)hexyl- amino)pentanoic acid |
| 67 | 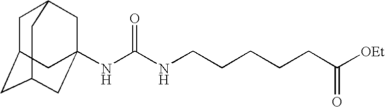 | ethyl 8-(3- adamantylureido)octa- noate |
| 68 | 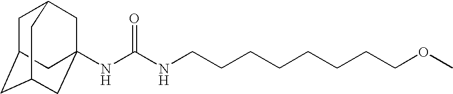 | 1-(8- methoxyoctyl)-3- adamantylurea |

TABLE 2

| Compound No. | Structure | Name |
|---|---|---|
| 1 | 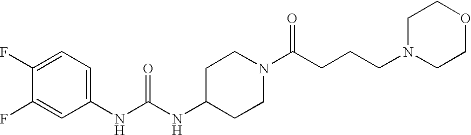 | 1-(3,4-Difluoro-phenyl)-3-[1- (4-morpholin-4-yl-butyryl)- piperidin-4-yl]-urea |
| 2 | 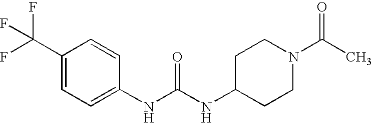 | 1-(1-Acetyl-piperidin-4-yl)-3- (4-trifluoromethyl-phenyl)- urea |
| 3 | 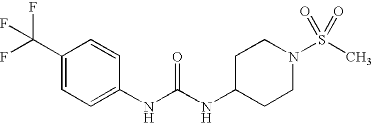 | 1-(1-Methanesulfonyl- piperidin-4-yl)-3-(4- trifluoromethyl-phenyl)-urea |
| 4 | 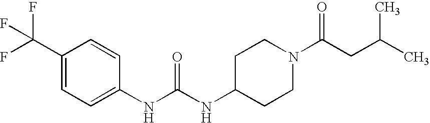 | 1-[1-(3-Methyl-butyryl)- piperidin-4-yl]-3-(4- trifluoromethyl-phenyl)-urea |
| 5 | 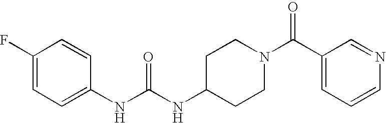 | 1-(4-Fluoro-phenyl)-3-[1- (pyridine-3-carbonyl)- piperidin-4-yl]-urea |
| 6 | 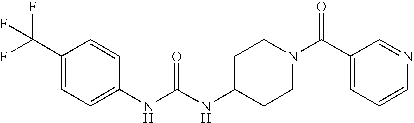 | 1-[1-(Pyridine-3-carbonyl)- piperidin-4-yl]-3-(4- trifluoromethyl-phenyl)-urea |
| 7 | 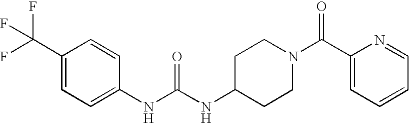 | 1-[1-(Pyridine-2-carbonyl)- piperidin-4-yl]-3-(4- trifluoromethyl-phenyl)-urea |
| 8 | 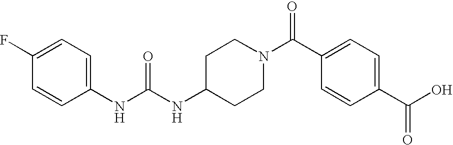 | 4-{4-[3-(4-Fluoro-phenyl)- ureido]-piperidine-1- carbonyl}-benzoic acid |
| 9 | 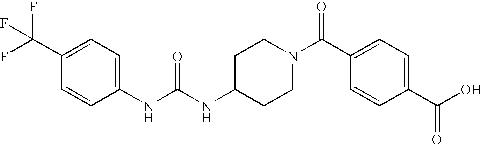 | 4-{4-[3-(4-Trifluoromethyl- phenyl)-ureido]-piperidine-1- carbonyl}-benzoic acid |
| 10 | 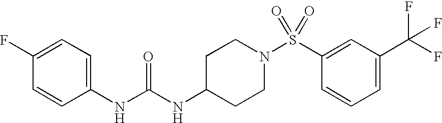 | 1-(4-Fluoro-phenyl)-3-[1-(3- trifluoromethyl- benzenesulfonyl)-piperidin-4- yl]-urea |
| 11 | 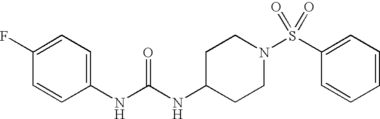 | 1-(1-Benzenesulfonyl- piperidin-4-yl)-3-(4-fluoro- phenyl)-urea |
| 12 | 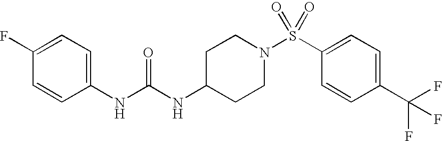 | 1-(4-Fluoro-phenyl)-3-[1-(4- trifluoromethyl- benzenesulfonyl)-piperidin-4- yl]-urea |
| 13 | 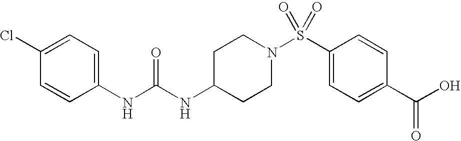 | 4-{4-[3-(4-Chloro-phenyl)- ureido]-piperidine-1- sulfonyl}-benzoic acid |
| 14 | 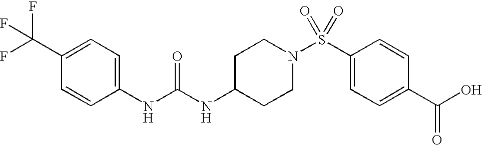 | 4-{4-[3-(4-Trifluoromethyl- phenyl)-ureido]-piperidine-1- sulfonyl}-benzoic acid |
| 15 | 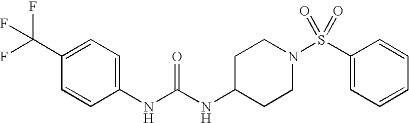 | 1-(1-Benzenesulfonyl- piperidin-4-yl)-3-(4- trifluoromethyl-phenyl)-urea |
| 16 | 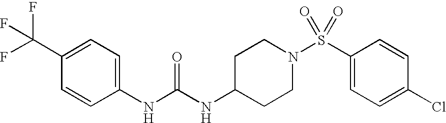 | 1-[1-(4-Chloro- benzenesulfonyl)-piperidin-4- yl]-3-(4-trifluoromethyl- phenyl)-urea |
| 17 | 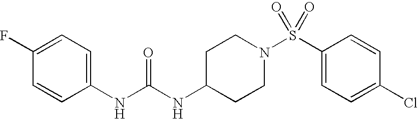 | 1-[1-(4-Chloro- benzenesulfonyl)-piperidin-4- yl]-3-(4-fluoro-phenyl)-urea |
| 18 | 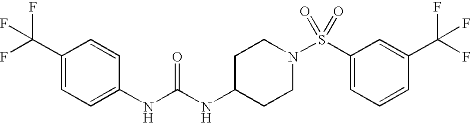 | 1-[1-(3-Trifluoromethyl- benzenesulfonyl)-piperidin-4- yl]-3-(4-trifluoromethyl- phenyl)-urea |
| 19 | 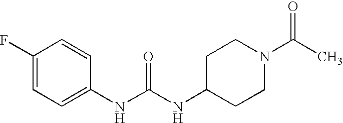 | 1-(1-Acetyl-piperidin-4-yl)-3- (4-fluoro-phenyl)-urea |
| 20 | 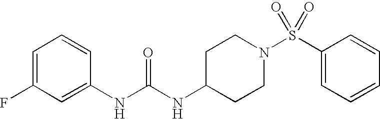 | 1-(1-Benzenesulfonyl- piperidin-4-yl)-3-(3-fluoro- phenyl)-urea |
| 21 | 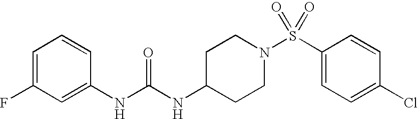 | 1-[1-(4-Chloro- benzenesulfonyl)-piperidin-4- yl]-3-(3-fluoro-phenyl)-urea |
| 22 | 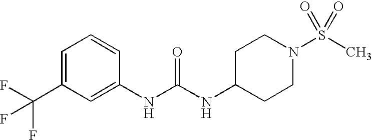 | 1-(1-Methanesulfonyl- piperidin-4-yl)-3-(3- trifluoromethyl-phenyl)-urea |
| 23 | 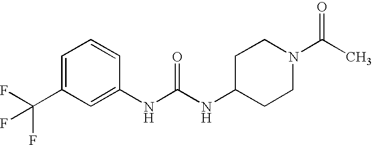 | 1-(1-Acetyl-piperidin-4-yl)-3- (3-trifluoromethyl-phenyl)- urea |
| 24 | 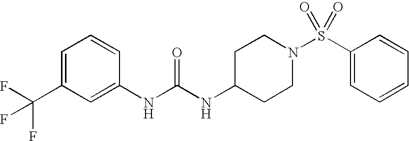 | 1-(1-Benzenesulfonyl- piperidin-4-yl)-3-(3- trifluoromethyl-phenyl)-urea |
| 25 | 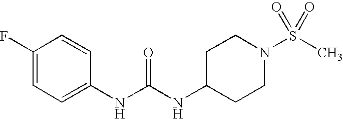 | 1-(4-Fluoro-phenyl)-3-(1- methanesulfonyl-piperidin-4- yl)-urea |
| 26 | 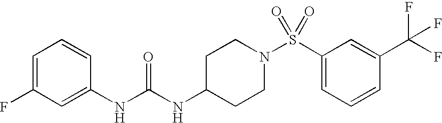 | 1-(3-Fluoro-phenyl)-3-[1-(3- trifluoromethyl- benzenesulfonyl)-piperidin-4- yl]-urea |
| 27 | 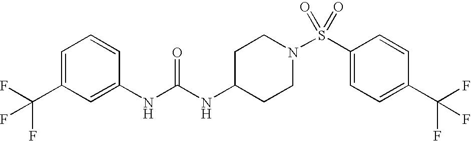 | 1-[1-(4-Trifluoromethyl- benzenesulfonyl)-piperidin-4- yl]-3-(3-trifluoromethyl- phenyl)-urea |
| 28 | 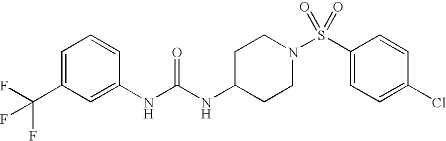 | 1-[1-(4-Chloro- benzenesulfonyl)-piperidin-4- yl]-3-(3-trifluoromethyl- phenyl)-urea |
| 29 | 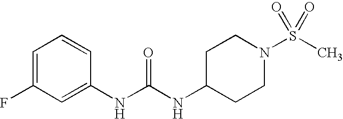 | 1-(3-Fluoro-phenyl)-3-(1- methanesulfonyl-piperidin-4- yl)-urea |
| 30 | 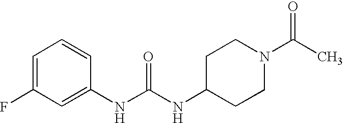 | 1-(1-Acetyl-piperidin-4-yl)-3- (3-fluoro-phenyl)-urea |
| 31 | 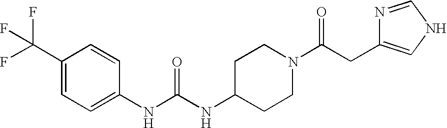 | 1-[1-(2-1H-Imidazol-4-yl- acetyl)-piperidin-4-yl]-3-(4- trifluoromethyl-phenyl)-urea |
| 32 | 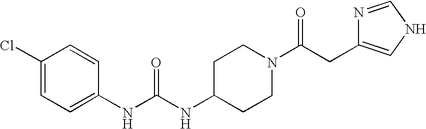 | 1-(4-Chloro-phenyl)-3-[1-(2- 1H-imidazol-4-yl-acetyl)- piperidin-4-yl]-urea |
| 33 | 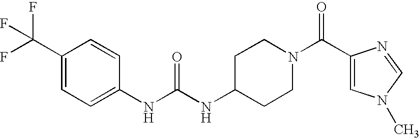 | 1-[1-(1-Methyl-1H-imidazole- 4-carbonyl)-piperidin-4-yl]-3- (4-trifluoromethyl-phenyl)-urea |

TABLE 3

| Compound | | |
|---|---|---|
| No. | Structure | Name |
| 1 | 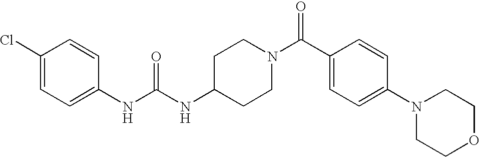 | 1-(4-Chlorophenyl)-3-(1-(4- morpholinobenzoyl)piperidin-4- yl)urea |
| 2 | 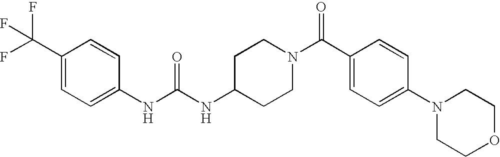 | 1-(1-(4- Morpholinobenzoyl)piperidin- 4-yl)-3-(4- (trifluoromethyl)phenyl)urea |
| 3 | 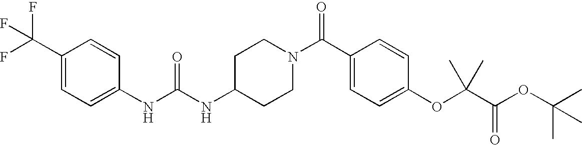 | Tert-butyl 2-methyl-2-(4-(4-(3- (4- (trifluoromethyl)phenyl)ureido) piperidine-1- carbonyl)phenoxy)propanoate |
| 4 | 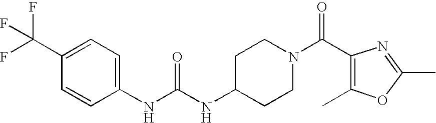 | 1-(1-(2,5-Dimethyloxazole-4- carbonyl)piperidin-4-yl)-3-(4- (trifluoromethyl)phenyl)urea |
| 5 | 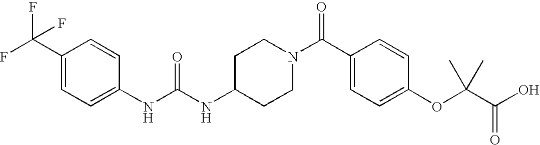 | 2-Methyl-2-(4-(4-(3-(4- (trifluoromethyl)phenyl)ureido) piperidine-1- carbonyl)phenoxy)propanoic acid |
| 6 | 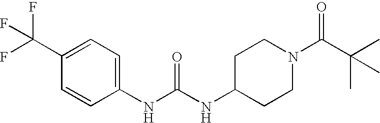 | 1-(1-Pivaloylpiperidin-4-yl)-3- (4-(trifluoromethyl)phenyl)urea |
| 7 | 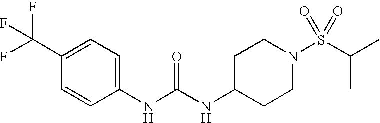 | 1-(1- (Isopropylsulfonyl)piperidin-4- yl)-3-(4- (trifluoromethyl)phenyl)urea |

TABLE 4

| Compound | | |
|---|---|---|
| No. | Structure | Name |
| 1 | 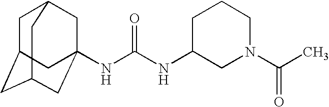 | 1-(1-Acetyl-piperidin-3-yl)-3- adamantan-1-yl-urea |
| 2 | 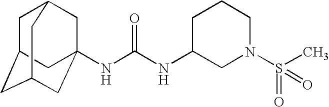 | 1-Adamantan-1-yl-3-(1- methanesulfonyl-piperidin-3-yl)- urea |
| 3 | 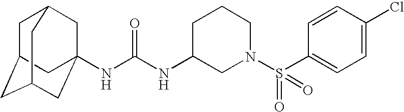 | 1-Adamantan-1-yl-3-[1-(4-chloro- benzenesulfonyl)-piperidin-3-yl]- urea |
| 4 | 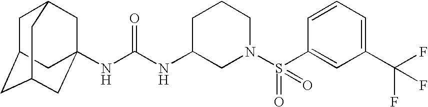 | 1-Adamantan-1-yl-3-[1-(3- trifluoromethyl-benzenesulfonyl)- piperidin-3-yl]-urea |

TABLE 5

| Compound | | |
|---|---|---|
| No. | Structure | Name |
| 1 | 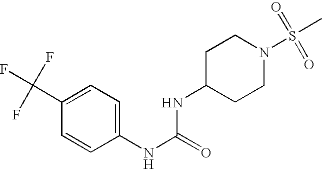 | 1-[1-(methylsulfonyl)piperidin-4- yl]-3-[4-(trifluoromethyl)phenyl] urea |
| 2 | 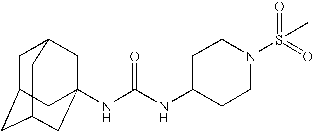 | 1-[1-(methylsulfonyl)piperidin-4- yl]-N′-(adamant-1-yl)urea |

TABLE 6

| Compound | | |
|---|---|---|
| No. | Structure | Name |
| 1 | 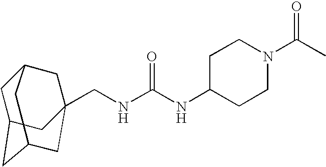 | 1-(1-acetyl-piperidin-4-yl)-3- (1-adamantyl-methyl)-urea |
| 2 | 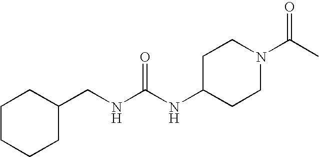 | 1-(1-acetylpiperidin-4-yl)-3- (cyclo-hexylmethyl)urea |
| 3 | 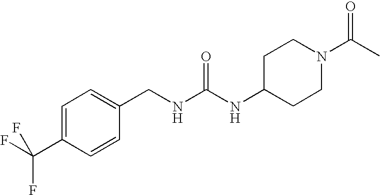 | 1-(1-acetylpiperidin-4-yl)-3- (4- (trifluoromethyl)benzyl)urea |
| 4 | 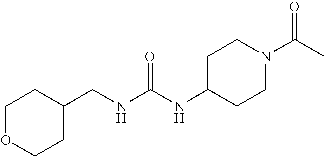 | 1-(1-acetylpiperidin-4-yl)-3- ((tetrahydro-2H-pyran-4- yl)methyl)urea |
| 5 | 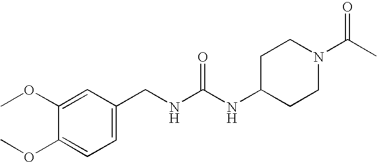 | 1-(1-acetylpiperidin-4-yl)-3- (3,4-dimethoxybenzyl)urea |
| 6 | 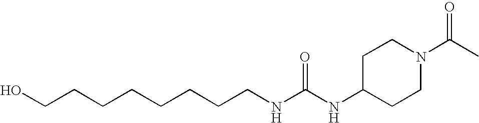 | 1-(1-acetylpiperidin-4-yl)-3- (8-hydroxyoctyl)urea |
| 7 | 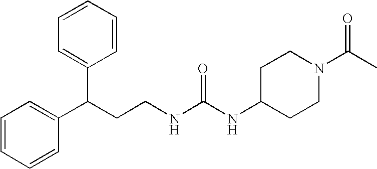 | 1-(1-acetylpiperidin-4-yl)-3- (3,3-diphenylpropyl)urea |
| 8 | 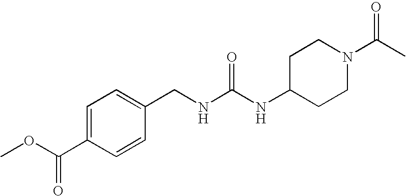 | methyl 4-((3-(1- acetylpiperidin-4- yl)ureido)methyl)benzoate |

TABLE 7

| Compound | | |
|---|---|---|
| No. | Structure | Name |
| 1 | 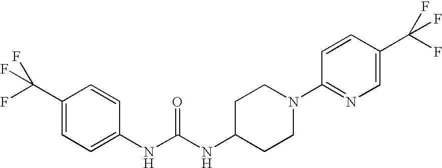 | 1-(4-(trifluoromethyl)- phenyl)-3-(1-(5- (trifluoromethyl)-pyridin-2- yl)piperidin-4-yl)urea |
| 2 | 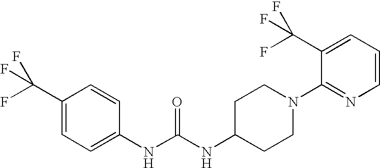 | 1-(4-(trifluoromethyl)- phenyl)-3-(1-(3- (trifluoromethyl)-pyridin-2- yl)piperidin-4-yl)urea |
| 3 | 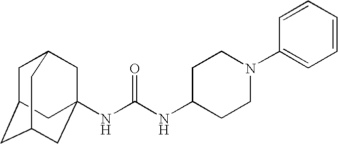 | 1-(1-adamantyl)-3-(1- phenylpiperidin-4-yl)urea |
| 4 | 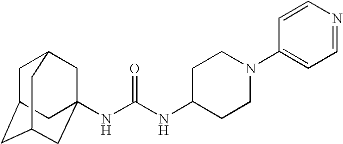 | 1-(1-adamantyl)-3-(1- (pyridin-4-yl)piperidin-4- yl)urea |
| 5 | 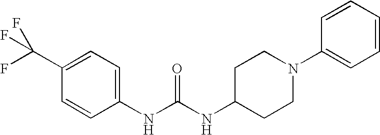 | 1-(1-phenylpiperidin-4-yl)-3- (4-(trifluoro- methyl)phenyl)urea |
| 6 | 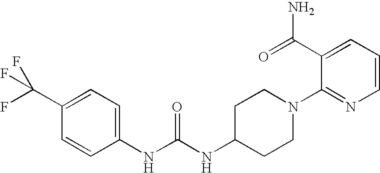 | 2-(4-(3-(4-(trifluoro- methyl)phenyl)ureido)- piperidin-1-yl)nicotinamide |
| 7 | 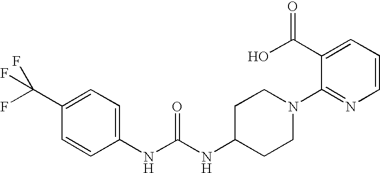 | 2-(4-(3-(4-trifluoro- methylphenyl)ureido)- piperidin-1-yl)nicotinic acid |
| 8 | 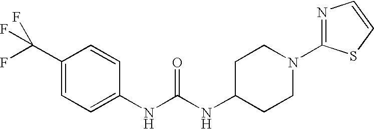 | 1-(1-(thiazol-2-yl)piperidin-4- yl)-3-(4- (trifluoromethyl)phenyl)urea |
| 9 | 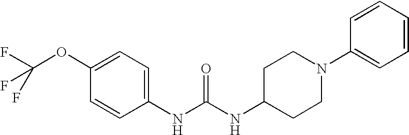 | 1-(1-phenylpiperidin-4-yl)-3- (4- (trifluoromethoxy)phenyl)urea |
| 10 | 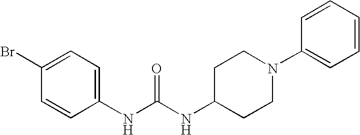 | 1-(4-bromophenyl)-3-(1- phenylpiperidin-4-yl)urea |
| 11 | 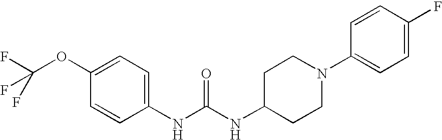 | 1-(1-(4- fluorophenyl)piperidin-4-yl)- 3-(4- (trifluoromethoxy)phenyl)urea |
| 12 | 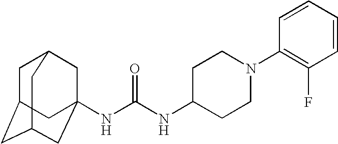 | 1-adamantyl-3-(1-(2- fluorophenyl)piperidin-4- yl)urea |
| 13 | 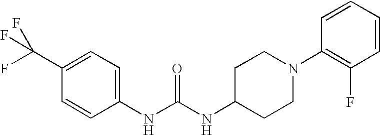 | 1-(1-(2- fluorophenyl)piperidin-4-yl)- 3-(4- (trifluoromethyl)phenyl)urea |

TABLE 8

| Compound | | |
|---|---|---|
| No. | Structure | Name |
| 1 | 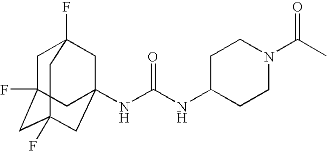 | 1-(1-acetylpiperidin-4-yl)-3- (3,5,7-trifluoroadamant-1- yl)urea |
| 2 | 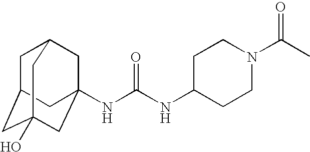 | 1-(1-acetylpiperidin-4-yl)-3-(3- hydroxyadamant-1-yl)urea |
| 3 | 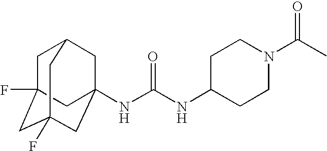 | 1-(1-acetylpiperidin-4-yl)-3- (3,5-difluoro adamant-1-yl)urea |
| 4 | 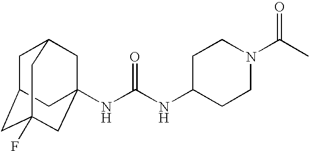 | 1-(1-acetylpiperidin-4-yl)-3-(3- fluoroadamant-1-yl)urea |
| 5 | 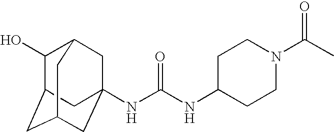 | 1-(1-acetylpiperidin-4-yl)-3-(4- hydroxyadamant-1-yl)urea |
| 6 | 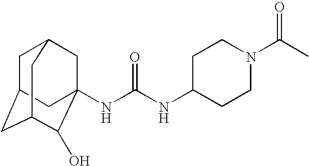 | 1-(1-acetylpiperidin-4-yl)-3-(2- hydroxyadamant-1-yl)urea |
| 7 | 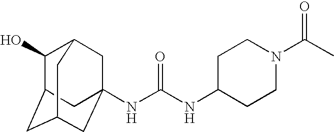 | (R)-1-(1-acetylpiperidin-4-yl)- 3-(4-hydroxyadamant-1- yl)urea |
| 8 | 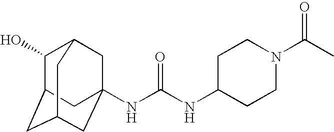 | (S)-1-(1-acetylpiperidin-4-yl)- 3-(4-hydroxyadamant-1- yl)urea |
| 9 | 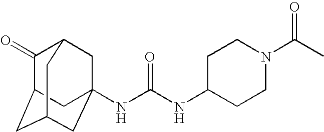 | 1-(1-acetylpiperidin-4-yl)-3-(4- oxoadamantyl)urea |
| 10 | 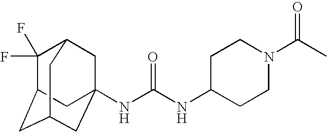 | 1-(1-acetylpiperidin-4-yl)-3- (4,4-difluoroadamantyl)urea |
| 11 | 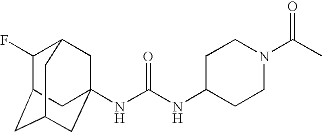 | 1-(1-acetylpiperidin-4-yl)-3-(4- fluoroadamantyl)urea |

TABLE 9

| Compound | | |
|---|---|---|
| No. | Structure | Name |
| 1 | 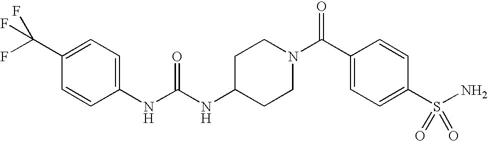 | 4-(4-(3-(4- (trifluoromethyl)phenyl)ureido)- piperidine-1- carbonyl)benzene-sulfonamide |
| 2 | 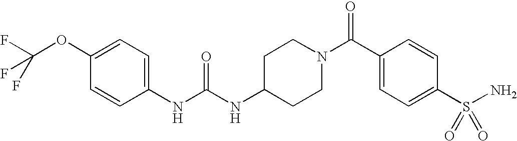 | 4-(4-(3-(4-(trifluoromethoxy)- phenyl)ureido)-piperidine-1- carbonyl)benzenesulfonamide |
| 3 | 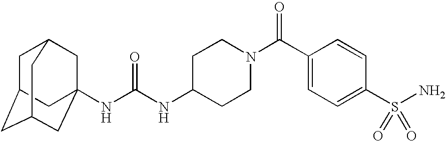 | 4-(4-(3-(1-adamantyl)ureido)- piperidine-1- carbonyl)benzene-sulfonamide |
| 4 | 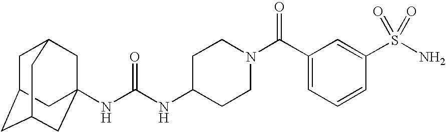 | 3-(4-(3-(1-adamantyl)ureido)- piperidine-1- carbonyl)benzene-sulfonamide |
| 5 | 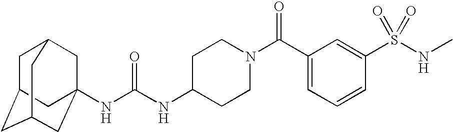 | 3-(4-(3-(1-adamantyl)ureido)- piperidine-1-carbonyl)-N- methylbenzene-sulfonamide |
| 6 | 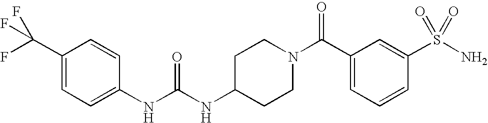 | 3-(4-(3-(4- (trifluoromethyl)phenyl)ureido)- piperidine-1- carbonyl)benzene-sulfonamide |
| 7 | 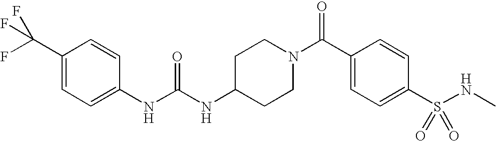 | 4-(4-(3-(4- (trifluoromethyl)phenyl)ureido)- piperidine-1-carbonyl)-N- methylbenzene-sulfonamide |
| 8 | 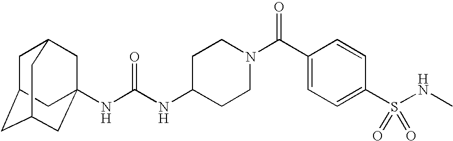 | 4-(4-(3-(1-adamantyl)ureido)- piperidine-1-carbonyl)-N- methylbenzene-sulfonamide |
| 9 | 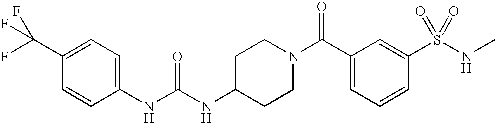 | N-methyl-3-(4-(3-(4- (trifluoromethyl)phenyl)ureido)- piperidine-1- carbonyl)benzene-sulfonamide |
| 10 | 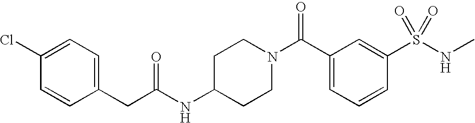 | 2-(4-chlorophenyl)-N-(1-(3- (N-methyl- sulfamoyl)benzoyl)-piperidin- 4-yl)acetamide |
| 11 | 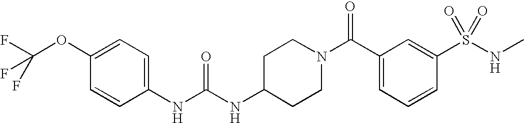 | N-methyl-3-(4-(3-(4- (trifluoromethoxy)- phenyl)ureido)-piperidine-1- carbonyl)benzene-sulfonamide |
| 12 | 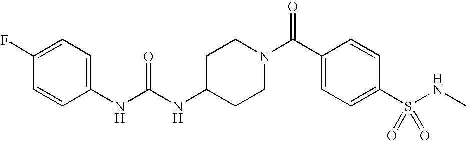 | 4-(4-(3-(4- fluorophenyl)ureido)piperidine- 1-carbonyl)-N- methylbenzene-sulfonamide |

TABLE 10

| Compound | | |
|---|---|---|
| No. | Structure | Name |
| 1 | 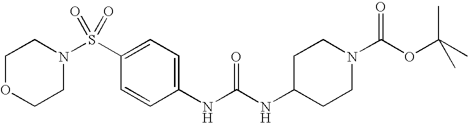 | tert-butyl 4-(3-(4- (morpholinosulfonyl)- phenyl)ureido)-piperidine-1- carboxylate |
| 2 | 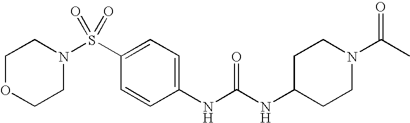 | 1-(1-acetylpiperidin-4-yl)-3-(4- (morpholinosulfonyl)phenyl)urea |

TABLE 11

| Compound | | |
|---|---|---|
| No. | Structure | Name |
| 1 | 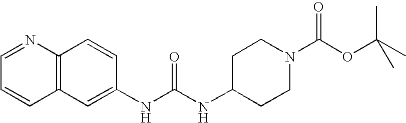 | tert-butyl 4-(3-quinolin-6-yl- ureido)piperidine-1- carboxylate |
| 2 | 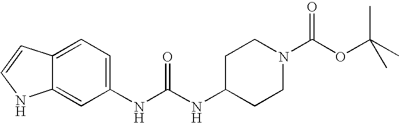 | tert-butyl 4-(3-1H-indol-6-yl- ureido)piperidine-1- carboxylate |
| 3 | 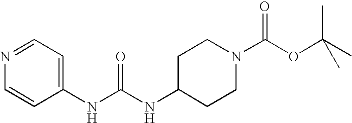 | tert-butyl 4-(3-pyridin-4-yl- ureido)piperidine-1- carboxylate |
| 4 | 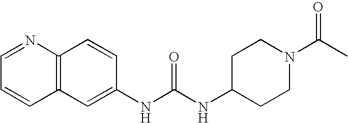 | 1-(1-acetylpiperidin-4-yl)-3- (quinolin-6-yl)urea |
| 5 | 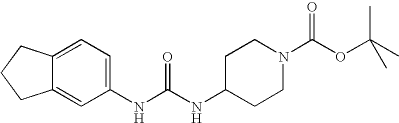 | tert-butyl 4-(3-(2,3-dihydro- 1H-inden-5-yl)ureido)- piperidine-1-carboxylate |
| 6 | 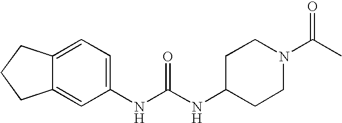 | 1-(1-acetyl-piperidin-4-yl)-3- (2,3-dihydro-1H-inden-5- yl)urea |
| 7 | 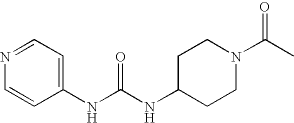 | 1-(1-acetyl-piperidin-4-yl)-3- (pyridin-4-yl)urea |
| 8 | 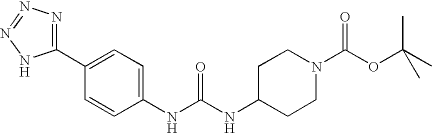 | tert-butyl 4-(3-(4-(1H- tetrazol-5-yl)phenyl)- ureido)piperidine-1- carboxylate |
| 9 | 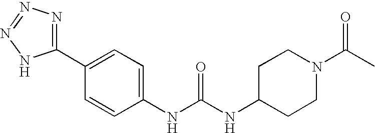 | 1-(4-(1H-tetrazol-5- yl)phenyl)-3-(1- acetylpiperidin-4-yl)urea |
| 10 | 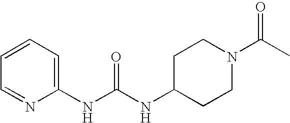 | 1-(1-acetylpiperidin-4-yl)-3- (pyridin-2-yl)urea |
| 11 | 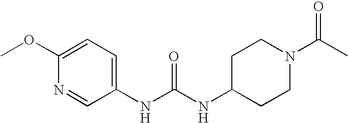 | 1-(1-acetylpiperidin-4-yl)-3- (6-methoxypyridin-3-yl)urea |
| 12 | 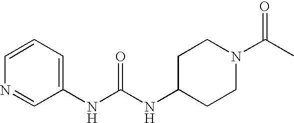 | 1-(1-acetylpiperidin-4-yl)-3- (pyridin-3-yl)urea |
| 13 | 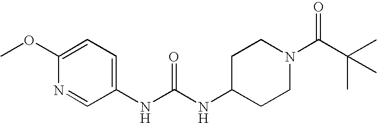 | 1-(6-methoxypyridin-3-yl)-3- (1-pivaloylpiperidin-4-yl)urea |
| 14 | 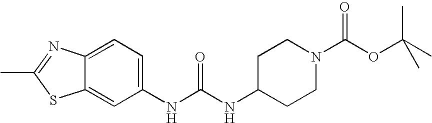 | tert-butyl 4-(3-(2- methylbenzo[d]thiazol-6- yl)ureido)piperidine-1- carboxylate |
| 15 | 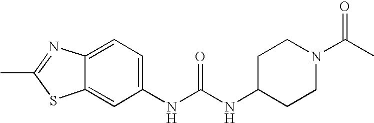 | 1-(1-acetylpiperidin-4-yl)-3- (2-methylbenzo[d]thiazol-6- yl)urea |
| 16 | 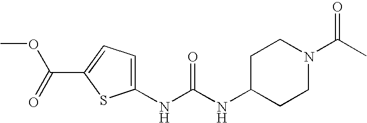 | methyl 5-(3-(1- acetylpiperidin-4- yl)ureido)thiophene-2- carboxylate |
| 17 | 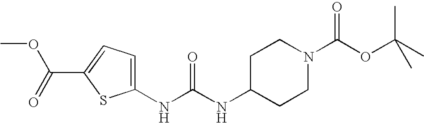 | tert-butyl 4-(3-(5- (methoxycarbonyl)thiophen- 2-yl)ureido)piperidine-1- carboxylate |
| 18 | 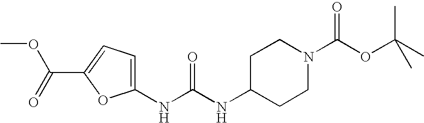 | tert-butyl 4-(3-(5- (methoxycarbonyl)furan-2- yl)ureido)piperidine-1- carboxylate |
| 19 | 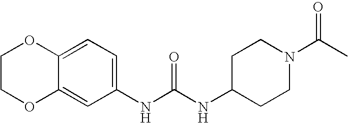 | 1-(1-acetylpiperidin-4-yl)-3- (2,3- dihydrobenzo[b][1,4]dioxin- 6-yl)urea |

TABLE 12

| Compound | | |
|---|---|---|
| No. | Structure | Name |
| 1 | 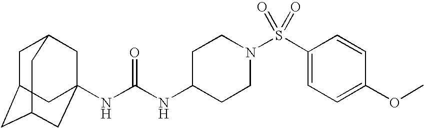 | 1-(1-adamantyl)-3-(1-(4- methoxyphenylsulfonyl)- piperidin-4-yl)urea |
| 2 | 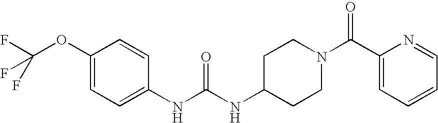 | 1-(1-picolinoylpiperidin-4-yl)- 3-(4-(trifluoro- methoxy)phenyl)urea |
| 3 | 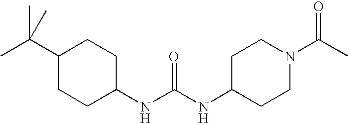 | 1-(1-acetylpiperidin-4-yl)-3- (4-tert-butyl-cyclohexyl)urea |
| 4 | 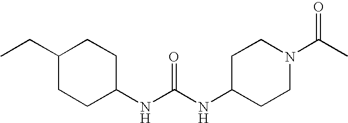 | 1-(1-acetylpiperidin-4-yl)-3- (4-ethylcyclohexyl)urea |
| 5 | 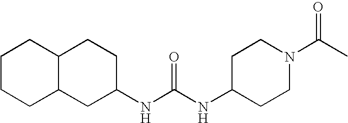 | 1-(1-acetylpiperidin-4-yl)-3- (decahydronaphthalen-2- yl)urea |
| 6 | 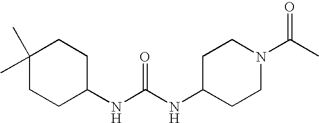 | 1-(1-acetylpiperidin-4-yl)-3- (4,4-dimethyl-cyclohexyl)urea |
| 7 | 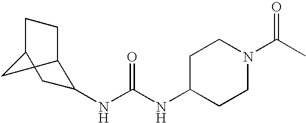 | 1-(1-acetylpiperidin-4-yl)-3- (bicyclo[2.2.1]heptan-2- yl)urea |
| 8 | 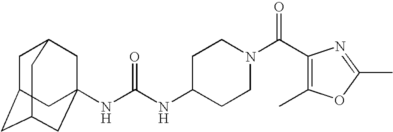 | 1-(1-adamantyl)-3-(1-(2,5- dimethyloxazole-4- carbonyl)piperidin-4-yl)urea |
| 9 | 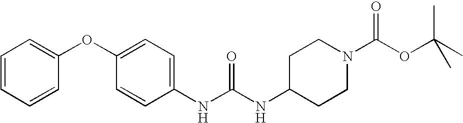 | tert-butyl 4-(3-(4- phenoxyphenyl)ureido)- piperidine-1-carboxylate |
| 10 | 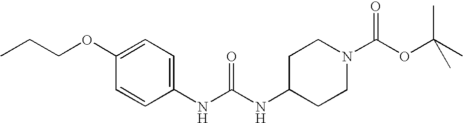 | tert-butyl 4-(3-(4- propoxyphenyl)ureido)- piperidine-1-carboxylate |
| 11 | 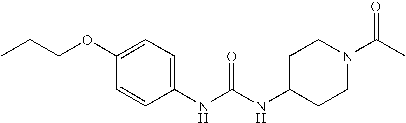 | 1-(1-acetylpiperidin-4-yl)-3- (4-propoxyphenyl)urea |
| 12 | 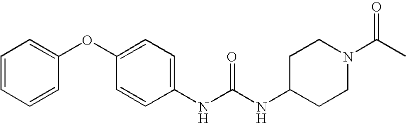 | 1-(1-acetylpiperidin-4-yl)-3- (4-phenoxyphenyl)urea |
| 13 | 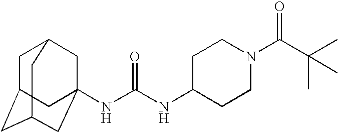 | 1-(1-adamantyl)-3-(1- pivaloylpiperidin-4-yl)urea |
| 14 | 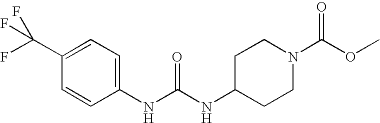 | methyl 4-(3-(4- (trifluoromethyl)phenyl)ureido)- piperidine-1-carboxylate |
| 15 | 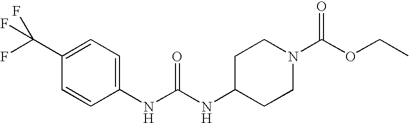 | ethyl 4-(3-(4- (trifluoromethyl)phenyl)ureido)- piperidine-1-carboxylate |
| 16 | 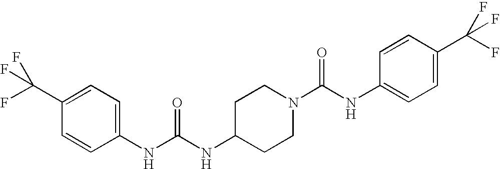 | N-(4-(trifluoromethyl)phenyl)- 4-(3-(4-(trifluoro- methyl)phenyl)ureido)- piperidine-1-carboxamide |
| 17 | 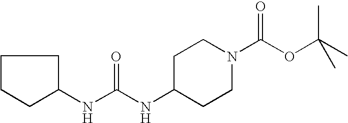 | tert-butyl 4-(3- cyclopentylureido)-piperidine- 1-carboxylate |
| 18 | 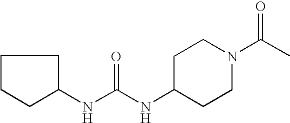 | 1-(1-acetylpiperidin-4-yl)-3- cyclopentylurea |
| 19 | 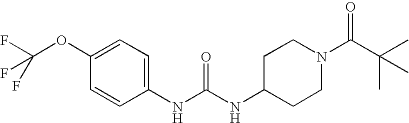 | 1-(1-pivaloylpiperidin-4-yl)-3- (4-(trifluoro- methoxy)phenyl)urea |
| 20 | 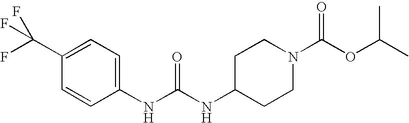 | isopropyl 4-(3-(4- (trifluoromethyl)phenyl)ureido)- piperidine-1-carboxylate |
| 21 | 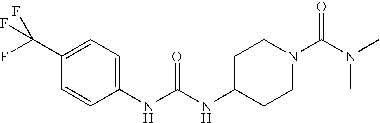 | N,N-dimethyl-4-(3-(4- (trifluoromethyl)phenyl)- ureido)piperidine-1- carboxamide |
| 22 | 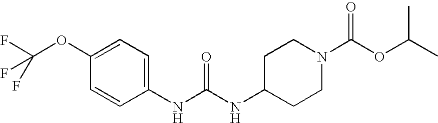 | isopropyl 4-(3-(4- (trifluoromethoxy)phenyl)ureido)- piperidine-1-carboxylate |
| 23 | 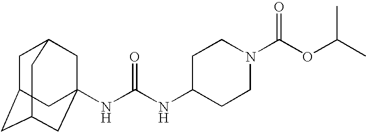 | isopropyl 4-(3-(1- adamantyl)ureido)-piperidine- 1-carboxylate |
| 24 | 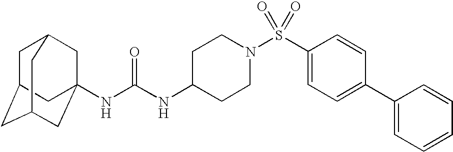 | 1-(1-(biphenyl-4- ylsulfonyl)piperidin-4-yl)-3- adamantylurea |
| 25 | 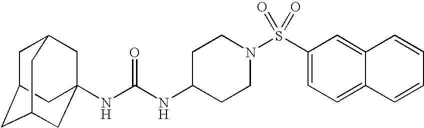 | 1-adamantyl-3-(1-(naphthalen- 262-ylsulfonyl)piperidin-4- yl)urea |
| 26 | 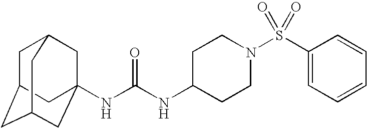 | 1-adamantyl-3-(1- (phenylsulfonyl)piperidin-4- yl)urea |
| 27 | 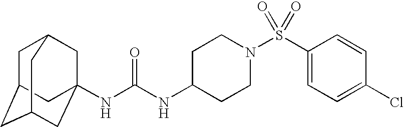 | 1-(1-(4- chlorophenylsulfonyl)piperidin- 4-yl)-3-cyclohexylurea |
| 28 | 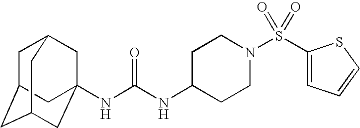 | 1-adamantyl-3-(1-(thiophen-2- ylsulfonyl)piperidin-4-yl)urea |
| 29 | 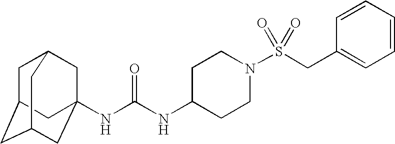 | 1-(1- (benzylsulfonyl)piperidin-4- yl)-3-adamantylurea |
| 30 | 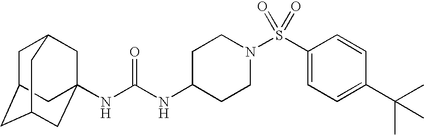 | 1-(1-(4-tert- butylphenylsulfonyl)piperidin- 4-yl)-3-adamantylurea |
| 31 | 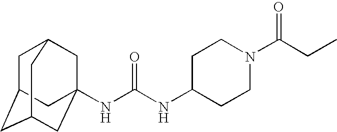 | 1-cyclohexyl-3-(1- propionylpiperidin-4-yl)urea |
| 32 | 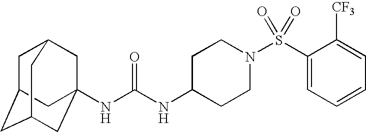 | 1-adamantyl-3-(1-(2- (trifluoromethyl)phenylsulfonyl)- piperidin-4-yl)urea |
| 33 | 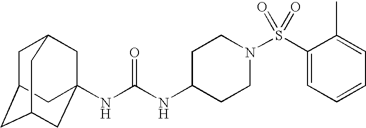 | 1-adamantyl-3-(1-(o- tolylsulfonyl)piperidin-4- yl)urea |
| 34 | 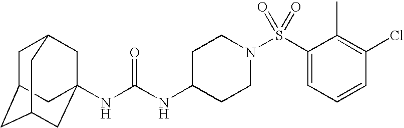 | 1-(1-(3-chloro-2- methylphenylsulfonyl)piperidin- 4-yl)-3-adamantylurea |
| 35 | 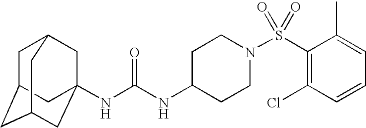 | 1-(1-(2-chloro-6- methylphenylsulfonyl)piperidin- 4-yl)-3-adamantylurea |
| 36 | 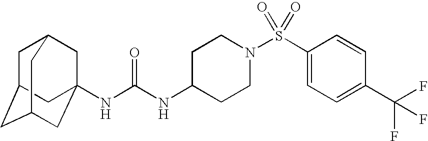 | 1-adamantyl-3-(1-(4- (trifluoromethyl)phenylsulfonyl)- piperidin-4-yl)urea |
| 37 | 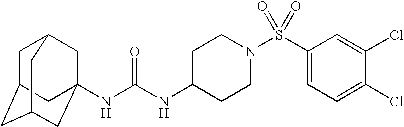 | 1-cyclohexyl-3-(1-(3,4- dichlorophenylsulfonyl)piperidin- 4-yl)urea |
| 38 | 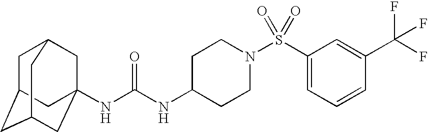 | 1-adamantyl-3-(1-(3- (trifluoromethyl)phenylsulfonyl)- piperidin-4-yl)urea |
| 39 | 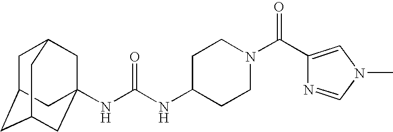 | 1-adamantyl-3-(1-(1-methyl- 1H-imidazole-4- carbonyl)piperidin-4-yl)urea |
| 40 | 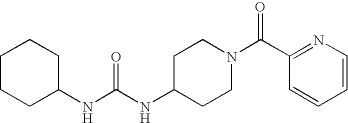 | 1-cyclohexyl-3-(1- picolinoylpiperidin-4-yl)urea |
| 41 | 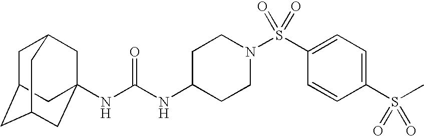 | 1-adamantyl-3-(1-(4- (methylsulfonyl)phenylsulfonyl)- piperidin-4-yl)urea |
| 42 | 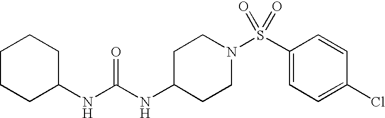 | 1-(1-(4- chlorophenylsulfonyl)piperidin- 4-yl)-3-cyclohexylurea |
| 43 | 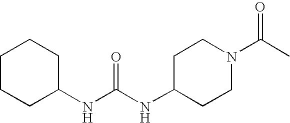 | 1-(1-acetylpiperidin-4-yl)-3- cyclohexylurea |
| 44 | 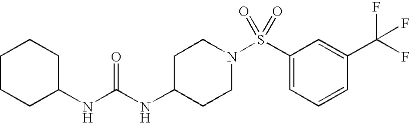 | 1-cyclohexyl-3-(1-(3- (trifluoromethyl)phenylsulfonyl)- piperidin-4-yl)urea |
| 45 | 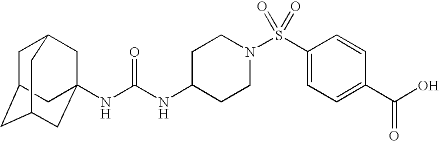 | 4-(4-(3- adamantylureido)piperidin-1- ylsulfonyl)benzoic acid |
| 46 | 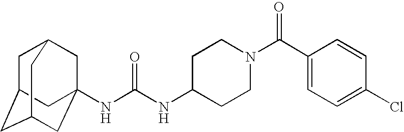 | 1-(1-(4- chlorobenzoyl)piperidin-4-yl)- 3-adamantylurea |
| 47 | 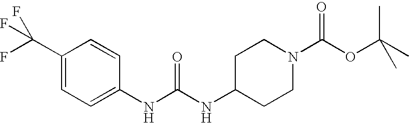 | tert-butyl 4-(3-(4- (trifluoromethyl)phenyl)ureido)- piperidine-1-carboxylate |
| 48 | 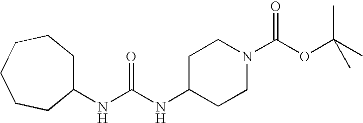 | tert-butyl 4-(3- cycloheptylureido)piperidine- 1-carboxylate |
| 49 | 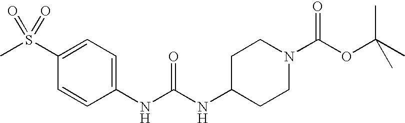 | tert-butyl 4-(3-(4- (methylsulfonyl)phenyl)ureido)- piperidine-1-carboxylate |
| 50 | 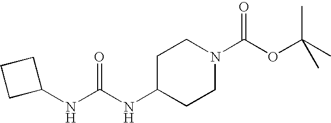 | tert-butyl 4-(3- cyclobutylureido)piperidine-1- carboxylate |
| 51 | 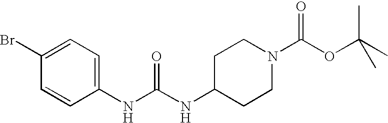 | tert-butyl 4-(3-(4- bromophenyl)ureido)piperidine- 1-carboxylate |
| 52 | 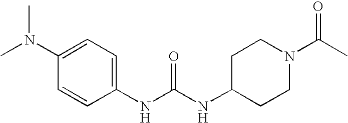 | 1-(1-acetylpiperidin-4-yl)-3- (4- (dimethylamino)phenyl)urea |
| 53 | 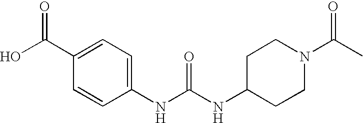 | 4-(3-(1-acetylpiperidin-4- yl)ureido)benzoic acid |
| 54 | 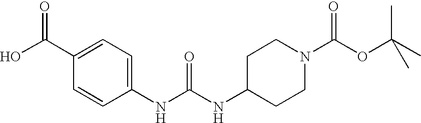 | 4-(3-(1-(tert- butoxycarbonyl)piperidin-4- yl)ureido)benzoic acid |
| 55 | 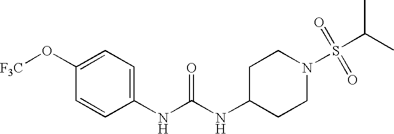 | 1-(1- (isopropylsulfonyl)piperidin-4- yl)-3-(4- (trifluoromethoxy)phenyl)urea |
| 56 | 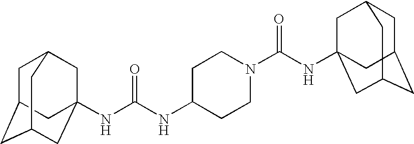 | N-adamantyl-4-(3- adamantylureido)piperidine-1- carboxamide |
| 57 | 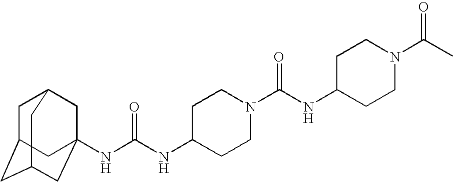 | N-(1-acetylpiperidin-4-yl)-4- (3- adamantylureido)piperidine-1- carboxamide |
| 58 | 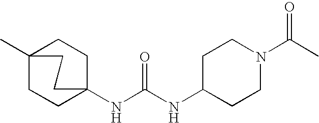 | 1-(1-acetylpiperidin-4-yl)-3- (4-methylbicyclo[2.2.2]octan- 1-yl)urea |
| 59 | 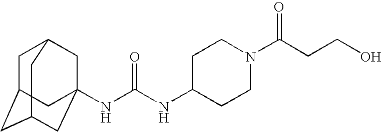 | 1-adamantyl-3-(1-(3- hydroxypropanoyl)piperidin-4- yl)urea |
| 60 | 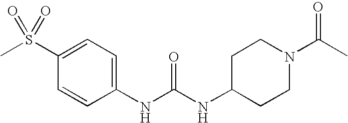 | 1-(1-acetylpiperidin-4-yl)-3- (4- (methylsulfonyl)phenyl)urea |
| 61 | 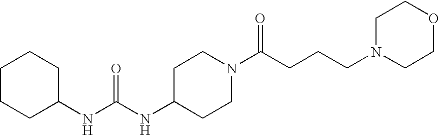 | 1-cyclohexyl-3-(1-(4- morpholinobutanoyl)piperidin- 4-yl)urea |
| 62 | 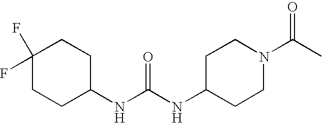 | 1-(1-acetylpiperidin-4-yl)-3- (4,4-difluorocyclohexyl)urea |
| 63 | 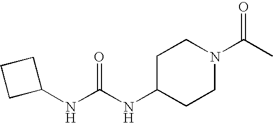 | 1-(1-acetylpiperidin-4-yl)-3- cyclobutylurea |
| 64 | 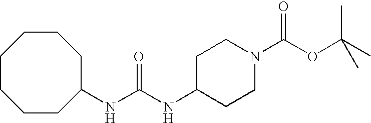 | tert-butyl 4-(3- cyclooctylureido)piperidine-1- carboxylate |
| 65 | 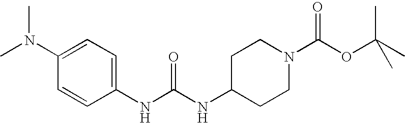 | tert-butyl 4-(3-(4- (dimethylamino)phenyl)ureido)- piperidine-1-carboxylate |
| 66 | 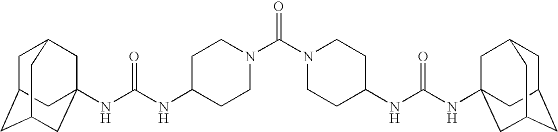 | 1,1′-(1,1′- carbonylbis(piperidine-4,1- diyl))bis(3-adamantylurea) |
| 67 | 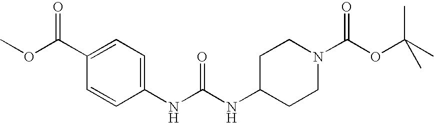 | tert-butyl 4-(3-(4- (methoxycarbonyl)phenyl)ureido)- piperidine-1-carboxylate |
| 68 | 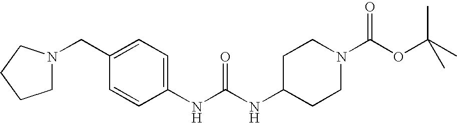 | tert-butyl 4-(3-(4-(pyrrolidin- 1- ylmethyl)phenyl)ureido)piperidine- 1-carboxylate |
| 69 | 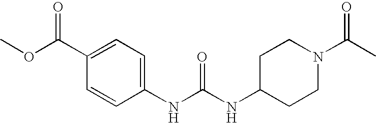 | methyl 4-(3-(1-acetylpiperidin- 4-yl)ureido)benzoate |
| 70 | 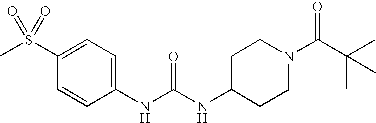 | 1-(4-(methylsulfonyl)phenyl)- 3-(1-pivaloylpiperidin-4- yl)urea |
| 71 | 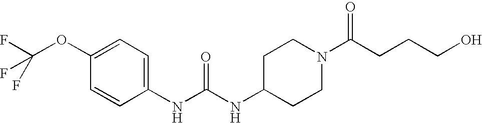 | 1-(1-(4- hydroxybutanoyl)piperidin-4- yl)-3-(4- (trifluoromethoxy)phenyl)urea |
| 72 | 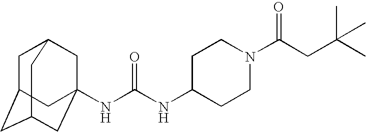 | 1-adamantyl-3-(1-(3,3- dimethylbutanoyl)piperidin-4- yl)urea |
| 73 | 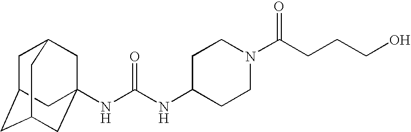 | 1-adamantyl-3-(1-(4- hydroxybutanoyl)piperidin-4- yl)urea |
| 74 | 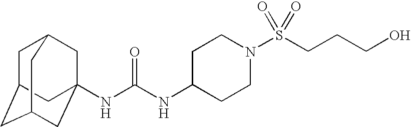 | 1-adamantyl-3-(1-(3- hydroxypropylsulfonyl)piperidin- 4-yl)urea |
| 75 | 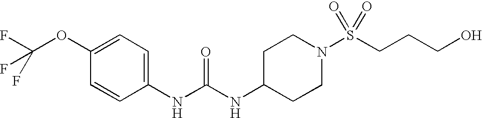 | 1-(1-(3- hydroxypropylsulfonyl)piperidin- 4-yl)-3-(4- (trifluoromethoxy)phenyl)urea |
| 76 | 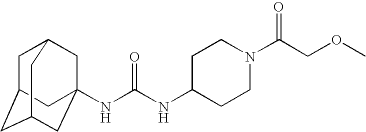 | 1-adamantyl-3-(1-(2- methoxyacetyl)piperidin-4- yl)urea |
| 77 | 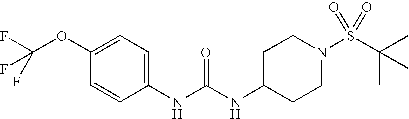 | 1-(1-tert- butylsulfonyl)piperidin-4-yl)- 3-(4- (trifluoromethoxy)phenyl)urea |
| 78 | 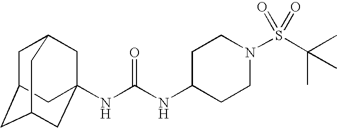 | 1-(1-(tert- butylsulfonyl)piperidin-4-yl)- 3-adamantylurea |
| 79 | 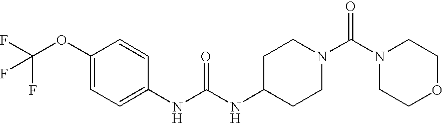 | 1-(1-(morpholine-4- carbonyl)piperidin-4-yl)-3-(4- (trifluoromethoxy)phenyl)urea |
| 80 | 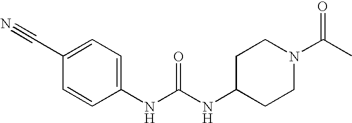 | 1-(1-acetylpiperidin-4-yl)-3- (4-cyanophenyl)urea |
| 81 | 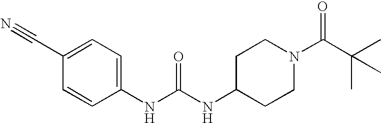 | 1-(4-cyanophenyl)-3-(1- pivaloylpiperidin-4-yl)urea |
| 82 | 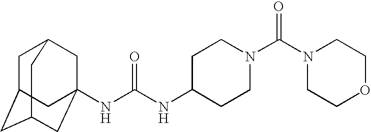 | 1-adamantyl-3-(1- (morpholine-4- carbonyl)piperidin-4-yl)urea |
| 83 | 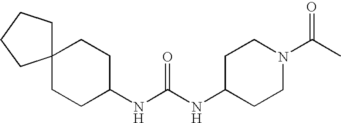 | 1-(1-acetylpiperidin-4-yl)-3- (spiro[4.5]decan-8-yl)urea |
| 84 | 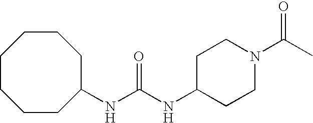 | 1-(1-acetylpiperidin-4-yl)-3- cyclooctylurea |
| 85 | 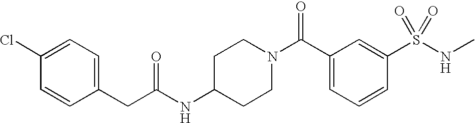 | 2-(4-chlorophenyl)-N-(1-(3- (N-methyl- sulfamoyl)benzoyl)-piperidin- 4-yl)acetamide |
| 86 | 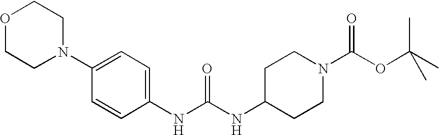 | tert-butyl 4-(3-(4- morpholinophenyl)ureido)piperidine- 1-carboxylate |
| 87 | 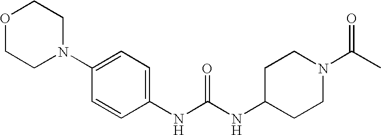 | 1-(1-acetylpiperidin-4-yl)-3- (4-morpholinophenyl)urea |
| 88 | 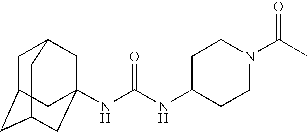 | 1-(1-acetylpiperidin-4-yl)-3- (adamantyl)urea |
| 89 | 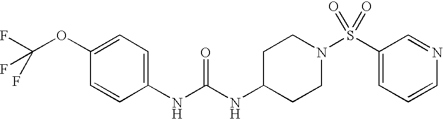 | 1-(1-(pyridin-3- ylsulfonyl)piperidin-4-yl)-3- (4- (trifluoromethoxy)phenyl)urea |
| 90 | 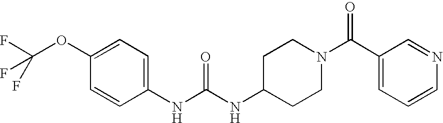 | 1-(1-nicotinoylpiperidin-4-yl)- 3-(4- (trifluoromethoxy)phenyl)urea |
| 91 | 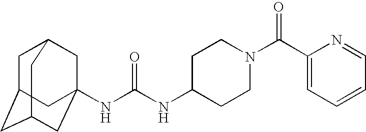 | 1-(adamantyl)-3-(1- picolinoylpiperidin-4-yl)urea |
| 92 | 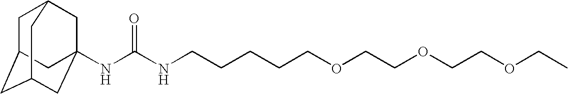 | 1-adamantyl-3-(5-(2-(2- ethoxyethoxy)ethoxy)pentyl)urea |
| 93 | 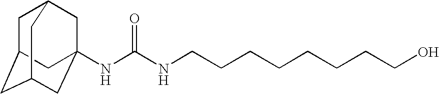 | 1-adamantyl-3-(8- hydroxyoctyl)urea |
| 94 | 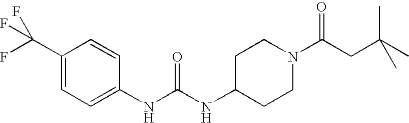 | 1-(1-(3,3- dimethylbutanoyl)piperidin-4- yl)-3-(4- (trifluoromethyl)phenyl)urea |
| 95 | 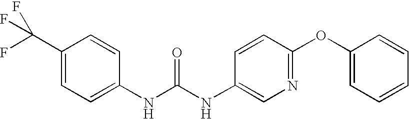 | 1-(6-phenoxypyridin-3-yl)-3- (4- (trifluoromethyl)phenyl)urea |
| 96 | 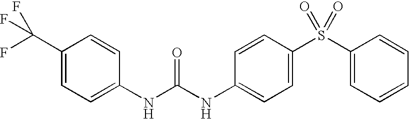 | 1-(4-(phenylsulfonyl)phenyl)- 3-(4- (trifluoromethyl)phenyl)urea |
| 97 | 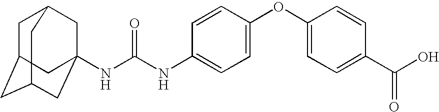 | 4-(4-(3- (adamantyl)ureido)phenoxy)- benzoic acid |
| 98 | 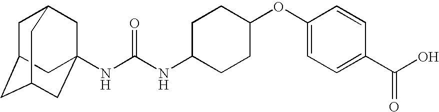 | 4-(4-(3- (adamantyl)ureido)cyclohexyl- oxy)benzoic acid |
| 99 | 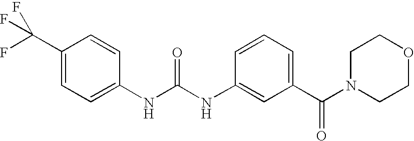 | 1-(3-(morpholine-4- carbonyl)phenyl)-3-(4- (trifluoromethyl)phenyl)urea |

### 3. Compositions and Formulations

The compositions used in the methods of the invention are comprised of, in general, a one or more compound(s) of Formula I-VII along with at least one pharmaceutically acceptable carrier or excipient. Acceptable carriers are known in the art and described supra. Acceptable excipients are non-toxic, aid administration, and do not adversely affect the therapeutic benefit of the compound. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols.

For more generalized therapeutic purposes, wherein the inflammatory condition is present with a syndrome of accompanying illnesses, combination therapy is often desirable. Combination therapy includes administration of a single pharmaceutical dosage formulation which contains one or more compound(s) of Formula I-VII and one or more additional active agents, or therapies such as heat, light and such, as well as administration of one or more compound(s) of Formula I-VII and each active agent in its own separate pharmaceutical dosage formulation. For example, one or more compound(s) of Formula I-VII and one or more of other agents such as non-steroidal anti-inflammatory agents (NSAIDs), corticosteroids and disease-modifying antirheumatic drugs (DMARDs), and the like; can be administered to the human subject together in a single oral dosage composition, such as a tablet or capsule, or each agent can be administered in separate oral dosage formulations. Where separate dosage formulations are used, the one or more compound(s) of Formula I-VII and one or more additional active agents can be administered at essentially the same time (i.e., concurrently), or at separately staggered times (i.e., sequentially). Combination therapy is understood to include all these regimens.

Complimentary and synergistic compounds can be selected by ability to increase the levels of cis-Epoxyeicosantrienoic acids (“EETs”). This permits EETs to be used in conjunction with one or more compounds of Formula I-VII to reduce inflammation in the methods of the invention. It further permits EETs to be used in conjunction with one or more compounds of Formula I-VII to reduce clinical and sub-clinical effects described herein. Thus, medicaments of EETs can be made which can be administered in conjunction with one or more compounds of Formula I-VII, or a medicament containing one or more compounds of Formula I-VII can optionally contain one or more EETs.

EETs, which are epoxides of arachidonic acid, are known to be effectors of blood pressure, regulators of inflammation, and modulators of vascular permeability. Hydrolysis of the epoxides by sEH diminishes this activity. Inhibition of sEH raises the level of EETs since the rate at which the EETs are hydrolyzed into DHETs is reduced. Without wishing to be bound by theory, it is believed that raising the level of EETs interferes with damage to kidney cells by the microvasculature changes and other pathologic effects of diabetic hyperglycemia. Therefore, raising the EET level in the kidney is believed to protect the kidney from progression from microalbuminuria to end stage renal disease.

EETs are well known in the art. EETs useful in the methods of the present invention include 14,15-EET, 8,9-EET and 11,12-EET, and 5,6 EETs, in that order of preference. Preferably, the EETs are administered as the methyl ester, which is more stable. Persons of skill will recognize that the EETs are regioisomers, such as 8S,9R- and 14R,15S-EET. 8,9-EET, 11,12-EET, and 14R,15S-EET, are commercially available from, for example, Sigma-Aldrich (catalog nos. E5516, E5641, and E5766, respectively, Sigma-Aldrich Corp., St. Louis, Mo.).

EETs produced by the endothelium have anti-hypertensive properties and the EETs 11,12-EET and 14,15-EET may be endothelium-derived hyperpolarizing factors (EDHFs). Additionally, EETs such as 11,12-EET have profibrinolytic effects, anti-inflammatory actions and inhibit smooth muscle cell proliferation and migration. In the context of the present invention, these favorable properties are believed to protect the vasculature and organs during renal and cardiovascular disease states.

It is understood that, like all drugs, inhibitors have half lives defined by the rate at which they are metabolized by or excreted from the body, and that the inhibitor will have a period following administration during which it will be present in amounts sufficient to be effective. If EETs are administered after the inhibitor is administered, then it is desirable that the EETs be administered during the period in which the inhibitor will be present in amounts to be effective to delay hydrolysis of the EETs. Typically, the EET or EETs will be administered within 48 hours of administering one or more compound(s) of Formula I-VII. Preferably, the EET or EETs are administered within 24 hours of administering a combination of one or more compound(s) of Formula I-VII, and even more preferably within 12 hours. In increasing order of desirability, the EET or EETs are administered within 10, 8, 6, 4, 2, hours, 1 hour, or one half hour after administration of a combination of one or more compound(s) of Formula I-VII. Most preferably, the EET or EETs are administered concurrently with the composition of the invention.

In certain embodiments, the EETs, the one or more compound(s) of Formula I-VII, or both, are provided in a material that permits them to be released over time to provide a longer duration of action. Slow release coatings are well known in the pharmaceutical art; the choice of the particular slow release coating is not critical to the practice of the present invention.

EETs are subject to degradation under acidic conditions. Thus, if the EETs are to be administered orally, it is desirable that they are protected from degradation in the stomach. Conveniently, EETs for oral administration may be coated to permit them to passage through the acidic environment of the stomach into the basic environment of the intestines. Such coatings are well known in the art. For example, aspirin coated with so-called “enteric coatings” is widely available commercially. Such enteric coatings may be used to protect EETs during passage through the stomach. An exemplary coating is set forth in the Examples.

Compressed gases may be used to disperse a composition of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The following are representative pharmaceutical formulations containing a compound of the present invention.

### Tablet Formulation

The following ingredients are mixed intimately and pressed into single scored tablets.

| Ingredient | Quantity per tablet, mg |
|---|---|
| Compound of the invention | 400 |
| Cornstarch | 50 |
| Croscarmellose sodium | 25 |
| Lactose | 120 |
| Magnesium stearate | 5 |

### Capsule Formulation

The following ingredients are mixed intimately and loaded into a hard-shell gelatin capsule.

| Ingredient | Quantity per capsule, mg |
|---|---|
| Compound of the invention | 200 |
| Lactose, spray-dried | 148 |
| Magnesium stearate | 2 |

### Suspension Formulation

The following ingredients are mixed to form a suspension for oral administration (q.s.=sufficient amount).

| Ingredient | Amount | |
|---|---|---|
| Compound of the invention | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.0 | g |
| Sorbitol (70% solution) | 13.0 | g |
| Veegum K (Vanderbilt Co) | 1.0 | g |
| Flavoring | 0.035 | mL |
| colorings | 0.5 | mg |
| distilled water | q.s. to 100 | mL |

### Injectable Formulation

The following ingredients are mixed to form an injectable formulation.

| Ingredient | Quantity per injection, mg |
|---|---|
| Compound of the invention | 0.2 mg-20 mg |
| sodium acetate buffer solution, 0.4 M | 2.0 mL |
| HCl (1N) or NaOH (1N) | q.s. to suitable pH |
| water (distilled, sterile) | q.s. to 20 mL |

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Quantity per suppository, mg |
|---|---|
| Compound of the invention | 500 mg |
| Witepsol ® H-15 | balance |

### 4. Dosing and Administration

The present invention provides therapeutic methods involving administering to a subject in need thereof an effective amount of a one or more compound(s) of Formula I-VII. The dose, frequency, and timing of such administering will depend in large part on the selected therapeutic agent, the nature of the condition to be treated, the condition of the subject, including age, weight and presence of other conditions or disorders, the formulation of the therapeutic agent and the discretion of the attending physician. The compositions of the invention are administered via oral, parenteral, subcutaneous, intramuscular, intravenous or topical routes. Generally, the compounds are administered in dosages ranging from about 2 mg up to about 2000 mg per day, although variations will necessarily occur, depending, as noted above, on the target tissue, the subject, and the route of administration. Dosages are administered orally in the range of about 0.05 mg/kg to about 20 mg/kg, more alternatively in the range of about 0.05 mg/kg to about 0.2 mg/kg of body weight per day. The dosage for topical administration will necessarily depend on the size of the area being treated, the disorder to be treated and the individual being treated.

The following examples are provided to illustrate certain aspects of the present invention and to aid those of skill in the art in practicing the invention. These examples are in no way to be considered to limit the scope of the invention.

### 5. Experimental Examples

### Synthetic Chemistry

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and G. M. Wuts, Protecting Groups in Organic Synthesis, Third Edition, Wiley, New York, 1999, and references cited therein.

Furthermore, the compounds of this invention may contain one or more chiral centers. Accordingly, if desired, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wis., USA), Bachem (Torrance, Calif., USA), Emka-Chemce or Sigma (St. Louis, Mo., USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplementals (Elsevier Science Publishers, 1989), Organic Reactions, Volumes 1-40 (John Wiley and Sons, 1991), March's Advanced Organic Chemistry, (John Wiley and Sons, 4^{th} Edition), and Larock's Comprehensive Organic Transformations (VCH Publishers Inc., 1989).

The various starting materials, intermediates, and compounds of the invention may be isolated and purified where appropriate using conventional techniques such as precipitation, filtration, crystallization, evaporation, distillation, and chromatography. Characterization of these compounds may be performed using conventional methods such as by melting point, mass spectrum, nuclear magnetic resonance, and various other spectroscopic analyses.

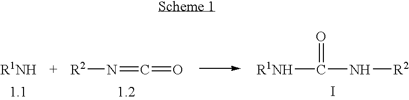

A synthesis of the compounds of the invention is shown in Scheme 1, where R¹ and R² are as defined herein. Amine 1.1 reacts with the appropriate isocyanate 1.2 to form the corresponding urea or thiourea of formula I. Typically, the formation of the urea is conducted using a polar solvent such as DMF (dimethylformamide) at 0 to 10° C. Isocyanate or thioisocyanate 1.2 can be either known compounds or compounds that can be prepared from known compounds by conventional synthetic procedures.

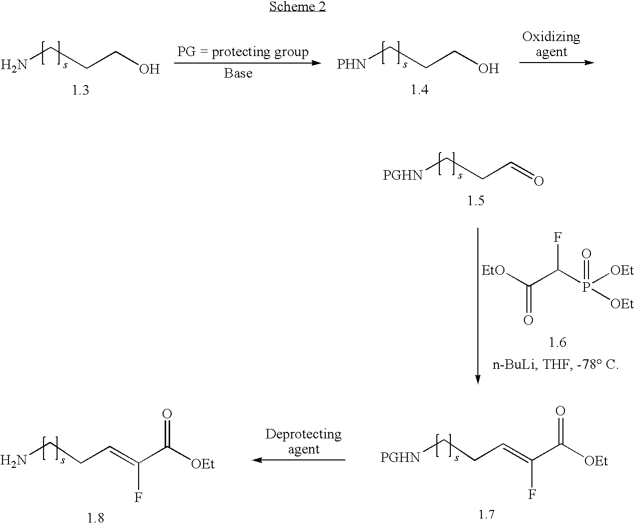

In Scheme 2, s is as defined herein. The synthesis of the compounds of the invention can be exemplified by, but is not limited to, the preparation of the intermediate 1.8, as shown in Scheme 2. Amine 1.3 can be protected with any amine protecting group known in the art (for example, 2,4-dimethoxy-benzyl (DMB), tert-butoxycarbonyl (Boc) etc.) to give compounds 1.4. For example, amine 1.3 can be treated with t-Boc anhydride in the presence of a base, such as sodium carbonate, and a suitable solvent such as, THF to give compounds 1.4. Upon reaction completion, 1.4 can be recovered by conventional techniques such as neutralization, extraction, precipitation, chromatography, filtration and the like; or, alternatively, used in the next step without purification and/or isolation.

Compounds 1.4 are then treated with any suitable oxidizing agent known in the art, to give aldehydes 1.5. For example, 1.4 can be treated with pyridinium chlorochromate (PCC) and neutral alumina (Al₂O₃) in the presence of a suitable solvent, such as, dichloromethane (DCM) to give 1.5. Upon reaction completion, 1.5 can be recovered by conventional techniques such as neutralization, extraction, precipitation, chromatography, filtration and the like; or, alternatively, used in the next step without purification and/or isolation.

Compounds 1.5 are then treated with triethyl-2-fluoro-2-phosphonoacetate 1.6 to give compounds 1.7. This is typically performed in dry tetrahydrofuran (THF) or another suitable solvent known to one skilled in the art, typically at, but not limited to, room temperature in the presence of n-butyllithium (n-BuLi), or another suitable base known to one skilled in the art. Upon reaction completion, 1.7 can be recovered by conventional techniques such as neutralization, extraction, precipitation, chromatography, filtration and the like; or, alternatively, used in the next step without purification and/or isolation.

Compounds 1.7 are then deprotected using a suitable deprotecting agent known in the art to give the intermediate 1.8. For example, deprotection can be achieved, in addition to other methods known to one skilled in the art, by treatment of 1.7 with SOCl₂ in a suitable solvent such as dichloromethane (DCM) (preferred method for PG=2,4-dimethoxy-benzyl (DMB)). Alternatively, 1.7 can be deprotected with TFA neat or in a suitable solvent known to one skilled in the art such as, DCM to give the compounds 1.8 (preferred method for PG=tert-butoxycarbonyl (Boc)). Upon reaction completion, 1.8 can be recovered by conventional techniques such as neutralization, extraction, precipitation, chromatography, filtration and the like; or, alternatively, used in the next step without purification and/or isolation.

The synthesis of the compounds of the invention can be exemplified by, but is not limited to, the use of the intermediate 1.8 to prepare the compounds of the invention, as shown in Scheme 3.

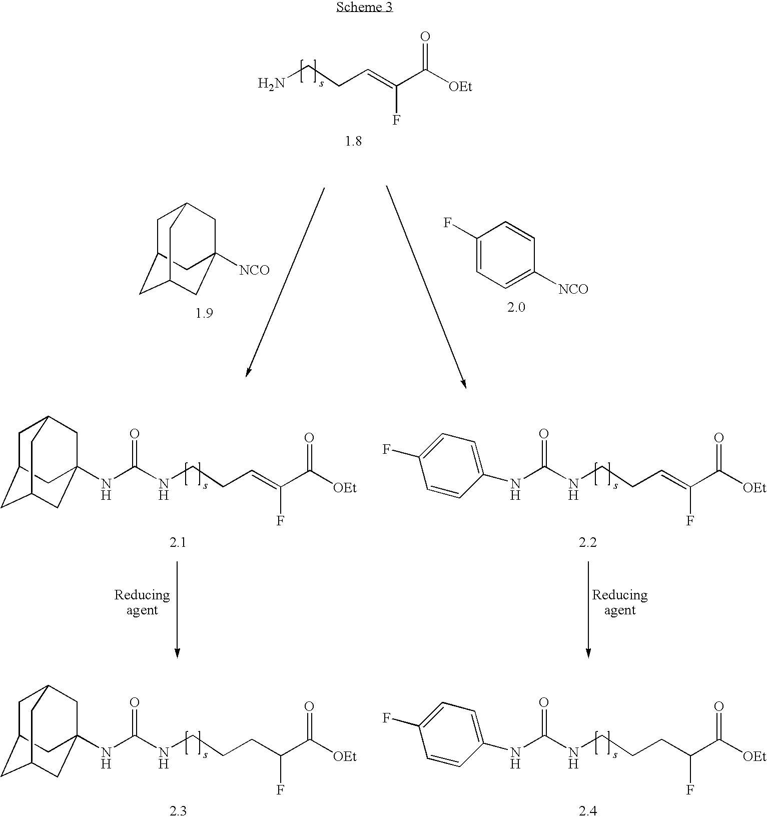

The intermediate 1.8 can be treated with appropriate isocyanate compounds 1.9 or 2.0 to form the corresponding adamantyl compounds 2.1 or phenyl compounds 2.2. Without limiting the scope of the present invention, Scheme 3 shows p-fluorophenyl or unsubstituted adamantyl for illustration purposes only. Any suitably substituted or unsubstituted phenyl or adamantyl can be used in Scheme 3 to yield the compounds of the invention. Typically, the reaction with isocyanates is conducted using DCM in the presence of triethylamine (TEA) at room temperature, or alternatively, a polar solvent such as DMF (dimethylformamide) at 0 to 10° C. Isocyanate compounds 1.9 or 2.0 can be either known compounds or compounds that can be prepared from known compounds by conventional synthetic procedures. Upon reaction completion, 2.1 and/or 2.2 can be recovered by conventional techniques such as neutralization, extraction, precipitation, chromatography, filtration and the like; or, alternatively, used in the next step without purification and/or isolation.

Compounds 2.1 or 2.2 can then be reduced using any suitable reducing agent known in the art, to give compounds 2.3 or 2.4, respectively. For example, 2.1 or 2.2 can be hydrogenated with palladium/carbon (Pd/C) in the presence of a suitable solvent known in the art such as, methanol, at suitable temperature such as, room temperature. Upon reaction completion, 2.3 and/or 2.4 can be recovered by conventional techniques such as neutralization, extraction, precipitation, chromatography, filtration and the like. Alternatively, the ester group of the adamantyl compounds 2.1 or phenyl compounds 2.2 can be hydrolyzed (not shown in Scheme 3) to give the corresponding acid compounds. The hydrolysis of esters is well known in the art. For example, the ester can be hydrolyzed using lithium hydroxide (LiOH) in the presence of a suitable solvent such as, but not limited to THF/methanol/water. The resulting acids can then be reduced with reducing agents as described above to give the corresponding adamantyl or phenyl compounds of the invention.

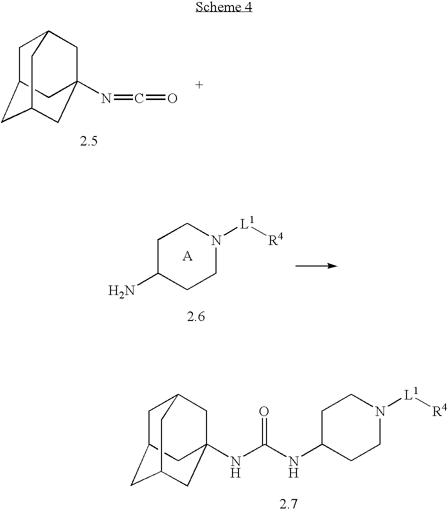

For example, compounds of formula II may be prepared as shown in Scheme 4 where ring A is a piperidinyl ring and L¹ and R⁴ are as defined herein. Reaction of isocyanate 2.5 with amine 2.6 forms the corresponding urea or thiourea of 2.7.

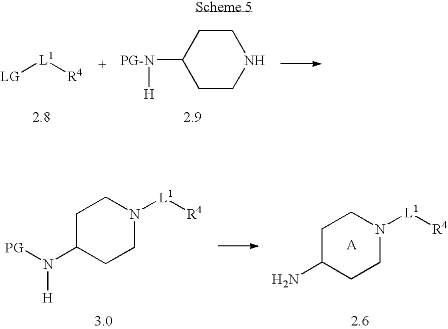

Amine 2.6 can be prepared according to Scheme 5, where LG represents a suitable leaving group such as a halide and PG is an amine protecting group such as a tert-butoxycarbonyl (Boc) group. Reaction of the appropriate 2.8 with protected aminopiperidine 2.9 forms the functionalized amine 3.0. Removal of the protecting group gives 2.6.

Preferred methods of making the compounds of the invention are described in US Application Publication No. 2008/0207908, which is incorporated herein by reference in its entirety.

The following examples are provided to illustrate certain aspects of the present invention and to aid those of skill in the art in practicing the invention. These examples are in no way to be considered to limit the scope of the invention.

### EXAMPLES

### Example 1

### Bioassays of TNF-α, IL-6 and Rheumatoid Factor

Release of both TNF-α and IL-6 into the bloodstream of animals is associated with events of inflammation. The inflammatory response in turn is associated with a number of predisposing conditions including but not limited to autoimmune and infectious disease. Bacterial endotoxin components of lipopolysaccharide (LPS) are imputed as the primary trigger in septic responses to infection by gram-negative bacteria. Animal models for testing responsiveness to therapeutic agents assay the release of cytokines in response to exposure to LPS. Levels of cytokines before and after treatment are indicators of the effectiveness of, for example, new antibiotic compounds (Prins et al., Infect. Immun. 63:2236-2242, (1995)).

To determine the level of effectiveness of treatment of subjects exhibiting inflammatory disease conditions, the methods of the invention employ standard assays for detection and assessment of released cytokines. By way of illustration only, methods for the blood or serum assay of TNF-α and IL-6 are described herein. Those of skill in the art will recognize that similar methods will apply to the assay procedures for other cytokines, thus, the following description is not intended to be limiting.

For assaying TNF-α, (Engelberts et al., Lymphokine Cytokine Res. 10:69-76, (1991)) anti-TNF-α antibody, directed to the 26 kd or 17 kd forms of TNF-α are immobilized and then incubated with the serum or blood sample containing unknown concentrations TNF-α (Engelberts et al., Lymphokine Cytokine Res. 10:69-76, (1991)). After allowing for a suitable period of incubation for antigen-antibody complexes to form, the immobilized antibody is incubated with an indicator solution containing a second anti-TNF-α antibody, either monoclonal or polyclonal, which has been labeled. This second anti-TNF-α antibody is allowed to incubate with immobilized antibody for a sufficient period of time to allow antigen-antibody complexes to form between the labeled antibody and any TNF-α bound by the immobilized monoclonal antibody. After this incubation, the immobilized monoclonal antibody is separated from any unbound labeled antibody, and the amount of label remaining bound to the immobilized antibody is measured. This can be done by measuring a calorimetric or spectrophotometric signal related to the amount of label present on the immobilized antibody. The signal, therefore, provides a measure of the amount of TNF-α in the fluid test sample.

Similarly, IL-6 is assayed most commonly by ELISA, such as provided in commercially available kits, such as the Quantikine IL-6 assay kit (R&D Systems, Minneapolis, Minn.). Briefly, a monoclonal antibody specific for IL-6 coated onto a microtiter plate is used to capture any IL-6 contained in each sample. After washing, an enzyme linked polyclonal antibody specific for IL-6 is added to allow detection of any bound IL-6. Optical density values of samples are recorded (using, for example, a microtiter plate reader from Hewlett Packard) and compared to those of an IL-6 standard curve (10-2000 pg/ml). In this method serum, plasma or whole blood sources of IL-6 binding to anti-IL-6 may be quantitated calorimetrically.

In clinical practice rheumatoid factor is determined by hemagglutination, latex agglutination, ELISA, the Waaler Rose or sheep-cell agglutination test and nephelometry with variants to these procedures continuing to be developed (Spiritus et al. Ann. Rheum. Dis. 63:1169-1171 (2004)). One variant on the Waaler Rose assay involves substituting gelatin particles (Serodia-RA, Fujirebio, Inc. Japan) for red blood cells to improve specificity of serum reactions. Another relatively recent assay combines detection of rheumatoid factor with detection of antibodies to cyclic citrullinated peptide (Kroot Arthritis Rheum. 43:1831-1835 (2000)).

The immunoassays utilized in the methods of the present invention are alternatively sandwich assays employing the antibodies disclosed herein, although other assay formats known in the art may also be used. Additionally, the immunoassays described herein may be adapted to a solid state resin that contains fluorescent tags which allow for quantitation of the amount of a cytokine with fluorescent particle sorting equipment such as that made by Luminex Technologies.

### Example 2

### s-EH Inhibitors can Reverse/Prevent Endothelial Dysfunction

(1) Angiotensin II (AngII) has been shown to increase the expression of s-EH in the vascular endothelium both in vitro and in vivo in rats (Ai et al., Proc. Natl. Acad. Sci. 104: 9019-9023, (2007)). The expression of s-EH was shown to be elevated after AngII treatment in human umbilical vein endothelial cells both at the RNA and the protein levels. Infusion of AngII into rats increased the s-EH levels in the aortic intima. In addition AngII causes low-grade vascular inflammation by inducing oxidative stress, resulting in NFkb up-regulation leading to endothelial dysfunction by reducing the amount of endothelial NO production/bioactivity (Savoia and Schiffrin, Clin. Sci. (Lond), 112: 375-84, (2007)). Theoretically, an s-EH inhibitor would reverse endothelial dysfunction, because (i) s-EH levels are elevated and (ii) elevated levels of EETs (as a consequence of s-EH inhibition) are effective inhibitors of NFkb up-regulation in endothelial cells (Node et al., Science 285:1276-79, (1999)) consequently of endothelial dysfunction under such conditions (e.g. hypertensive patients with Type II diabetes).

(2) EETs serve as endothelial hyperpolarizing factors (EDHF) and as such cause direct vasorelaxation. Under conditions of severe endothelial dysfunction, such as diabetes (Shi et al, J. Pharmacol. Exp. Ther., 318: 276-81, (2006); Pannirselvam et al. Eur. J. Pharmacol., 551: 98-107, (2006)), eNOS knockout mice (Huang et al. Am. J. Physiol., Heart Circ. Physiol., 280:H2462-9, (2001)) or eNOS inhibition (e.g. by L-NAME), EDHF (EETs) compensate to a certain degree for the loss of NO, and mediate endothelium dependent vasodilation triggered by agonists (e.g. bradykinin) or flow. Assuming that EETs levels are limited by s-EH activity in the endothelium, s-EH inhibitors should enhance EETs mediated endothelium-dependent vasodilation under such conditions, and thereby improve endothelial function.

(3) Direct proof that s-EH inhibition indeed improves endothelial function (endothelium-dependent vasodilation) has been provided by a recent paper of Lock et al (Lock et al. Cell Biochem. Biophys., 47:87-98, (2007)). In this study, the effect of the s-EH inhibitor, ADU (adamantyl dodecyl urea) was tested on endothelium-dependent relaxation by acetylcholine of thoracic aortic rings isolated from rats that either received sham surgery or were uninephrectomized followed by DOCA (deoxycorticosterone acetate) and salt treatment. The study involved four groups, normotensive rats receiving vehicle, normotensive rats receiving ADU, DOCA-salt hypertensive rats receiving vehicle and DOCA-salt hypertensive rats receiving ADU. Aortic rings obtained from hypertensive animals receiving DOCA-salt treatment were less responsive to acetylcholine (endothelial dysfunction). The thoracic aortic rings obtained from DOCA-salt treated animals that also received the s-EH inhibitor ADU, showed augmented acetylcholine-induced endothelium-dependent vasorelaxation, similar to that of the normotensive control animals.

The above results further demonstrate that the s-EH inhibitors described herein are capable of augmenting endothelial NO thereby improving endothelial function. These results lend themselves to a method for chronic augmentation of endothelial NO in a patient who is suffering from insufficient endothelial NO levels. Non-limiting examples of diseases associated with insufficient endothelial NO levels include diabetes, diabetes with hypertension, metabolic syndrome, obesity, insulin resistance without hypertension, insulin resistance with hypertension and dyslipidemia.

It is to be understood that while the invention has been described in conjunction with the above embodiments, that the foregoing description and examples are intended to illustrate and not limit the scope of the invention. Other aspects, advantages and modifications within the scope of the invention will be apparent to those skilled in the art to which the invention pertains.

What is claimed is:

1. A method for treating a disease or a symptom of a disease related to endothelial dysfunction in a subject, said method comprising administering to a subject in need of such treatment an effective amount of a compound of Formula I: 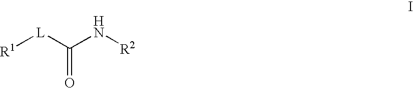
wherein
R¹ and R² independently are selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
L is —NH— or —CR′R″— where R′ and R″ are independently H or alkyl or R′ and R″ together form a C₃-C₆ cycloalkyl ring;
or a stereoisomer, a tautomer, or a pharmaceutically acceptable salt thereof, wherein the compound of Formula I is a soluble epoxide hydrolase inhibitor.

2. The method in accordance with claim 1, wherein R¹ is adamantyl or substituted adamantyl.

3. The method in accordance with claim 1, wherein L is —NH—.

4. The method in accordance with claim 1, wherein L is —CR′R″— where R′ and R″ are independently H or alkyl or R′ and R″ together form a C₃-C₆ cycloalkyl ring.

5. The method in accordance with claim 1, wherein R¹ is phenyl or substituted phenyl.

6. The method in accordance with claim 1, wherein R² is substituted heterocycloalkyl.

7. The method in accordance with claim 6, wherein heterocycloalkyl is containing one or more nitrogen as a hetero atom.

8. The method in accordance with claim 1, wherein R² is 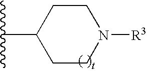
wherein
R³ is L¹-R⁴ where L¹ is C(O), S(O), S(O)₂, or a bond and R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl; and
t is an integer equal to 0, 1 or 2.

9. The method in accordance with claim 8, wherein L¹ is C(O).

10. The method in accordance with claim 8, wherein L¹ is S(O).

11. The method in accordance with claim 8, wherein L¹ is S(O)₂.

12. The method in accordance with claim 8, wherein R⁴ is C₁-C₃ alkyl, phenyl, or substituted phenyl.

13. The method in accordance with claim 1, wherein the compound is of Formula Ia: 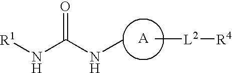
R¹ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
A is cycloalkyl, heterocycloalkyl, aryl, or heteroaryl;
L² is O, C(O), S(O), S(O)₂, or a bond; and
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
or a stereoisomer, tautomer, or pharmaceutically acceptable salt thereof.

14. The method in accordance with claim 1, wherein the compound is of Formula II: 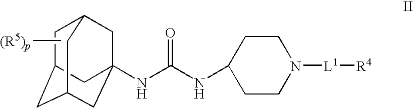
wherein:
L¹ is C(O), S(O), S(O)₂, or a bond;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl, and
R⁵ is hydrogen, halo, or hydroxy; and
p is an integer equal to 0, 1, 2 or 3;
or a stereoisomer, tautomer, or pharmaceutically acceptable salt thereof.

15. The method in accordance with claim 14, wherein L¹ is C(O).

16. The method in accordance with claim 14, wherein L¹ is S(O).

17. The method in accordance with claim 14, wherein L¹ is S(O)₂.

18. The method in accordance with claim 14, wherein R⁴ is C₁-C₃ alkyl, phenyl, substituted phenyl, or heteroaryl, and R⁵ is hydrogen or fluoro.

19. The method in accordance with claim 1, wherein the compound is of Formula III: 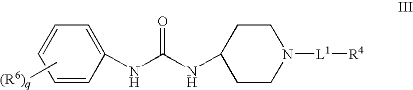
wherein:
L¹ is C(O), S(O), S(O)₂, or a bond;
q is an integer equal to 1, 2, or 3;
R⁴ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl, and
R⁶ is selected from the group consisting of halogen, haloalkyl, alkoxy, and substituted alkoxy;
or a stereoisomer, tautomer, or pharmaceutically acceptable salt thereof.

20. The method in accordance with claim 19, wherein L¹ is C(O).

21. The method in accordance with claim 19, wherein L¹ is S(O).

22. The method in accordance with claim 19, wherein L¹ is S(O)₂.

23. The method in accordance with claim 19, wherein R⁴ is C₁-C₃ alkyl, substituted C₁-C₃ alkyl, phenyl, substituted phenyl, heteroaryl, or substituted heteroaryl.

24. The method in accordance with claim 19, wherein R⁶ is halogen, CF₃, or OCF₃.

25. The method in accordance with claim 1, wherein the compound is of Formula IV: 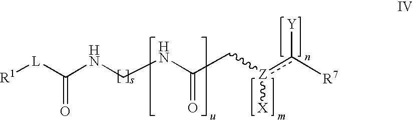
wherein
R¹ is selected from the group consisting of alkyl, substituted alkyl, cycloalkyl, substituted cycloalkyl, heterocycloalkyl, substituted heterocycloalkyl, aryl, substituted aryl, heteroaryl, and substituted heteroaryl;
L is —NH— or —CR′R″— where R′ and R″ are independently hydrogen or alkyl or R′ and R″ together form a C₃-C₆ cycloalkyl ring;
Z is C, O, or NR⁸ where R⁸ is hydrogen or C₁-C₄ alkyl and where when Z is O or NR⁸ then X is absent;
the dotted line is a single or a double bond;
the wavy line is a cis or a trans configuration when the dotted line is a double bond and m and n are 1;
when the dotted line is a single bond and Z is C, then m and n are 2;
s is 0, 1, 2, 3, 4, 5, 6, 7, 8, 9, or 10;
u is 0 or 1;
each of X and Y independently is selected from the group consisting of hydrogen, C₁-C₄ alkyl, substituted C₁-C₄ alkyl, and halo; and
R⁷ is selected from the group consisting of alkyl, substituted alkyl, acyloxy, substituted acyloxy, aminocarbonyl, carboxyl, carboxyl ester, and carboxylic acid isostere,
or a stereoisomer, tautomer, or pharmaceutically acceptable salt thereof.

26. The method in accordance with claim 25, wherein R¹ is cycloalkyl, or substituted cycloalkyl.

27. The method in accordance with claim 25, wherein R¹ is selected from the group consisting of cyclohexyl, substituted cyclohexyl, cyclooctyl, spiro[4.5]decan-8-yl, and 4-methylbicyclo[2.2.2]octan-1-yl.

28. The method in accordance with claim 25, wherein R¹ is adamantyl or substituted adamantyl.

29. The method in accordance with claim 25, wherein R¹ is phenyl or substituted phenyl.

30. The method in accordance with claim 25, wherein L is —NH—.

31. The method in accordance with claim 24, wherein L is —CR′R″— where R′ and R″ are independently H or alkyl or R′ and R″ together form a C₃-C₆ cycloalkyl ring.

32. The method in accordance with claim 25, wherein s is 2, 4, 5, 6, 7, or 8.

33. The method in accordance with claim 25, wherein s is 4.

34. The method in accordance with claim 25, wherein u is 0.

35. The method in accordance with claim 25, wherein u is 1.

36. The method in accordance with claim 25, wherein the dotted line [symbol] is the single bond, when Z is C, and u is 0, then at least one of Y is halo or C₁-C₄ alkyl.

37. The method in accordance with claim 25, wherein when Z is C and u is 1, then each of X and Y independently is hydrogen or C₁-C₄ alkyl.

38. The method in accordance with claim 25, wherein when Z is C and u is 0, then at least one of Y is methyl.

39. The method in accordance with claim 25, wherein when Z is C and u is 0, then at least one of Y is fluoro.

40. The method in accordance with claim 25, wherein R⁷ is substituted alkyl.

41. The method in accordance with claim 40, wherein substituted alkyl is —CH₂OR⁹ where R⁹ is hydrogen or C₁-C₄ alkyl.

42. The method in accordance with claim 25, wherein R⁷ is —COOR¹⁰ where R¹⁰ is hydrogen, or C₁-C₄ alkyl.

43. The method in accordance with claim 25, wherein R⁷ is —CONH₂.

44. The method in accordance with claim 25, wherein the compound is of Formula V: 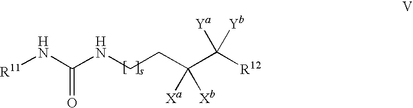
wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —OR¹³, —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
each of X^{a}, X^{b}, Y^{a}, and Y^{b} is independently selected from the group consisting of hydrogen, C₁-C₄ alkyl, substituted C₁-C₄ alkyl, and halo;
or a stereoisomer, tautomer, or pharmaceutically acceptable salt thereof.

45. The method in accordance with claim 44, wherein at least one of Y^{a} and Y^{b} is halo or C₁-C₄ alkyl.

46. The method in accordance with claim 44, wherein R¹¹ is adamantyl or substituted adamantyl.

47. The method in accordance with claim 44, wherein R¹¹ is phenyl or substituted phenyl.

48. The method in accordance with claim 25, wherein the compound is of Formula VIa or VIb: 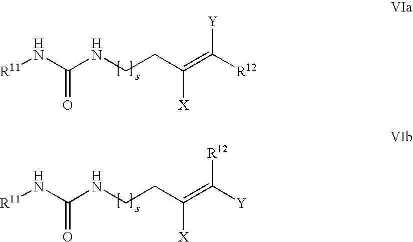
wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
X and Y are independently selected from the group consisting of hydrogen, C₁-C₄ alkyl, substituted C₁-C₄ alkyl, and halo,
or a stereoisomer, tautomer, or pharmaceutically acceptable salt thereof.

49. The method in accordance with claim 48, wherein R¹¹ is adamantyl or substituted adamantyl.

50. The method in accordance with claim 48, wherein R¹¹ is phenyl or substituted phenyl.

51. The method in accordance with claim 25, wherein the compound is of Formula VII: 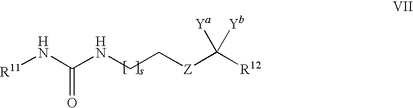
wherein
R¹¹ is selected from the group consisting of cycloalkyl, substituted cycloalkyl, phenyl and substituted phenyl;
s is 0, 1, 2, 3, 4, 5, 6, 7, 8, 9, or 10;
R¹² is selected from the group consisting of —CH₂OR¹³, —COR¹³, —COOR¹³, —CONR¹³R¹⁴, or carboxylic acid isostere; and
R¹³ and R¹⁴ are independently selected from the group consisting of hydrogen, alkyl, substituted alkyl, cycloalkyl, substituted cycloalkyl, heterocycloalkyl, substituted heterocycloalkyl, aryl, substituted aryl, heteroaryl, and substituted heteroaryl; or R¹³ and R¹⁴ together with the nitrogen atom bound thereto form a heterocycloalkyl ring having 3 to 9 ring atoms, and wherein said ring is optionally substituted with alkyl, substituted alkyl, heterocyclic, oxo or carboxy; and
Z is O or NR⁸ where R⁸ is hydrogen or C₁-C₄ alkyl; and Y^{a} and Y^{b} independently are selected from the group consisting of hydrogen, halo, and C₁-C₄ alkyl,
or a stereoisomer, tautomer, or pharmaceutically acceptable salt thereof.

52. The method in accordance with claim 51, wherein R¹¹ is adamantyl or substituted adamantyl.

53. The method in accordance with claim 51, wherein R¹¹ is phenyl or substituted phenyl.

54. The method in accordance with claim 1, wherein the compound is selected from the group consisting of
(Z)-1-(7-fluoro-8-hydroxyoct-6-enyl)-3-(adamantyl)urea;
(Z)-methyl 2-fluoro-8-(3-adamantylureido)oct-2-enoate;
(Z)-ethyl 2-fluoro-8-(3-adamantylureido)oct-2-enoate;
(Z)-isopropyl 2-fluoro-8-(3-adamantylureido)oct-2-enoate;
(Z)-2-fluoro-8-(3-adamantylureido)oct-2-enoic acid;
(Z)-2-fluoro-8-(3-adamantylureido)oct-2-enamide;
(Z)-1-(7-fluoro-8-methoxyoct-6-enyl)-3-adamantylurea;
(Z)-t-butyl 2-fluoro-8-(3-adamantylureido)oct-2-enoate;
(Z)-1-(7-fluoro-8-hydroxyoct-6-enyl)-3-(4-(trifluoromethyl)phenyl)urea;
(Z)-1-(7-fluoro-8-hydroxyoct-6-enyl)-3-(4-(trifluoromethoxy)phenyl)urea;
(Z)-1-(7-fluoro-8-hydroxyoct-6-enyl)-3-(4-fluorophenyl)urea;
(Z)-2-fluoro-8-(3-(4-fluorophenyl)ureido)oct-2-enamide;
(Z)-ethyl 2-fluoro-8-(3-(4-fluorophenyl)ureido)oct-2-enoate;
(Z)-2-fluoro-8-(3-(4-fluorophenyl)ureido)oct-2-enoic acid;
(Z)-2-fluoro-8-(3-(4-(trifluoromethoxy)phenyl)ureido)oct-2-enoic acid;
(Z)-2-fluoro-8-(3-(4-(trifluoromethoxy)phenyl)ureido)oct-2-enamide;
(Z)-ethyl 2-fluoro-8-(3-(4-(trifluoromethoxy)phenyl)ureido)oct-2-enoate;
(Z)-ethyl 2-fluoro-8-(3-(4-(trifluoromethyl)phenyl)ureido)oct-2-enoate;
2-fluoro-6-(3-adamantylureido)hexanoic acid;
Ethyl 2-fluoro-6-(3-adamantylureido)hexanoate;
1-(7-fluoro-8-hydroxyoctyl)-3-(adamantyl)urea;
1-(7,7-difluoro-8-hydroxyoctyl)-3-(adamantyl)urea;
ethyl 2,2-difluoro-8-(3-adamantylureido)octanoate;
methyl 2-fluoro-8-(3-adamantylureido)octanoate;
ethyl 2-fluoro-8-(3-adamantylureido)octanoate;
isopropyl 2-fluoro-8-(3-adamantylureido)octanoate;
2-fluoro-8-(3-adamantylureido)octanoic acid;
t-butyl 2-fluoro-8-(3-adamantylureido)octanoate;
2-fluoro-8-(3-adamantylureido)octanamide;
1-(7-fluoro-8-methoxyoctane)-3-adamantylurea;
1-(7-fluoro-8-oxononyl)-3-adamantylurea;
2-fluoro-12-(3-adamantylureido)dodecanoic acid;
Ethyl 2-fluoro-12-(3-adamantylureido)dodecanoate;
2-fluoro-10-(3-adamantylureido)decanoic acid;
Ethyl 2-fluoro-10-(3-adamantylureido)decanoate;
ethyl 2-fluoro-8-(3-(4-fluorophenyl)ureido)octanoate;
2-fluoro-8-(3-(4-fluorophenyl)ureido)octanoic acid;
2-fluoro-8-(3-(4-fluorophenyl)ureido)octanamide;
2-fluoro-8-(3-(4-(trifluoromethoxy)phenyl)ureido)octanoic acid;
ethyl 2-fluoro-8-(3-(4-(trifluoromethoxy)phenyl)ureido)octanoate;
ethyl 2-fluoro-8-(3-(4-(trifluoromethyl)phenyl)ureido)octanoate;
ethyl 2-fluoro-8-(3-(4-fluorophenyl)ureido)octanoate;
8-(3-(4,4-dimethylcyclohexyl)ureido)-2-fluorooctanoic acid;
ethyl 8-(3-(4,4-dimethylcyclohexyl)ureido)-2-fluorooctanoate;
8-(3-cyclooctylureido)-2-fluorooctanoic acid;
ethyl 8-(3-cyclooctylureido)-2-fluorooctanoate;
8-(3-(4,4-difluorocyclohexyl)ureido)-2-fluorooctanoic acid;
ethyl 8-(3-(4,4-difluorocyclohexyl)ureido)-2-fluorooctanoate;
2-fluoro-8-(3-spiro[4.5]decan-8-ylureido)octanoic acid;
ethyl 2-fluoro-8-(3-spiro[4.5]decan-8-ylureido)octanoate;
2-fluoro-8-(3-(4-methylbicyclo[2.2.2]octan-1-yl)ureido)octanoic acid;
ethyl 2-fluoro-8-(3-(4-methylbicyclo[2.2.2]octan-1-yl)ureido)octanoate;
methyl 2,2-dimethyl-11-(3-(4-(trifluoromethyl)phenyl)ureido)undecanoate;
2,2-dimethyl-11-(3-(4-(trifluoromethyl)phenyl)ureido)undecanoic acid;
1-(8-hydroxy-8-methylnonyl)-3-adamantylurea;
1-(8-hydroxy-9,9-dimethyldecyl)-3-adamantylurea;
(E)-ethyl 8-(3-adamantylureido)oct-2-enoate;
ethyl 2-methyl-8-(3-adamantylureido)octanoate;
1-(5-(2-hydroxyethoxy)pentyl)-3-adamantylurea;
methyl 2-(methyl(9-(3-(4-(trifluoromethyl)phenyl)ureido)nonyl)amino)acetate;
methyl 2-(methyl(9-(3-adamantylureido)nonyl)amino)acetate;
2-(methyl(9-(3-(4-(trifluoromethyl)phenyl)ureido)nonyl)amino)acetamide;
2-(methyl(9-(3-adamantylureido)nonyl)amino)acetamide;
ethyl 2,2-difluoro-2-(5-(3-adamantylureido)pentyloxy)acetate;
3,3-dimethyl-5-oxo-5-(6-(3-(4-(trifluoromethyl)phenyl)ureido)hexylamino)pentanoic acid;
3,3-dimethyl-5-oxo-5-(6-(3-adamantylureido)hexylamino)pentanoic acid;
ethyl 8-(3-adamantylureido)octanoate; and
1-(8-methoxyoctyl)-3-adamantylurea,
or a stereoisomer, a tautomer, or a pharmaceutically acceptable salt thereof.

55. The method in accordance with claim 1, wherein the compound is selected from the group consisting of
1-(3,4-Difluoro-phenyl)-3-[1-(4-morpholin-4-yl-butyryl)-piperidin-4-yl]-urea;
1-(1-Acetyl-piperidin-4-yl)-3-(4-trifluoromethyl-phenyl)-urea;
1-(1-Methanesulfonyl-piperidin-4-yl)-3-(4-trifluoromethyl-phenyl)-urea;
1-[1-(3-Methyl-butyryl)-piperidin-4-yl]-3-(4-trifluoromethyl-phenyl)-urea;
1-(4-Fluoro-phenyl)-3-[1-(pyridine-3-carbonyl)-piperidin-4-yl]-urea;
1-[1-(Pyridine-3-carbonyl)-piperidin-4-yl]-3-(4-trifluoromethyl-phenyl)-urea;
1-[1-(Pyridine-2-carbonyl)-piperidin-4-yl]-3-(4-trifluoromethyl-phenyl)-urea;
4-{4-[3-(4-Fluoro-phenyl)-ureido]-piperidine-1-carbonyl}-benzoic acid;
4-{4-[3-(4-Trifluoromethyl-phenyl)-ureido]-piperidine-1-carbonyl}-benzoic acid;
1-(4-Fluoro-phenyl)-3-[1-(3-trifluoromethyl-benzenesulfonyl)-piperidin-4-yl]-urea;
1-(1-Benzenesulfonyl-piperidin-4-yl)-3-(4-fluoro-phenyl)-urea;
1-(4-Fluoro-phenyl)-3-[1-(4-trifluoromethyl-benzenesulfonyl)-piperidin-4-yl]-urea;
4-{4-[3-(4-Chloro-phenyl)-ureido]-piperidine-1-sulfonyl}-benzoic acid;
4-{4-[3-(4-Trifluoromethyl-phenyl)-ureido]-piperidine-1-sulfonyl}-benzoic acid;
1-(1-Benzenesulfonyl-piperidin-4-yl)-3-(4-trifluoromethyl-phenyl)-urea;
1-[1-(4-Chloro-benzenesulfonyl)-piperidin-4-yl]-3-(4-trifluoromethyl-phenyl)-urea;
1-[1-(4-Chloro-benzenesulfonyl)-piperidin-4-yl]-3-(4-fluoro-phenyl)-urea;
1-[1-(3-Trifluoromethyl-benzenesulfonyl)-piperidin-4-yl]-3-(4-trifluoromethyl-phenyl)-urea;
1-(1-Acetyl-piperidin-4-yl)-3-(4-fluoro-phenyl)-urea;
1-(1-Benzenesulfonyl-piperidin-4-yl)-3-(3-fluoro-phenyl)-urea;
1-[1-(4-Chloro-benzenesulfonyl)-piperidin-4-yl]-3-(3-fluoro-phenyl)-urea;
1-(1-Methanesulfonyl-piperidin-4-yl)-3-(3-trifluoromethyl-phenyl)-urea;
1-(1-Acetyl-piperidin-4-yl)-3-(3-trifluoromethyl-phenyl)-urea;
1-(1-Benzenesulfonyl-piperidin-4-yl)-3-(3-trifluoromethyl-phenyl)-urea;
1-(4-Fluoro-phenyl)-3-(1-methanesulfonyl-piperidin-4-yl)-urea;
1-(3-Fluoro-phenyl)-3-[1-(3-trifluoromethyl-benzenesulfonyl)-piperidin-4-yl]-urea;
1-[1-(4-Trifluoromethyl-benzenesulfonyl)-piperidin-4-yl]-3-(3-trifluoromethyl-phenyl)-urea;
1-[1-(4-Chloro-benzenesulfonyl)-piperidin-4-yl]-3-(3-trifluoromethyl-phenyl)-urea;
1-(3-Fluoro-phenyl)-3-(1-methanesulfonyl-piperidin-4-yl)-urea;
1-(1-Acetyl-piperidin-4-yl)-3-(3-fluoro-phenyl)-urea;
1-[1-(2-1H-Imidazol-4-yl-acetyl)-piperidin-4-yl]-3-(4-trifluoromethyl-phenyl)-urea;
1-(4-Chloro-phenyl)-3-[1-(2-1H-imidazol-4-yl-acetyl)-piperidin-4-yl]-urea;
1-[1-(1-Methyl-1H-imidazole-4-carbonyl)-piperidin-4-yl]-3-(4-trifluoromethyl-phenyl)-urea;
1-(4-Chlorophenyl)-3-(1-(4-morpholinobenzoyl)piperidin-4-yl)urea;
1-(1-(4-Morpholinobenzoyl)piperidin-4-yl)-3-(4-(trifluoromethyl)phenyl)urea;
Tert-butyl 2-methyl-2-(4-(4-(3-(4-(trifluoromethyl)phenyl)ureido)piperidine-1-carbonyl)phenoxy)propanoate;
1-(1-(2,5-Dimethyloxazole-4-carbonyl)piperidin-4-yl)-3-(4-(trifluoromethyl)phenyl)urea;
2-Methyl-2-(4-(4-(3-(4-(trifluoromethyl)phenyl)ureido)piperidine-1-carbonyl)phenoxy)propanoic acid;
1-(1-Pivaloylpiperidin-4-yl)-3-(4-(trifluoromethyl)phenyl)urea;
1-(1-(Isopropylsulfonyl)piperidin-4-yl)-3-(4-(trifluoromethyl)phenyl)urea;
1-(1-Acetyl-piperidin-3-yl)-3-adamantan-1-yl-urea;
1-Adamantan-1-yl-3-(1-methanesulfonyl-piperidin-3-yl)-urea;
1-Adamantan-1-yl-3-[1-(4-chloro-benzenesulfonyl)-piperidin-3-yl]-urea;
1-Adamantan-1-yl-3-[1-(3-trifluoromethyl-benzenesulfonyl)-piperidin-3-yl]-urea;
1-[1-(methylsulfonyl)piperidin-4-yl]-3-[4-(trifluoromethyl)phenyl]urea;
1-[1-(methylsulfonyl)piperidin-4-yl]-N′-(adamant-1-yl)urea;
1-(1-acetyl-piperidin-4-yl)-3-(1-adamantyl-methyl)-urea;
1-(1-acetylpiperidin-4-yl)-3-(cyclo-hexylmethyl)urea;
1-(1-acetylpiperidin-4-yl)-3-(4-(trifluoromethyl)benzyl)urea;
1-(1-acetylpiperidin-4-yl)-3-((tetrahydro-2H-pyran-4-yl)methyl)urea;
1-(1-acetylpiperidin-4-yl)-3-(3,4-dimethoxybenzyl)urea;
1-(1-acetylpiperidin-4-yl)-3-(8-hydroxyoctyl)urea;
1-(1-acetylpiperidin-4-yl)-3-(3,3-diphenylpropyl)urea;
methyl 4-((3-(1-acetylpiperidin-4-yl)ureido)methyl)benzoate;
1-(4-(trifluoromethyl)-phenyl)-3-(1-(5-(trifluoromethyl)-pyridin-2-yl)piperidin-4-yl)urea;
1-(4-(trifluoromethyl)-phenyl)-3-(1-(3-(trifluoromethyl)-pyridin-2-yl)piperidin-4-yl)urea;
1-(1-adamantyl)-3-(1-phenylpiperidin-4-yl)urea;
1-(1-adamantyl)-3-(1-(pyridin-4-yl)piperidin-4-yl)urea;
1-(1-phenylpiperidin-4-yl)-3-(4-(trifluoro-methyl)phenyl)urea;
2-(4-(3-(4-(trifluoro-methyl)phenyl)ureido)-piperidin-1-yl)nicotinamide;
2-(4-(3-(4-trifluoro-methylphenyl)ureido)-piperidin-1-yl)nicotinic acid;
1-(1-(thiazol-2-yl)piperidin-4-yl)-3-(4-(trifluoromethyl)phenyl)urea;
1-(1-phenylpiperidin-4-yl)-3-(4-(trifluoromethoxy)phenyl)urea;
1-(4-bromophenyl)-3-(1-phenylpiperidin-4-yl)urea;
1-(1-(4-fluorophenyl)piperidin-4-yl)-3-(4-(trifluoromethoxy)phenyl)urea;
1-adamantyl-3-(1-(2-fluorophenyl)piperidin-4-yl)urea;
1-(1-(2-fluorophenyl)piperidin-4-yl)-3-(4-(trifluoromethyl)phenyl)urea;
1-(1-acetylpiperidin-4-yl)-3-(3,5,7-trifluoroadamant-1-yl)urea;
1-(1-acetylpiperidin-4-yl)-3-(3-hydroxyadamant-1-yl)urea;
1-(1-acetylpiperidin-4-yl)-3-(3,5-difluoroadamant-1-yl)urea;
1-(1-acetylpiperidin-4-yl)-3-(3-fluoroadamant-1-yl)urea;
1-(1-acetylpiperidin-4-yl)-3-(4-hydroxyadamant-1-yl)urea;
1-(1-acetylpiperidin-4-yl)-3-(2-hydroxyadamant-1-yl)urea;
(R)-1-(1-acetylpiperidin-4-yl)-3-(4-hydroxyadamant-1-yl)urea;
(S)-1-(1-acetylpiperidin-4-yl)-3-(4-hydroxyadamant-1-yl)urea;
1-(1-acetylpiperidin-4-yl)-3-(4-oxoadamantyl)urea;
1-(1-acetylpiperidin-4-yl)-3-(4,4-difluoroadamantyl)urea;
1-(1-acetylpiperidin-4-yl)-3-(4-fluoroadamantyl)urea;
4-(4-(3-(4-(trifluoromethyl)phenyl)ureido)piperidine-1-carbonyl)benzene-sulfonamide;
4-(4-(3-(4-(trifluoromethoxy)-phenyl)ureido)-piperidine-1-carbonyl)benzenesulfonamide;
4-(4-(3-(1-adamantyl)ureido)-piperidine-1-carbonyl)benzene-sulfonamide;
3-(4-(3-(1-adamantyl)ureido)-piperidine-1-carbonyl)benzene-sulfonamide;
3-(4-(3-(1-adamantyl)ureido)-piperidine-1-carbonyl)-N-methylbenzene-sulfonamide;
3-(4-(3-(4-(trifluoromethyl)phenyl)ureido)piperidine-1-carbonyl)benzene-sulfonamide;
4-(4-(3-(4-(trifluoromethyl)phenyl)ureido)piperidine-1-carbonyl)-N-methylbenzene-sulfonamide;
4-(4-(3-(1-adamantyl)ureido)-piperidine-1-carbonyl)-N-methylbenzene-sulfonamide;
N-methyl-3-(4-(3-(4-(trifluoromethyl)phenyl)ureido)piperidine-1-carbonyl)benzene-sulfonamide;
2-(4-chlorophenyl)-N-(1-(3-(N-methyl-sulfamoyl)benzoyl)-piperidin-4-yl)acetamide;
N-methyl-3-(4-(3-(4-(trifluoromethoxy)-phenyl)ureido)-piperidine-1-carbonyl)benzene-sulfonamide;
4-(4-(3-(4-fluorophenyl)ureido)piperidine-1-carbonyl)-N-methylbenzene-sulfonamide;
tert-butyl 4-(3-(4-(morpholinosulfonyl)-phenyl)ureido)-piperidine-1-carboxylate;
1-(1-acetylpiperidin-4-yl)-3-(4-(morpholinosulfonyl)phenyl)urea;
tert-butyl 4-(3-quinolin-6-yl-ureido)piperidine-1-carboxylate;
tert-butyl 4-(3-1H-indol-6-yl-ureido)piperidine-1-carboxylate;
tert-butyl 4-(3-pyridin-4-yl-ureido)piperidine-1-carboxylate;
1-(1-acetylpiperidin-4-yl)-3-(quinolin-6-yl)urea;
tert-butyl 4-(3-(2,3-dihydro-1H-inden-5-yl)ureido)-piperidine-1-carboxylate;
1-(1-acetyl-piperidin-4-yl)-3-(2,3-dihydro-1H-inden-5-yl)urea;
1-(1-acetyl-piperidin-4-yl)-3-(pyridin-4-yl)urea;
tert-butyl 4-(3-(4-(1H-tetrazol-5-yl)phenyl)-ureido)piperidine-1-carboxylate;
1-(4-(1H-tetrazol-5-yl)phenyl)-3-(1-acetylpiperidin-4-yl)urea;
1-(1-acetylpiperidin-4-yl)-3-(pyridin-2-yl)urea;
1-(1-acetylpiperidin-4-yl)-3-(6-methoxypyridin-3-yl)urea;
1-(1-acetylpiperidin-4-yl)-3-(pyridin-3-yl)urea;
1-(6-methoxypyridin-3-yl)-3-(1-pivaloylpiperidin-4-yl)urea;
tert-butyl 4-(3-(2-methylbenzo[d]thiazol-6-yl)ureido)piperidine-1-carboxylate;
1-(1-acetylpiperidin-4-yl)-3-(2-methylbenzo[d]thiazol-6-yl)urea;
methyl 5-(3-(1-acetylpiperidin-4-yl)ureido)thiophene-2-carboxylate;
tert-butyl 4-(3-(5-(methoxycarbonyl)thiophen-2-yl)ureido)piperidine-1-carboxylate;
tert-butyl 4-(3-(5-(methoxycarbonyl)furan-2-yl)ureido)piperidine-1-carboxylate;
1-(1-acetylpiperidin-4-yl)-3-(2,3-dihydrobenzo[b][1,4]dioxin-6-yl)urea;
1-(1-adamantyl)-3-(1-(4-methoxyphenylsulfonyl)-piperidin-4-yl)urea;
1-(1-picolinoylpiperidin-4-yl)-3-(4-(trifluoro-methoxy)phenyl)urea;
1-(1-acetylpiperidin-4-yl)-3-(4-tert-butyl-cyclohexyl)urea;
1-(1-acetylpiperidin-4-yl)-3-(4-ethylcyclohexyl)urea;
1-(1-acetylpiperidin-4-yl)-3-(decahydronaphthalen-2-yl)urea;
1-(1-acetylpiperidin-4-yl)-3-(4,4-dimethyl-cyclohexyl)urea;
1-(1-acetylpiperidin-4-yl)-3-(bicyclo[2.2.1]heptan-2-yl)urea;
1-(1-adamantyl)-3-(1-(2,5-dimethyloxazole-4-carbonyl)piperidin-4-yl)urea;
tert-butyl 4-(3-(4-phenoxyphenyl)ureido)-piperidine-1-carboxylate;
tert-butyl 4-(3-(4-propoxyphenyl)ureido)-piperidine-1-carboxylate;
1-(1-acetylpiperidin-4-yl)-3-(4-propoxyphenyl)urea;
1-(1-acetylpiperidin-4-yl)-3-(4-phenoxyphenyl)urea;
1-(1-adamantyl)-3-(1-pivaloylpiperidin-4-yl)urea;
methyl 4-(3-(4-(trifluoromethyl)phenyl)ureido)piperidine-1-carboxylate;
ethyl 4-(3-(4-(trifluoromethyl)phenyl)ureido)piperidine-1-carboxylate;
N-(4-(trifluoromethyl)phenyl)-4-(3-(4-(trifluoro-methyl)phenyl)ureido)-piperidine-1-carboxamide;
tert-butyl 4-(3-cyclopentylureido)-piperidine-1-carboxylate;
1-(1-acetylpiperidin-4-yl)-3-cyclopentylurea;
1-(1-pivaloylpiperidin-4-yl)-3-(4-(trifluoro-methoxy)phenyl)urea;
isopropyl 4-(3-(4-(trifluoromethyl)phenyl)ureido)piperidine-1-carboxylate;
N,N-dimethyl-4-(3-(4-(trifluoromethyl)phenyl)-ureido)piperidine-1-carboxamide;
isopropyl 4-(3-(4-(trifluoromethoxy)phenyl)ureido)piperidine-1-carboxylate;
isopropyl 4-(3-(1-adamantyl)ureido)-piperidine-1-carboxylate;
1-(1-(biphenyl-4-ylsulfonyl)piperidin-4-yl)-3-adamantylurea;
1-adamantyl-3-(1-(naphthalen-262-ylsulfonyl)piperidin-4-yl)urea;
1-adamantyl-3-(1-(phenylsulfonyl)piperidin-4-yl)urea;
1-(1-(4-chlorophenylsulfonyl)piperidin-4-yl)-3-cyclohexylurea;
1-adamantyl-3-(1-(thiophen-2-ylsulfonyl)piperidin-4-yl)urea;
1-(1-(benzylsulfonyl)piperidin-4-yl)-3-adamantylurea;
1-(1-(4-tert-butylphenylsulfonyl)piperidin-4-yl)-3-adamantylurea;
1-cyclohexyl-3-(1-propionylpiperidin-4-yl)urea;
1-adamantyl-3-(1-(2-(trifluoromethyl)phenylsulfonyl)piperidin-4-yl)urea;
1-adamantyl-3-(1-(o-tolylsulfonyl)piperidin-4-yl)urea;
1-(1-(3-chloro-2-methylphenylsulfonyl)piperidin-4-yl)-3-adamantylurea;
1-(1-(2-chloro-6-methylphenylsulfonyl)piperidin-4-yl)-3-adamantylurea;
1-adamantyl-3-(1-(4-(trifluoromethyl)phenylsulfonyl)piperidin-4-yl)urea;
1-cyclohexyl-3-(1-(3,4-dichlorophenylsulfonyl)piperidin-4-yl)urea;
1-adamantyl-3-(1-(3-(trifluoromethyl)phenylsulfonyl)piperidin-4-yl)urea;
1-adamantyl-3-(1-(1-methyl-1H-imidazole-4-carbonyl)piperidin-4-yl)urea;
1-cyclohexyl-3-(1-picolinoylpiperidin-4-yl)urea;
1-adamantyl-3-(1-(4-(methylsulfonyl)phenylsulfonyl)piperidin-4-yl)urea;
1-(1-(4-chlorophenylsulfonyl)piperidin-4-yl)-3-cyclohexylurea;
1-(1-acetylpiperidin-4-yl)-3-cyclohexylurea;
1-cyclohexyl-3-(1-(3-(trifluoromethyl)phenylsulfonyl)piperidin-4-yl)urea;
4-(4-(3-adamantylureido)piperidin-1-ylsulfonyl)benzoic acid;
1-(1-(4-chlorobenzoyl)piperidin-4-yl)-3-adamantylurea;
tert-butyl 4-(3-(4-(trifluoromethyl)phenyl)ureido)piperidine-1-carboxylate;
tert-butyl 4-(3-cycloheptylureido)piperidine-1-carboxylate;
tert-butyl 4-(3-(4-(methylsulfonyl)phenyl)ureido)piperidine-1-carboxylate;
tert-butyl 4-(3-cyclobutylureido)piperidine-1-carboxylate;
tert-butyl 4-(3-(4-bromophenyl)ureido)piperidine-1-carboxylate;
1-(1-acetylpiperidin-4-yl)-3-(4-(dimethylamino)phenyl)urea;
4-(3-(1-acetylpiperidin-4-yl)ureido)benzoic acid;
4-(3-(1-(tert-butoxycarbonyl)piperidin-4-yl)ureido)benzoic acid;
1-(1-(isopropylsulfonyl)piperidin-4-yl)-3-(4-(trifluoromethoxy)phenyl)urea;
N-adamantyl-4-(3-adamantylureido)piperidine-1-carboxamide;
N-(1-acetylpiperidin-4-yl)-4-(3-adamantylureido)piperidine-1-carboxamide;
1-(1-acetylpiperidin-4-yl)-3-(4-methylbicyclo[2.2.2]octan-1-yl)urea;
1-adamantyl-3-(1-(3-hydroxypropanoyl)piperidin-4-yl)urea;
1-(1-acetylpiperidin-4-yl)-3-(4-(methylsulfonyl)phenyl)urea;
1-cyclohexyl-3-(1-(4-morpholinobutanoyl)piperidin-4-yl)urea;
1-(1-acetylpiperidin-4-yl)-3-(4,4-difluorocyclohexyl)urea;
1-(1-acetylpiperidin-4-yl)-3-cyclobutylurea;
tert-butyl 4-(3-cyclooctylureido)piperidine-1-carboxylate;
tert-butyl 4-(3-(4-(dimethylamino)phenyl)ureido)piperidine-1-carboxylate;
1,1′-(1,1′-carbonylbis(piperidine-4,1-diyl))bis(3-adamantylurea);
tert-butyl 4-(3-(4-(methoxycarbonyl)phenyl)ureido)piperidine-1-carboxylate;
tert-butyl 4-(3-(4-(pyrrolidin-1-ylmethyl)phenyl)ureido)piperidine-1-carboxylate;
methyl 4-(3-(1-acetylpiperidin-4-yl)ureido)benzoate;
1-(4-(methylsulfonyl)phenyl)-3-(1-pivaloylpiperidin-4-yl)urea;
1-(1-(4-hydroxybutanoyl)piperidin-4-yl)-3-(4-(trifluoromethoxy)phenyl)urea;
1-adamantyl-3-(1-(3,3-dimethylbutanoyl)piperidin-4-yl)urea;
1-adamantyl-3-(1-(4-hydroxybutanoyl)piperidin-4-yl)urea;
1-adamantyl-3-(1-(3-hydroxypropylsulfonyl)piperidin-4-yl)urea;
1-(1-(3-hydroxypropylsulfonyl)piperidin-4-yl)-3-(4-(trifluoromethoxy)phenyl)urea;
1-adamantyl-3-(1-(2-methoxyacetyl)piperidin-4-yl)urea;
1-(1-(tert-butylsulfonyl)piperidin-4-yl)-3-(4-(trifluoromethoxy)phenyl)urea;
1-(1-(tert-butylsulfonyl)piperidin-4-yl)-3-adamantylurea;
1-(1-(morpholine-4-carbonyl)piperidin-4-yl)-3-(4-(trifluoromethoxy)phenyl)urea;
1-(1-acetylpiperidin-4-yl)-3-(4-cyanophenyl)urea;
1-(4-cyanophenyl)-3-(1-pivaloylpiperidin-4-yl)urea;
1-adamantyl-3-(1-(morpholine-4-carbonyl)piperidin-4-yl)urea;
1-(1-acetylpiperidin-4-yl)-3-(spiro[4.5]decan-8-yl)urea;
1-(1-acetylpiperidin-4-yl)-3-cyclooctylurea;
2-(4-chlorophenyl)-N-(1-(3-(N-methyl-sulfamoyl)benzoyl)-piperidin-4-yl)acetamide;
tert-butyl 4-(3-(4-morpholinophenyl)ureido)piperidine-1-carboxylate;
1-(1-acetylpiperidin-4-yl)-3-(4-morpholinophenyl)urea;
1-(1-acetylpiperidin-4-yl)-3-(adamantyl)urea;
1-(1-(pyridin-3-ylsulfonyl)piperidin-4-yl)-3-(4-(trifluoromethoxy)phenyl)urea;
1-(1-nicotinoylpiperidin-4-yl)-3-(4-(trifluoromethoxy)phenyl)urea;
1-(adamantyl)-3-(1-picolinoylpiperidin-4-yl)urea;
1-adamantyl-3-(5-(2-(2-ethoxyethoxy)ethoxy)pentyl)urea;
1-adamantyl-3-(8-hydroxyoctyl)urea;
1-(1-(3,3-dimethylbutanoyl)piperidin-4-yl)-3-(4-(trifluoromethyl)phenyl)urea;
1-(6-phenoxypyridin-3-yl)-3-(4-(trifluoromethyl)phenyl)urea;
1-(4-(phenylsulfonyl)phenyl)-3-(4-(trifluoromethyl)phenyl)urea;
4-(4-(3-(adamantyl)ureido)phenoxy)benzoic acid;
4-(4-(3-(adamantyl)ureido)cyclohexyloxy)benzoic acid; and
1-(3-(morpholine-4-carbonyl)phenyl)-3-(4-(trifluoromethyl)phenyl)urea; or a stereoisomer, a tautomer, or a pharmaceutically acceptable salt thereof.

56. The method in accordance with claim 1, wherein the compound of Formula I is a soluble epoxide hydrolase inhibitor having an IC₅₀ value of less than 25 μM.

57. The method in accordance with claim 1, wherein the compound of Formula I has an IC₅₀ value of less than 10 μM.

58. The method in accordance with claim 1, wherein the compound of Formula I has an IC₅₀ value of less than 1 μM.

59. The method in accordance with claim 1, wherein said disease related to endothelial dysfunction is selected from the group consisting of vascular inflammation, atherosclerosis plaque progression/rupture, acute coronary syndrome, coronary-angina, cerebral-subarachnoid hemorrhage, nephropathy, diabetic vasculopathy, and autoimmune vasculitis.

60. The method in accordance with claim 59, wherein said autoimmune vasculitis relates to scleroderma, lupus, behcet syndrome, takayashu arteritis, churg-strauss syndrome, cutaneous vasculitis, thrombangitis obliterans, sickle cell anemia and beta thalasemia.

61. The method in accordance with claim 59, wherein said disease related to endothelial dysfunction is vascular inflammation.

62. A method for chronic augmentation of endothelial nitric oxide (NO) in a patient exhibiting insufficient endothelial NO levels, which method comprises:
a) identifying a patient with insufficient endothelial NO levels;
b) administering to said patient an amount of an sEH inhibitor effective to provide a sustained increase in endothelial NO levels in said patient.
